(19) United States
(12) Patent Application Publication
(10) Pub. No.: US 2016/0361365 A1
(43) Pub. Date: Dec. 15, 2016

(54) TREATMENT OF CNS DISEASE WITH ENCAPSULATED INDUCIBLE CHOROID PLEXUS CELLS

(71) Applicant: Living Cell Technologies New Zealand Limited, Papatoetoe (NZ)

(72) Inventors: Jacqueline Eunyoung Lee, Boulder, CO (US); Kenneth Martin Taylor, Auckland (NZ); Rupa Hemant Walanj, Auckland (NZ); Bowl Bowl Janice Lam, Papakura (NZ)

(21) Appl. No.: 15/154,709

(22) Filed: May 13, 2016

Related U.S. Application Data
(60) Provisional application No. 62/162,390, filed on May 15, 2015.

(51) Int. Cl. A61K 35/30; A61K 9/00; A61K 45/06; A61K 9/48

(52) U.S. Cl. CPC A61K 35/30; A61K 9/48; A61K 9/0085; A61K 45/06

(57) ABSTRACT

Compositions and methods are disclosed that relate to improved treatments for nervous system diseases and disorders using CNS-implanted semi-permeable biocompatible capsules containing encapsulated pathogen-free xenogeneic choroid plexus (CP) cells that are induced to produce altered (and in certain embodiments increased) levels of one or more cerebrospinal fluid (CSF) components. Capsules are selected as disclosed to be capable of induction of elevated CSF production levels by CP cells that are remarkably (>16 months post implant) long-lived, without eliciting immunological rejection, inflammation or foreign body response reactions.

### BACKGROUND

### Technical Field

The present disclosure relates generally to treatment of neurological diseases and disorders including neurodegenerative diseases. More specifically, compositions and methods are described pertaining to central nervous system (CNS) implants comprising semi-permeable capsules containing surprisingly long-lived xenogeneic choroid plexus (CP) cells that can unexpectedly be induced to produce altered (e.g., increased or decreased in a statistically significant manner) levels of cerebrospinal fluid (CSF) components, which for certain preferred embodiments will be increased levels of particular CSF components. The capsules are non-immunogenic, to minimize local inflammatory reactions and avoid the need for adjuvant immunosuppressive therapy.

### Description of the Related Art

Diseases and disorders of the nervous system represent significant medical, social and economic challenges for which effective remedies remain elusive. Neurodegenerative diseases are often associated with aging and may be characterized by the progressive loss of neuronal cells from the central nervous system (CNS) and/or the peripheral nervous system (PNS), often accompanied by depression or dementia and deterioration or loss of one or more of memory, motor skills, cognitive skills, and sensory abilities, along with other neurological deficits (Suksuphew et al., 2015 World J. Stem Cells 7:502; Schadt et al., 2014 Front. Pharmacol. 5:252). Alzheimer's disease, Parkinson's disease, Huntington's disease, schizophrenia, and other nervous system diseases have become societal burdens of growing prevalence and increasing impact on healthcare costs.

Disease-related degeneration of nervous system cells, which in healthy individuals are important contributors to normal nervous system maintenance and activity, can lead to compromised nervous system functions with deleterious consequences. For example, damage to or loss of nervous system cells that secrete significant bioactive molecules such as growth factors, differentiation factors, tissue repair factors, neurotransmitters, detoxifying proteins, protein chaperones or the like, can result in devastating diseases such as Parkinson's disease, Alzheimer's disease, amyotrophic lateral sclerosis (ALS) and other conditions. When the normal functions of the lost cells involve homeostatic maintenance of a physiological state or appropriate responses to changing physiological cues, therapeutic strategies that attempt simply to restore one or a small number of multiple depleted factors to patients in an unregulated manner are typically unsuccessful. Instead, an effective disease-modifying therapy should involve constantly readjusting the supply of all factors normally secreted by these cells at physiological concentrations and in a biologically responsive, regulated manner.

Recent alternative approaches for treating neurodegeneration therefore involve introducing into the CNS viable therapeutic cells that can restore, repair or functionally replace the damaged cells. By such approaches, it is believed that the replacement cells may respond flexibly and pleiotropically to environmental cues supplied by the local milieu, for instance, by progressing through quantitative and/or qualitative changes in their gene expression and protein secretion profiles as CNS cell and tissue growth, differentiation, repair and/or remodeling may proceed. Such CNS cell replacement therapies have included attempts to replace cells that have been lost due to disease directly with primary effector cells, such as fetal midbrain tissue transplants that may differentiate into dopamine-producing neurons once transplanted in patients with Parkinson's disease (Kordower et al., 1995 N Engl J Med. 332(17):1118; Lindvall, 1998 Mov Disord. 13 Suppl 1:83; Roitberg et al., 2004 Neurol Res. 26(4):355; Kefalopoulou et al., 2014 JAMA Neurol. 71(1):83; Bega et al., 2014 JAMA 311(6):617).

As a departure from direct CNS cell replacement therapy, other recently identified alternative therapies focus on supplying, to a damaged CNS tissue site, cells that are capable of ameliorating the CNS deficit indirectly. For example, choroid plexus (CP) cells have attracted particular attention in view of the recognized role of these specialized CNS cells in CSF production (e.g., Damkier et al., 2013 Physiol. Rev. 93:1847). The choroid plexus (CP) is a specialized epithelial tissue within the ventricles of the brain. One of its key functions is to secrete cerebrospinal fluid (CSF) that provides neurotrophic, neuro-protective and neuro-restorative factors, as evidenced by behavioral improvement and histological data from various small animal and non-human primate disease model studies (Redzic et al., 2005 Curr Top Dev Biol. 71:1; Borlongan et al., 2004 Stroke 35(9):2206; Emerich et al., 2006 Neurobiol Dis. 23(2):471; Borlongan et al., 2008 Cell Transplant 16(10):987; Luo et al., 2013 J Parkinsons Dis. 3(3):275).

The function and turn-over rate of CSF in the CNS decreases significantly during aging, which may contribute to many neurodegenerative diseases that occur among the elderly (Redzic et al., 2005 Curr Top Dev Biol. 71:1; Chen et al., 2009 Exp Gerontol. 44(4):289; Chen et al., 2012 Exp Gerontol. 47(4):323). Recent findings indicate that CSF circulation through the interstitial space within the brain occurs more effectively during the sleep cycle in animals (Xie et al., 2013 Science 342(6156):373), implying that reduced sleep can contribute to decreased clearance of waste byproducts and increased plaque formation, which are known to contribute to neurodegeneration (Iliff et al., 2014 J Neurosci. 34(49):16180). These findings, however, also imply that continuous production of CSF locally within a damaged site in the brain during the entire diurnal cycle may have clinically undesirable consequences. For example, excess CSF production at a damaged CNS site may result in dilution of active synaptic proteins or neurotransmitters to potentially suboptimal concentrations in the synaptic areas. It is therefore difficult to predict whether increasing CSF production would be a viable therapeutic strategy for the treatment of CNS disease.

Nevertheless, multiple efforts have been directed to the use of choroid plexus (CP) cell transplants by implantation in the CNS, by which CP-derived CSF components or other CP processes, products or metabolites may act as secondary effectors to restore damaged host tissues, most likely by reprogramming and/or restoration of various cell types in and around the implantation site. For practical and ethical reasons, CP implants have typically employed xenogeneic CP cells and therefore require the implementation of immunosuppressive, anti-inflammatory measures to counteract immunological rejection and/or host inflammation (e.g., foreign body response) reactions to the xenotransplants. Biocompatible, semi-permeable alginate capsules are known as non-immunogenic vehicles in which to introduce therapeutic cells into the brain to minimize such reactions whilst permitting soluble cell products to diffuse into the tissue surrounding the implanted capsule (e.g., U.S. Pat. No. 6,322,804, U.S. Pat. No. 5,834,001, U.S. Pat. No. 6,083,523, US2007/134224, U.S. Pat. No. 5,869,463, US2004/213768, US2009/0047325). The specific implantation in the brain of choroid plexus tissue fragments within biocompatible capsules for the treatment of CNS diseases is described, for example, in US2007/134224, and in US2004/213768 and US2009/0047325 and related patent application publications. As described, for example, in US2009/0047325, in addition to CSF production by encapsulated xenotransplanted CP cells locally at an implantation site of CNS tissue damage, the use of neonatal CP cells may provide higher concentrations of biologically active CSF molecules than would be supplied by adult CP cells, given that the CSF of newborn mammals is typically enriched in CSF components.

Despite these advances in the development of therapeutic xenotransplants, however, there remain a number of unmet challenges. For instance, xenogeneic CP tissue may be available in limited quantities, and even when neonatal CP cells are used, the quantity of elaborated CSF components following xenotransplantation may not be adequate to effect correction of the nervous system deficit.

Similarly, where extensive nervous system tissue damage is present, and/or where there is only limited space for CP-containing capsule placement in the recipient, and/or where high levels of CSF component production are desired, there is a real risk of further damaging the implantation site for encapsulated xenogeneic CP cells (e.g., a CNS site for CP-capsule implantation directly in brain tissue) if a large number of capsules must be implanted at a CNS site and/or if multiple CNS implantation sites or repeated invasive procedures would be needed to deliver a desired level of CSF production capacity.

The longevity of CP xenotransplants is also unclear from prior reports, but chronic neurodegenerative diseases may require long-term therapies. Repeated surgical interventions to replace exhausted encapsulated CP implants would be inconvenient, potentially harmful to the patient, and costly. Additionally, xenotransplantation carries the risk of undesirably introducing into the transplant recipient harmful pathogens that are present in the donor CP tissue.

These and other shortcomings of existing methodologies have hindered the development of xenotransplants for CNS therapy, in particular where prior to the present disclosure it has not been possible to predict whether implantation of encapsulated CP cells into a damaged CNS site would result in long-term beneficial clinical outcome. The presently disclosed embodiments address these needs and provide other related advantages.

### BRIEF SUMMARY

The present invention provides, in certain embodiments, a method of treating a subject known to have or suspected of having a nervous system disease, comprising (a) selecting one or more semi-permeable biocompatible capsules in which are encapsulated choroid plexus (CP) tissue fragments that are obtained by either or both of mechanical and enzymatic dissociation of mammalian choroid plexus tissue to obtain CP cell clusters that are about 50 μm to about 200 μm in diameter and that comprise CP epithelial cells, substantially all of said capsules being about 400 μm to about 800 μm in diameter and having about 200 to about 10,000 CP cells per capsule (b) administering one or a plurality of said capsules to a central nervous system (CNS) injection site or to 2, 3, 4, 5, 6, 7, 8, 9, 10, 11, or 12 CNS injection sites in the subject; and (c) prior to, simultaneously with, or subsequent to said step (b) of administering, contacting the choroid plexus tissue cells in the one or a plurality of capsules with a choroid plexus inducing agent that induces the choroid plexus tissue cells to produce one or more cerebrospinal fluid (CSF) components at a level that is altered (e.g., increased or decreased in a statistically significant manner) relative to the level at which the choroid plexus tissue cells produce said one or more cerebrospinal fluid (CSF) components without said step of contacting, and which level is in certain embodiments greater for one or more CSF components than the level at which the choroid plexus tissue cells produce said one or more cerebrospinal fluid (CSF) components without said step of contacting.

In certain other embodiments there is provided a method of treating a subject known to have or suspected of having a nervous system disease, comprising (a) selecting one or more semi-permeable biocompatible capsules in which are encapsulated in vitro differentiated choroid plexus (CP) cells that are obtained by culturing a population of pluripotent cells under conditions and for a time sufficient to obtain a plurality of in vitro differentiated choroid plexus (CP) cells, substantially all of said capsules being about 400 μm to about 800 μm in diameter and having about 200 to about 10,000 CP cells per capsule; (b) administering one or a plurality of said capsules to a central nervous system (CNS) injection site or to 2, 3, 4, 5, 6, 7, 8, 9, 10, 11, or 12 CNS injection sites in the subject; and (c) prior to, simultaneously with, or subsequent to said step (b) of administering, contacting the in vitro differentiated choroid plexus (CP) cells in the one or a plurality of capsules with a choroid plexus inducing agent that induces the in vitro differentiated choroid plexus (CP) cells to release one or more cerebrospinal fluid (CSF) components at a level that is altered (e.g., increased or decreased in a statistically significant manner) relative to the level at which the choroid plexus tissue cells produce said one or more cerebrospinal fluid (CSF) components without said step of contacting, and which level is in certain embodiments greater for one or more CSF components than the level at which the choroid plexus tissue cells produce said one or more cerebrospinal fluid (CSF) components prior to said step of contacting. In certain embodiments the step of contacting the CP cells with the choroid plexus inducing agent takes place prior to said step (b) of administering. In certain embodiments the choroid plexus inducing agent comprises one or more agents selected from (a) a Wnt signaling pathway agonist, (b) a GSK3β inhibitor, (c) a beta-catenin activator, (d) an antioxidant, and (e) 1,25-dihydroxyvitamin D₃. In certain embodiments (1) the Wnt signaling pathway agonist is selected from WAY-316606 (SFRP inhibitor), IQ1 (PP2A activator), QS11 (ARFGAP1 activator), (hetero)arylpyrimidine, or 2-amino-4-[3,4-(methylenedioxy) benzyl-amino]-6-(3-methoxyphenyl) pyrimidine, Norrin, R-spondin-1, R-spondin-2, R-spondin-3, R-spondin-4, (2) the GSK3β inhibitor is selected from SB-216763, BIO (6-bromoindirubin-3′-oxime), lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or another lithium salt that comprises cationic lithium, (3) the beta-catenin activator is selected from deoxycholic acid (DCA) and a compound of FIG. 5, and (4) the antioxidant is selected from a 10-(6′-ubiquinoyl) decyltriphenylphosphonium salt (mitoquinol, MITOQ®), ubiquinol (coenzyme Q), tocopherols, tocotrienol (vitamin E), α-tocopherol, γ-tocopherol, 2-aminoethanesulfonic acid (taurine), ascorbic acid, glutathione, and melatonin.

In certain embodiments the mammalian choroid plexus tissue is from a mammal that is xenogeneic or allogeneic relative to the subject. In certain embodiments the mammalian choroid plexus tissue comprises porcine, ovine, bovine, caprine, or non-human primate choroid plexus tissue. In certain embodiments the porcine choroid plexus tissue comprises fetal or neonatal choroid plexus tissue, which in certain further embodiments is substantially free of human pathogens. In certain related embodiments the fetal or neonatal choroid plexus tissue is substantially free of human-tropic transmissible porcine endogenous retroviruses, and in certain embodiments the fetal or neonatal choroid plexus tissue is substantially incapable of producing infectious human-tropic porcine endogenous retroviruses (PERVs), or the fetal or neonatal choroid plexus tissue is obtained from an animal that lacks PERV genes. In certain further embodiments the fetal or neonatal choroid plexus tissue is obtained from an animal that lacks a PERV-C env gene which is capable of recombination with a PERV-A env gene.

In certain embodiments of the above described methods, the population of pluripotent cells is obtained from a source that is selected from embryonic cells, umbilical cord cells, placental cells, neural crest progenitors, adult tissue stem cells, and somatic tissue cells. In certain embodiments the population of pluripotent cells is cultured in a culture medium that comprises one or more in vitro CP differentiation agents selected from a bone morphogenic protein (BMP) or a BMP signaling pathway agonist, a transforming growth factor-beta (TGF-β) superfamily member or a TGF-β signaling pathway agonist, a nodal protein or a nodal signaling pathway agonist, a mammalian growth and differentiation factor (GDF) or a GDF signaling pathway agonist, a Wnt protein ligand or a Wnt signaling pathway agonist, a fibroblast growth factor (FGF) or an FGF signaling pathway agonist, and sonic hedgehog (Shh) or a Shh signaling pathway agonist, under conditions and for a time sufficient to obtain said plurality of in vitro differentiated choroid plexus (CP) cells. In certain further embodiments the Wnt signaling pathway agonist is selected from WAY-316606 (SFRP inhibitor), IQ1 (PP2A activator), QS11 (ARFGAP1 activator), 2-amino-4-[3,4-(methylenedioxy) benzyl-amino]-6-(3-methoxyphenyl) pyrimidine, Norrin, R-spondin-1, R-spondin-2, R-spondin-3, or R-spondin-4, lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or another lithium salt that comprises cationic lithium.

In certain embodiments of the above described methods, the encapsulated in vitro differentiated choroid plexus (CP) cells are xenogeneic or allogeneic relative to the subject. In certain embodiments either or both of (i) the capsules do not elicit chronic inflammation at the CNS injection site, and (ii) administration of an immunosuppressant agent to the subject is not required to ameliorate immunological rejection of the capsules at the CNS injection site. In certain embodiments the one or more CSF components comprise at least one of (i) one or more growth factors, (ii) one or more CSF antioxidants, (iii) one or more chemotactic factors, (iv) one or more chaperone proteins, or (v) one or more CP products as presented in FIG. 7A-J. In certain further embodiments (a) the one or more growth factors are selected from growth factors that may include but need not be limited to IGF-1, IGF-II, FGF-1, bFGF (FGF-2), FGF-9, FGF-12, FGF-18, TGF-β1, TGF-β2, TGF-β3, VEGF, VEGF-2, VEGF-B, VEGF-C, EGF, growth hormone (GH), BMP-1, BMP-2, BMP-4, BMP7, BMP-11, BMP-15, GDF-1, GDF-7, GDF-8, GDF-9, nerve growth factor (NGF), PEDF (pigment epithelium derived factor, also known as SerpinF1), glucagon-like peptide-1 (GLP-1), IGF2, BDNF, NT-3, NT-4, GDF-15, GDNF, connective tissue growth factor (CTGF), axotrophin, heparin-binding EGF-like growth factor (HB-EGF), platelet derived growth factor-alpha (PDGF-α), Keratinocyte growth factor (KGF), or neurite growth-promoting factor-2/midkine (NEGF2); (b) the one or more CSF antioxidants are selected from ceruloplasmin, superoxide dismutase-1 (SOD-1), superoxide dismutase-2 (SOD-2, Mn-type), superoxide dismutase copper chaperone (CCS), DJ-1/PARK7, catalase, selenoproteins (I, M, N, P, S, T, W, X, 15 kDa), glutathione S-transferase, glutathione reductase, glutathione peroxidase, hydroxyacyl glutathione hydrolase or thioredoxin; (c) the one or more chemotactic factors are selected from chemotactic factors that may include but need not be limited to alveolar macrophage-derived chemotactic factor-I (AMCF-I), AMCF-II, stromal cell-derived factor-2, chemokine (CXC motif) ligand 2, chemokines (CCL8, CCL16, CCL19, CCL21, CCL25, CXCL2, CXCL4, CXCL9, CXCL12, CXCL13, CXCL14), chemokine (CXC motif) receptor-4, a chemokine-like factor super family (CKLF-3, -6, -7), or neurite growth-promoting factor-2/midkine (NEGF2); or (d) the chaperone proteins are selected from proteins that may include but need not be limited to transthyretin, lipocalin-type prostaglandin D synthase/β-trace (L-PGDS), apolipoproteins (A, B, C, D, E, H, J, M, N, R), lipocalin-6, lipocalin-7, cystatin B, cystatin C, cystatin EM, cystatin 11, a heat shock protein (HSP) family member, or DJ-1/PARK7.

In certain embodiments, within each capsule the CP cells are present in a core volume of less than one microliter. In certain embodiments the step of administering comprises administering one or more capsules that each contain at least about 200, 400, 600, 800, 1000, 2000, 3000, 4000, 5000, 7500 or 9000 and not more than about 10,000 CP cells. In certain further embodiments the one or more capsules each contain at least about 400, 600, 800, 1000, 1500, 2000, 2500, 3000, 3500, 4000, 4500, 5000, 5500, 6000, 6500, 7000, or 7500 and not more than about 8000 cells. In certain embodiments the step of administering comprises administering a therapeutically effective amount of the capsules to the CNS injection site, which in certain further embodiments comprises administering no more than 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 capsules to the 1, 2, 3, 4, 5, 6, 7, 8, 9, 10, 11, or 12 CNS injection sites.

In certain embodiments of the above described methods, at least 1, 5, 10, 20, 30, 40 or 50 percent of the encapsulated CP cells remain viable for at least six months after the step of administering. In certain embodiments exterior surfaces of the biocompatible capsules are substantially free of extracellular matrix deposition for at least one year after the step of administering. In certain embodiments administering the capsules to the CNS injection site comprises delivering a suspension comprising the capsules in a carrier solution, which in certain further embodiments comprises at least one of NaCl, artificial cerebrospinal fluid (CSF), ascorbate, or an anti-inflammatory agent. In certain still further embodiments the anti-inflammatory agent is selected from a non-steroidal anti-inflammatory drug (NSAID), a steroid anti-inflammatory drug, and a connexin antagonist.

In certain embodiments of the above described methods, the subject is a human or a non-human mammal. In certain embodiments the subject is known to have a nervous system disease, which in certain further embodiments is selected from (a) a neurodegenerative disease that is characterized by death of neurons, and (b) a nervous system disease that is selected from Parkinson's disease, Alzheimer's disease, Huntington's disease, amyotrophic lateral sclerosis (ALS, also known as motor neurone disease), ataxia-telangiectasia, progressive bulbar palsy, progressive muscular atrophy, dementia with Lewy bodies, multiple system atrophy, spinocerebellar ataxia type 1 (SCA 1), or an age-related neurodegenerative disorder. In certain other embodiments the nervous system disease is selected from (a) a disease that is characterized by a decrease in a level of at least one nerve cell function, relative to the level of said nerve cell function in a control subject known to be free of the nervous system disease, and (b) the disease of (a) that is selected from Parkinson's disease (dopaminergic neurons), Alzheimer's disease (noradrenergic neurons, Adori et al. 2015, Acta Neuropathol 129(4):541), Huntington's disease (medium spiny GABA neurons, MSN), amyotrophic lateral sclerosis (motor neuron disease), and depression (serotonergic neuron). In certain other embodiments the nervous system disease is selected from (a) a disease that is characterized by an increase in a level of at least one nerve cell function, relative to the level of said nerve cell function in a control subject known to be free of the nervous system disease, and (b) the disease of (a) that is selected from psychosis, schizophrenia (hyperactive dopamine signaling); epileptic seizures (glutamatergic excitotoxicity), ischemic stroke (glutamatergic excitotoxicity), and insomnia associated with restless leg syndrome (overactive glutamatergic activity). In certain other embodiments the nervous system disease is selected from (a) a disease that is characterized by presence in the subject of cerebrospinal fluid (CSF) that comprises an altered level of one or more cerebrospinal fluid (CSF) components, relative to the level of said CSF component or components in a control subject known to be free of the nervous system disease, and (b) the disease of (a) that is selected from Alzheimer's disease and diabetes mellitus. In certain other embodiments the nervous system disease is selected from (a) a disease that is characterized by presence in the subject of an altered level of at least one choroid plexus function, relative to the level of said choroid plexus function in a control subject known to be free of the nervous system disease, (b) the disease of (a) that is selected from Sturge-Weber syndrome and Klippel-Trenaunay-Weber syndrome, (c) a disease that is characterized by an increase in a level of abnormally folded protein deposits in brain tissue of the subject, relative to the level of abnormally folded protein deposits in a control subject known to be free of the nervous system disease, and (d) the disease of (c) that is selected from cerebral amyloid angiopathy, hereditary cerebral hemorrhage with amyloidosis-Icelandic type (HCHWA-I), cerebral hemorrhage with amyloidosis-Dutch type (HCHWA-D), meningocerebrovascular and oculoleptomeningeal amyloidosis, gelsolin-related spinal and cerebral amyloid angiopathy, familial amyloidosis-Finnish type (FAF), vascular variant prion cerebral amyloidosis, familial British dementia (FBD) (also known as familial cerebral amyloid angiopathy-British type or cerebrovascular amyloidosis-British type), familial Danish dementia (also known as heredopathia ophthalmo-oto-encephalica), familial transthyretin (TTR) amyloidosis, and PrP cerebral amyloid angiopathy (PrP-CAA).

In certain embodiments of the above described methods, the nervous system disease is a central nervous system (CNS) disease, which in certain embodiments is at least one of (i) a neurodegenerative disease that is characterized by death of CNS neurons, and (ii) a CNS disease characterized by a decrease in a level of at least one CNS nerve cell function, relative to the level of said CNS nerve cell function in a control subject known to be free of the CNS disease, and iii) a CNS disease characterized by an increase in a level of at least one CNS nerve cell function, relative to the level of said CNS nerve cell function in a control subject known to be free of the CNS disease, wherein said CNS neurons and CNS nerve cell are present in at least one of brain, spinal cord, retina, optic nerve, cranial nerve, olfactory nerve or olfactory epithelium.

In certain embodiments of the above described methods the nervous system disease is a peripheral nervous system (PNS) disease, which in certain embodiments is at least one of (i) a neurodegenerative disease that is characterized by death of PNS neurons, and (ii) a PNS disease characterized by a decrease in a level of at least one PNS nerve cell function, relative to the level of said PNS nerve cell function in a control subject known to be free of the PNS disease, and iii) a PNS disease characterized by an increase in a level of at least one PNS nerve cell function, relative to the level of said PNS nerve cell function in a control subject known to be free of the PNS disease, wherein said PNS neurons and PNS nerve cell are present in at least one of a peripheral ganglion or a peripheral nerve.

In certain embodiments of the above described methods the CNS injection site is in brain tissue of the subject. In certain embodiments the CNS injection site is in a brain ventricle of the subject. In certain embodiments the CNS injection site in the subject is selected from (a) a CNS site and preferably a CNS injection site that comprises a target site for nerve cell fibers that are affected by the nervous system disease, (b) a CNS site and preferably a CNS injection site that contains neuronal cells that are at risk of dying due to the nervous system disease, (c) a CNS site and preferably a CNS injection site that contains neuronal cells that are at risk of a decrease in a level of at least one nerve cell function, relative to the level of said nerve cell function in a control subject known to be free of the nervous system disease, (d) a CNS site and preferably a CNS injection site that contains neuronal cells that are at risk of an increase in a level of at least one nerve cell function, relative to the level of said nerve cell function in a control subject known to be free of the nervous system disease, (e) a CNS site and preferably a CNS injection site that is selected so that the capsules are substantially free of contact with blood, and (f) a CNS site and preferably a CNS injection site that is selected so that CSF components secreted by the capsules subsequent to the step of administering are distributed by CSF circulation throughout the subject's brain.

In certain embodiments of the above described methods administration takes place at a PNS site such as a PNS injection site. In certain embodiments the PNS injection site in the subject is selected from (a) a PNS site and preferably a PNS injection site that comprises a target site for nerve cell fibers that are affected by the nervous system disease, (b) a PNS site and preferably a PNS injection site that contains neuronal cells that are at risk of dying due to the nervous system disease, (c) a PNS site and preferably a PNS injection site that contains neuronal cells that are at risk of a decrease in a level of at least one nerve cell function, relative to the level of said nerve cell function in a control subject known to be free of the nervous system disease, (d) a PNS site and preferably a PNS injection site that contains neuronal cells that are at risk of an increase in a level of at least one nerve cell function, relative to the level of said nerve cell function in a control subject known to be free of the nervous system disease, (e) a PNS site and preferably a PNS injection site that is selected so that the capsules are substantially free of contact with blood, and (f) a PNS site and preferably a PNS injection site that is selected so that CSF components secreted by the capsules subsequent to the step of administering are distributed in or around the PNS tissue in the vicinity of the PNS injection site.

In certain embodiments of the above described methods, the biocompatible capsule comprises a core layer of a high mannuronic acid alginate cross-linked with a cationic cross-linking agent, an intermediate layer of polycations forming a semi-permeable membrane, and an outer layer of a high mannuronic acid alginate cross-linked with a cationic cross-linking agent, wherein the high mannuronic acid alginate in the core and outer layers is the same or different and contains between from about 50% to about 95% mannuronic acid residues, wherein the polycation layer is not comprised of poly-L-lysine. In certain further embodiments the high mannuronic acid alginate has an average molecular weight of greater than about 300 kDa and not more than 1000 kDa and the polycation layer is formed from a polycationic agent having an average molecular weight of between 10 and 40 kDa.

In certain embodiments of the above described methods, administering the capsules to the CNS injection site (or in certain other embodiments, to a PNS injection site) comprises delivering the capsules through a catheter. In certain further embodiments, delivering comprises controllably positioning the catheter with a stereotactic apparatus. In certain still further embodiments, the stereotactic apparatus may comprise a stereotactic apparatus or a modified stereotactic apparatus by way of exemplary illustration and not limitation, a deep brain stimulator (DBS) microdriver, a “frameless” stereotactic head frame, a skull-mounted aiming device, a Leksell frame, a Cosman-Roberts-Wells frame, or another similar modified stereotactic apparatus or the like or any equivalent. In certain embodiments the catheter comprises an external catheter, an obdurator, a plunger, and a delivery catheter.

In certain embodiments of the above described methods the nervous system disease is selected from Parkinson's disease, Alzheimer's disease, Huntington's disease, amyotrophic lateral sclerosis (ALS), prion disease, motor neuron disease, spinocerebellar ataxia, spinal muscular atrophy, multiple system atrophy-Parkinson type, multiple system atrophy-cerebellar type, essential tremor, progressive supronuclear palsy, dyskinesias, dementia with Lewy bodies, essential tremor, drug-induced Parkinsonism, ataxia-telangiectasia, spinocerebellar ataxia, cerebellar degeneration, cerebral atrophy, olivopotocerebellar atrophy, corticobasal degeneration, dyssynergia cerebellaris myoclonica, Friedreich's ataxia; a static nervous diseases, stroke, central pain syndrome, chronic pain, migraine, glossopharyngeal neuralgia, a seizure disorder, epilepsy, cerebral palsy; a trauma-related CNS diseases, Gerstmann's syndrome, locked-in syndrome, spinal cord injury, a progressive neurodegenerative diseases, progressive neurodegenerative disease associated with aging and dementia, Alzheimers disease, Parkinson's disease, frontotemporal dementia, Gerstmann-Straussler-Scheinker disease, giant axonal neuropathy, hereditary neuropathies, infantile neuroaxonal dystrophy, Krabbe disease, Landau-Kleffner syndrome, Tabes dorsalis, a disease of motor neurons and neuromuscular junctions, spinal muscular atrophy, Kennedy's disease, monomelic amyotrophy, dystonias, hereditary spastic paraplegia, Isaacs' syndrome, Lambert-Eaton myasthenic syndrome, motoneuron diseases, restless legs syndrome, Tourette syndrome; inflammatory diseases of the CNS, multiple sclerosis; drug or toxin-induced CNS diseases, neuroleptic malignant syndrome, tardive dyskinesia, Wilson disease, neurotoxicity; nervous system disease of metabolic failure, Refsum disease, a nervous system infectious disease, meningitis, acute disseminated encephalomyelitis, Guillain-Barre syndrome, neurological complications of AIDS, botulism, tetanus, neurosyphilis, poliomyelitis, rabies, HIV/AIDS, prion diseases, Naegleria fowleri (amoebic brain infection); neurocysticerosis; a neuropsychiatric disease, depression, mood disorders; obsessive-compulsive disorder, eating disorder, addiction, anxiety-related disorder, bipolar disorder, attention-deficit-hyperactivity disorder, autism, schizophrenia; a neuroendocrine disease, narcolepsy, insomnia, a disease associated with or characterized by one or more of neuronal death, glutamate toxicity, protein aggregates or deposits, or amyloid plaque formation, cerebral amyloid angiopathy, hereditary cerebral hemorrhage with amyloidosis-Icelandic type (HCHWA-I), cerebral hemorrhage with amyloidosis-Dutch type (HCHWA-D), meningocerebrovascular and oculoleptomeningeal amyloidosis, gelsolin-related spinal and cerebral amyloid angiopathy, familial amyloidosis-Finnish type (FAF), vascular variant prion cerebral amyloidosis, familial British dementia (FBD) (also known as familial cerebral amyloid angiopathy-British type or cerebrovascular amyloidosis-British type), familial Danish dementia (also known as heredopathia ophthalmo-oto-encephalica), familial transthyretin (TTR) amyloidosis, PrP cerebral amyloid angiopathy (PrP-CAA); a nervous system disease of mitochondrial dysfunction, a nervous system disease of mitochondrial dysfunction that comprises reactive oxygen species (ROS) production levels in excess of ROS production levels found in normal, healthy control subjects; a brain derived neurotrophic factor-related disorders, bipolar disorders, Rett Syndrome, and Rubinstein-Taybi Syndrome.

Turning to another embodiment there is provided a method of treating a subject known to have or suspected of having a nervous system disease, comprising: (a) selecting one or more semi-permeable biocompatible capsules in which are encapsulated choroid plexus (CP) tissue fragments that are obtained by either or both of mechanical and enzymatic dissociation of mammalian choroid plexus tissue to obtain CP cell clusters that are about 50 μm to about 200 μm in diameter and that comprise CP epithelial cells, substantially all of said capsules being about 400 μm to about 800 μm in diameter and having about 200 to about 10,000 CP cells per capsule; (b) administering one or a plurality of said capsules to a peripheral nervous system (PNS) injection site or to 2, 3, 4, 5, 6, 7, 8, 9, 10, 11, or 12 PNS injection sites in the subject; and (c) prior to, simultaneously with, or subsequent to said step (b) of administering, contacting the choroid plexus tissue cells in the one or a plurality of capsules with a choroid plexus inducing agent that induces the choroid plexus tissue cells to produce one or more cerebrospinal fluid (CSF) components at a level that is altered relative to the level at which the choroid plexus tissue cells produce said one or more cerebrospinal fluid (CSF) components without said step of contacting.

In certain other embodiments there is provided a method of treating a subject known to have or suspected of having a nervous system disease, comprising: (a) selecting one or more semi-permeable biocompatible capsules in which are encapsulated in vitro differentiated choroid plexus (CP) cells that are obtained by culturing a population of pluripotent cells under conditions and for a time sufficient to obtain a plurality of in vitro differentiated choroid plexus (CP) cells, substantially all of said capsules being about 400 μm to about 800 μm in diameter and having about 200 to about 10,000 CP cells per capsule; (b) administering one or a plurality of said capsules to a peripheral nervous system (PNS) injection site or to 2, 3, 4, 5, 6, 7, 8, 9, 10, 11, or 12 PNS injection sites in the subject; and (c) prior to, simultaneously with, or subsequent to said step (b) of administering, contacting the in vitro differentiated choroid plexus (CP) cells in the one or a plurality of capsules with a choroid plexus inducing agent that induces the in vitro differentiated choroid plexus (CP) cells to release one or more cerebrospinal fluid (CSF) components at a level that is altered relative to the level at which the choroid plexus tissue cells produce said one or more cerebrospinal fluid (CSF) components prior to said step of contacting.

In certain further embodiments, the choroid plexus inducing agent induces production of one or more CSF components at a level that is greater than the level at which the choroid plexus tissue cells produce said one or more cerebrospinal fluid (CSF) components without said step of contacting.

These and other aspects of the herein described invention embodiments will be evident upon reference to the following detailed description and attached drawings. All of the U.S. patents, U.S. patent application publications, U.S. patent applications, foreign (non-U.S.) patents, foreign patent applications and non-patent publications referred to in this specification and/or listed in the Application Data Sheet are incorporated herein by reference in their entirety as if each was incorporated individually. Aspects and embodiments of the invention can be modified, if necessary, to employ concepts of the various patents, applications and publications to provide yet further embodiments.

### BRIEF DESCRIPTION OF THE SEVERAL VIEWS OF THE DRAWINGS

FIG. 1 shows VEGF secretion (pg VEGF/μg DNA) into culture medium in vitro by random samples (non-selected) of capsules comprising alginate-encapsulated porcine choroid plexus (CP) cells, compared to capsules comprising alginate-encapsulated porcine choroid plexus (CP) cells that were selected as described herein.

FIG. 2 shows total antioxidant capacity (TAC) of porcine choroid plexus (CP) cell clusters stimulated by exposure for 72 hours to the indicated candidate CP inducing agents.

FIG. 3 shows (FIG. 3A) total antioxidant capacity (TAC) of porcine choroid plexus (CP) cell clusters stimulated by exposure for 72 hours to the indicated concentration of lithium chloride (LiCl), normalized to media control values. FIG. 3B shows total antioxidant capacity (TAC) of encapsulated porcine choroid plexus (CP) cell clusters stimulated by exposure for 72 hours to the indicated concentration of lithium chloride (LiCl), lithium carbonate, taurine and mitoquinol (MitoQ®).

FIG. 4 shows immunoperoxidase staining of PEDF (pigment epithelium derived factor, small arrows) in representative histological cross sections of rat brains implanted with selected alginate encapsulated CP cells for 12 (FIG. 4A) or 16 (FIG. 4B) months, indicating that CP cells within the capsules survived in vivo for 16 months while maintaining CP functional characteristics. Capsule wall (large arrow) shows no evidence of fibrotic scarring or cellular immune response.

FIG. 5(A-KK) shows exemplary beta-catenin activators that are disclosed in U.S. Application Publication No. US/2014/0187510.

FIG. 6(A-C) shows exemplary in vitro CP differentiation agents.

FIG. 7(A-J) shows exemplary CP products that may occur as CSF components.

FIG. 8(A-K) shows exemplary CSF component-encoding genes the expression of which is altered (e.g., increased (8A-E) or decreased (8F-K) in a statistically significant manner relative to controls) in choroid plexus (CP) cell clusters stimulated by exposure for 72 hours with an inducing agent (LiCl).

### DETAILED DESCRIPTION

The present invention is directed in certain embodiments as described herein to compositions and methods for treating nervous system diseases or disorders, including neurodegenerative and other neurological diseases.

These and related embodiments are based on the unexpected findings that mammalian choroid plexus cells, and in particular, appropriately selected non-immunogenic encapsulated xenogeneic and/or allogeneic choroid plexus (CP) cell-containing central nervous system implants as described herein, can be induced by being contacted with a choroid plexus inducing agent as provided herein, to produce altered, and in certain preferred embodiments increased, levels of one or more cerebrospinal fluid (CSF) components. The encapsulated xenotransplanted (and/or allotransplanted) choroid plexus cells are surprisingly long-lived following implantation into a central nervous system site (e.g., brain tissue), and preferably contain xenogeneic and/or allogeneic choroid plexus cells obtained from a donor mammal that is substantially free of human pathogens. The present embodiments advantageously increase the potency and efficacy of choroid plexus cell xenotransplants and/or allotransplants while decreasing the number of implantation sites and implanted capsules that are needed; preferred embodiments also reduce the risk of pathogen transmission to recipients.

Accordingly, appropriate selection and induction of choroid plexus cell-containing capsules according to the present disclosure, including the composition and size of the capsules, the source, preparation and number of cells that are contained therein, and the use of a choroid plexus inducing agent that alters and for certain preferred CSF components increases CSF component production by the CP cells, represent new and useful improvements to CP encapsulation for the treatment of nervous system disease, which improvements could not have been predicted from previous knowledge in the art.

As disclosed herein for the first time, after contacting selected encapsulated xenogeneic and/or allogeneic choroid plexus (CP) cells with a choroid plexus inducing agent, one or more CSF components may be produced by such cells at a level that is altered (e.g., increased or decreased in a statistically significant manner relative to the level prior to or in the absence of contact with the CP inducing agent) and which for certain preferred CSF components is greater than the level at which the xenotransplanted and/or allotransplanted CP cells produce the CSF component(s) without being contacted with the choroid plexus inducing agent.

Surprisingly, the effects of inducing such increased (e.g., greater than uninduced levels, in a statistically significant manner) levels of CSF production by selected encapsulated xenogeneic and/or allogeneic choroid plexus (CP) cell-containing implants in the central nervous system (CNS) (or in certain embodiments in the peripheral nervous system (PNS)) may be achieved using CP cells that remain viable in semi-permeable capsules for greater than 2, 3, 4, 5, 6, 7, 8, 9, 10, 11, 12, 13, 14, 15, 16, 18, 20, 24 or more months post-implantation, substantially without elicitation of localized immunological or inflammatory reactions such as immune rejection of the CP cell-containing capsules, host extracellular matrix deposition on the capsules, or a foreign body response to the capsules.

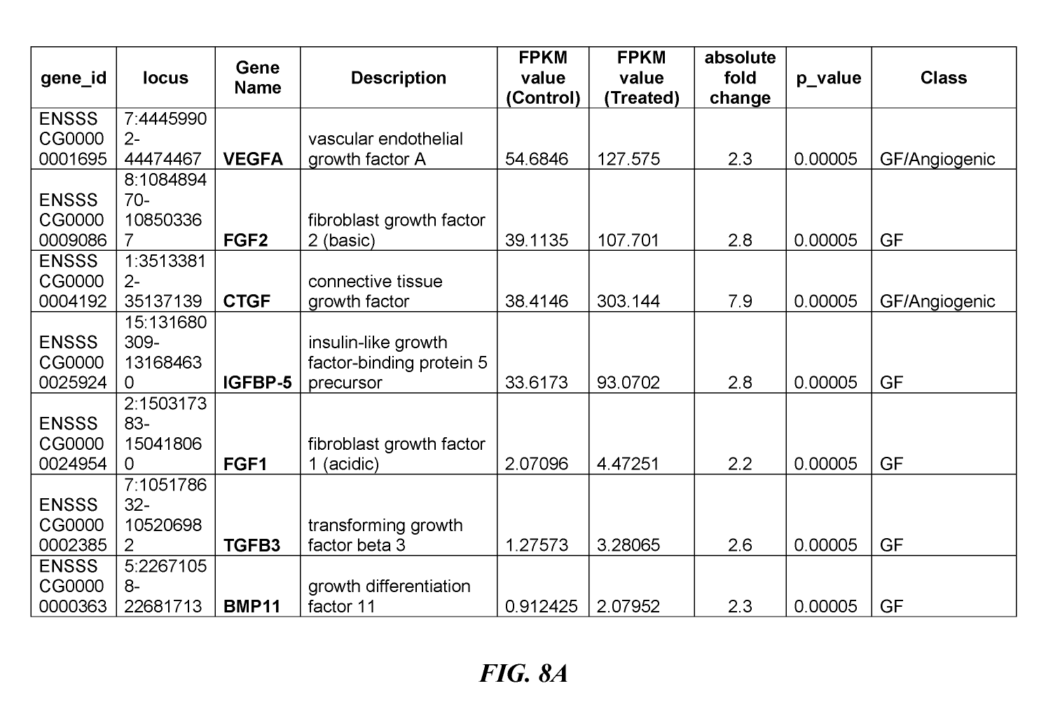

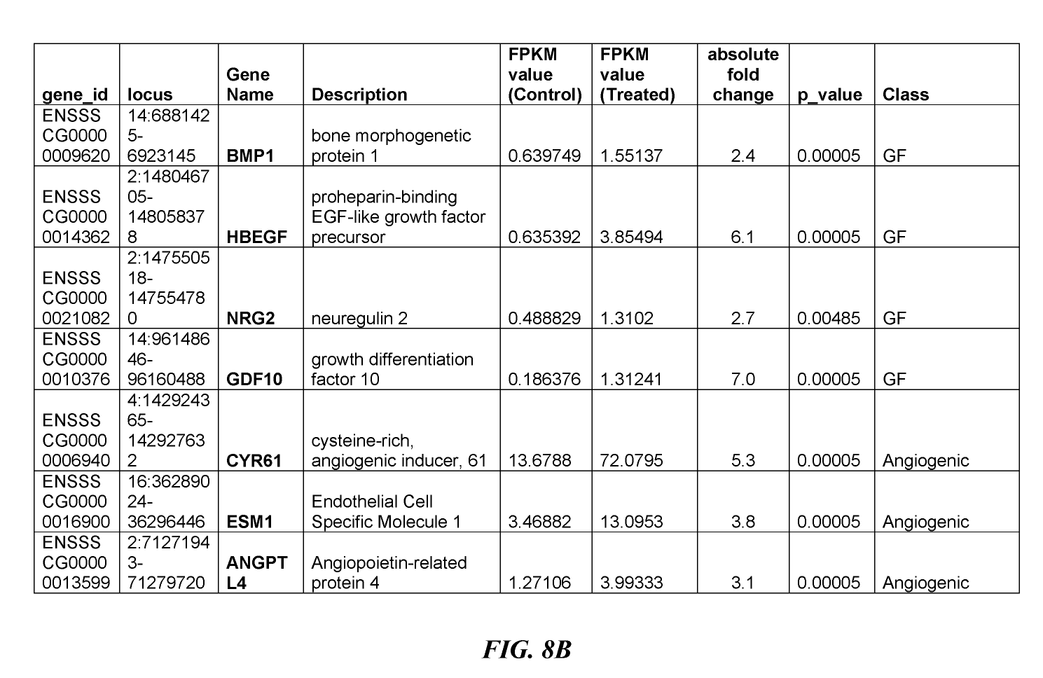

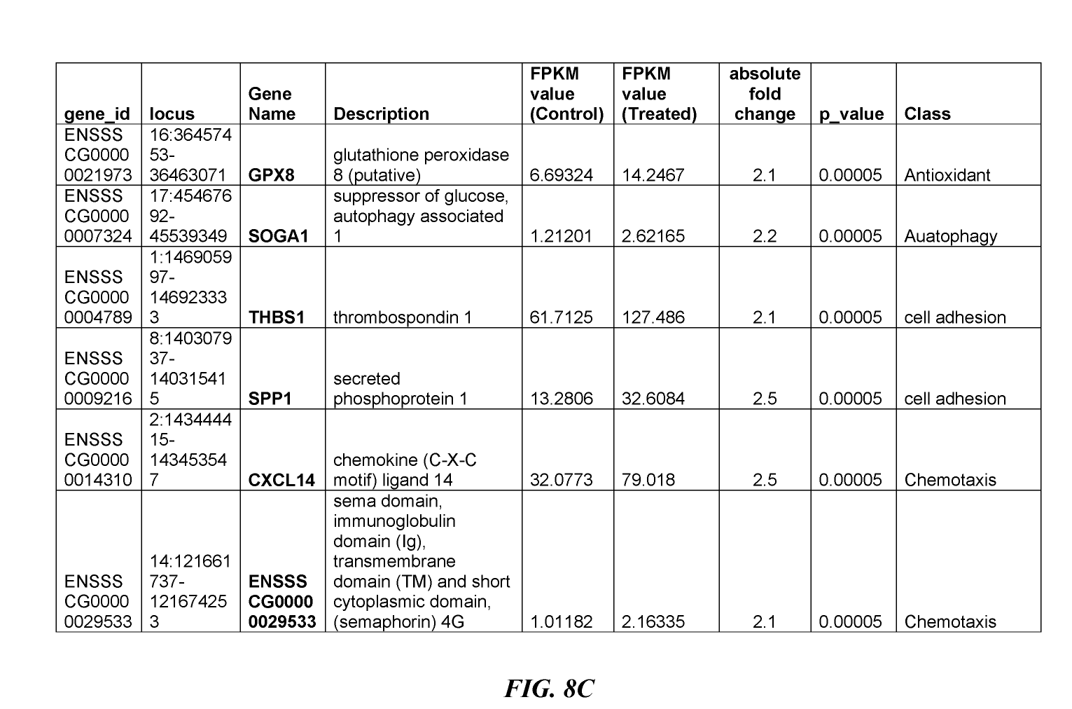

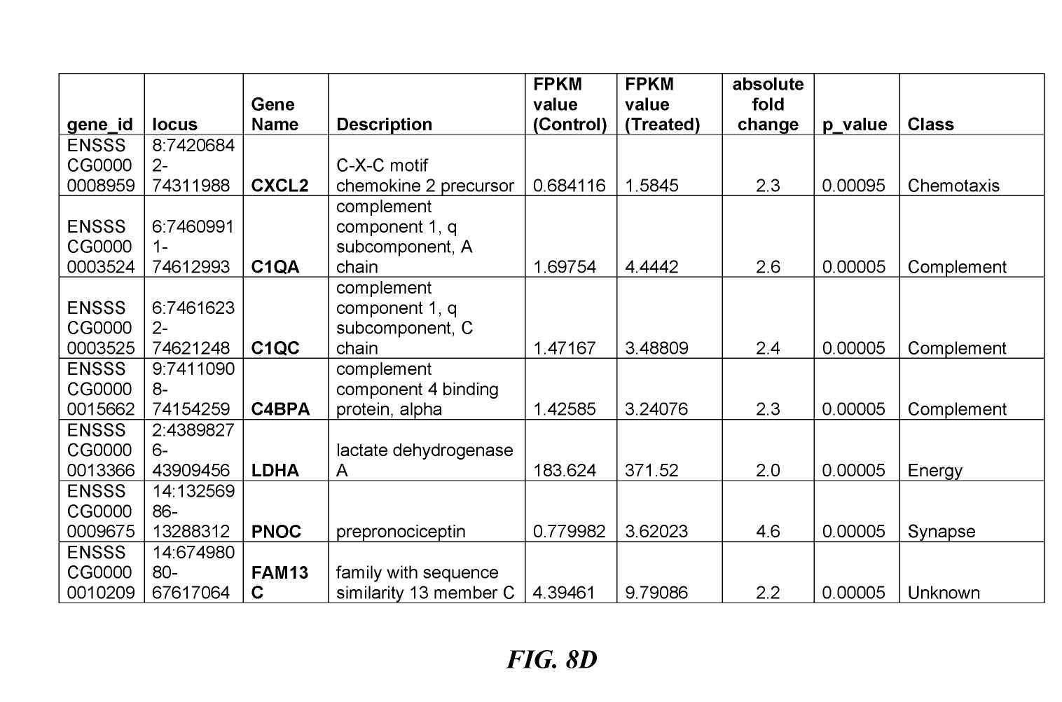

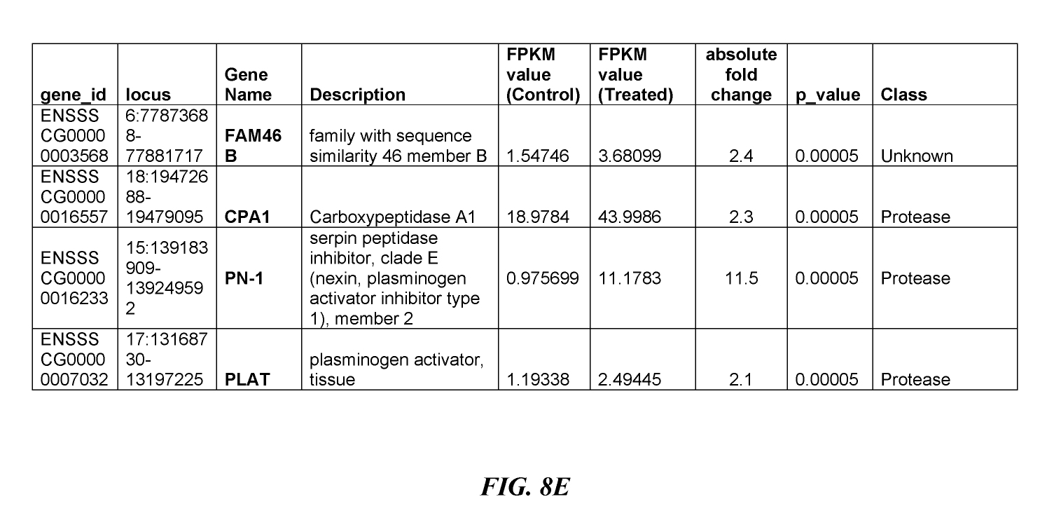

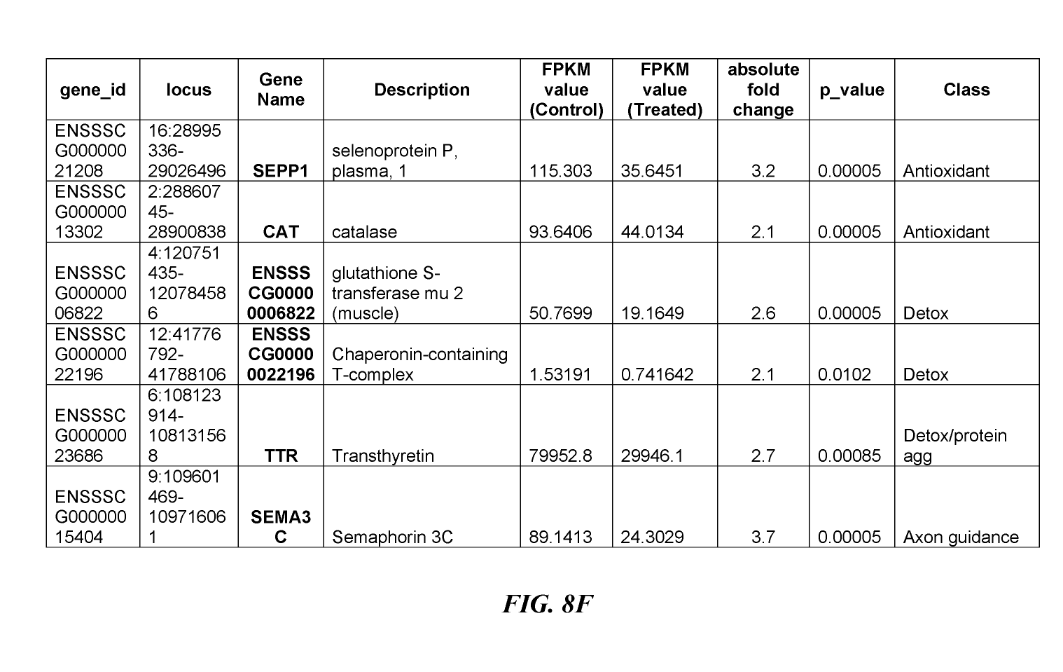

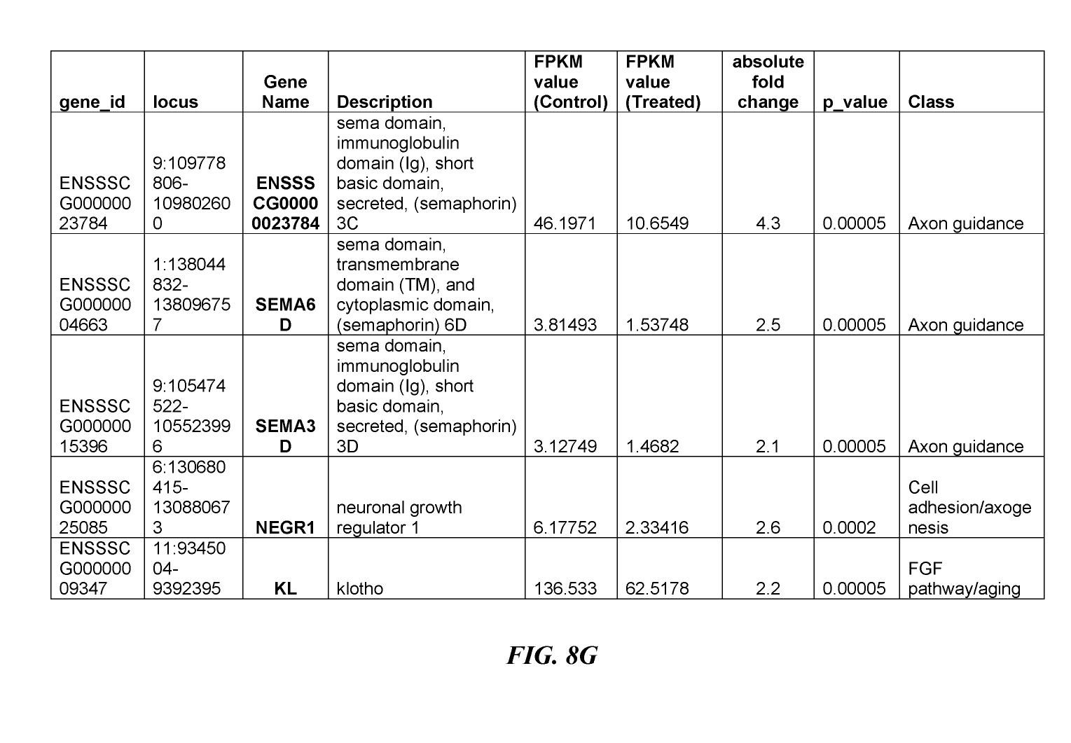

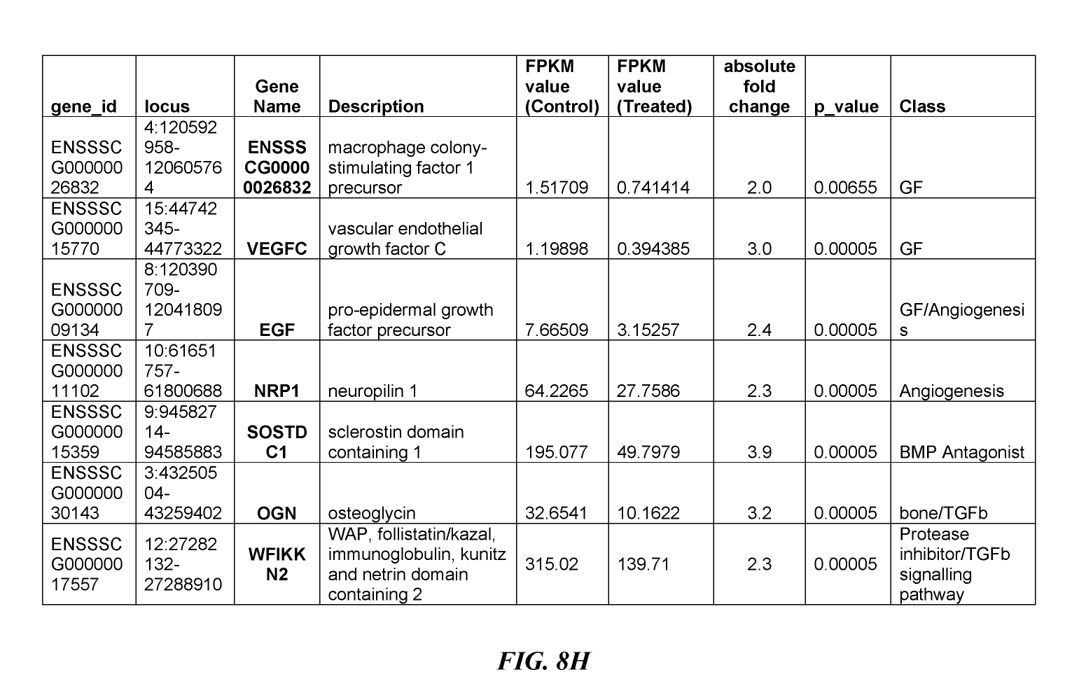

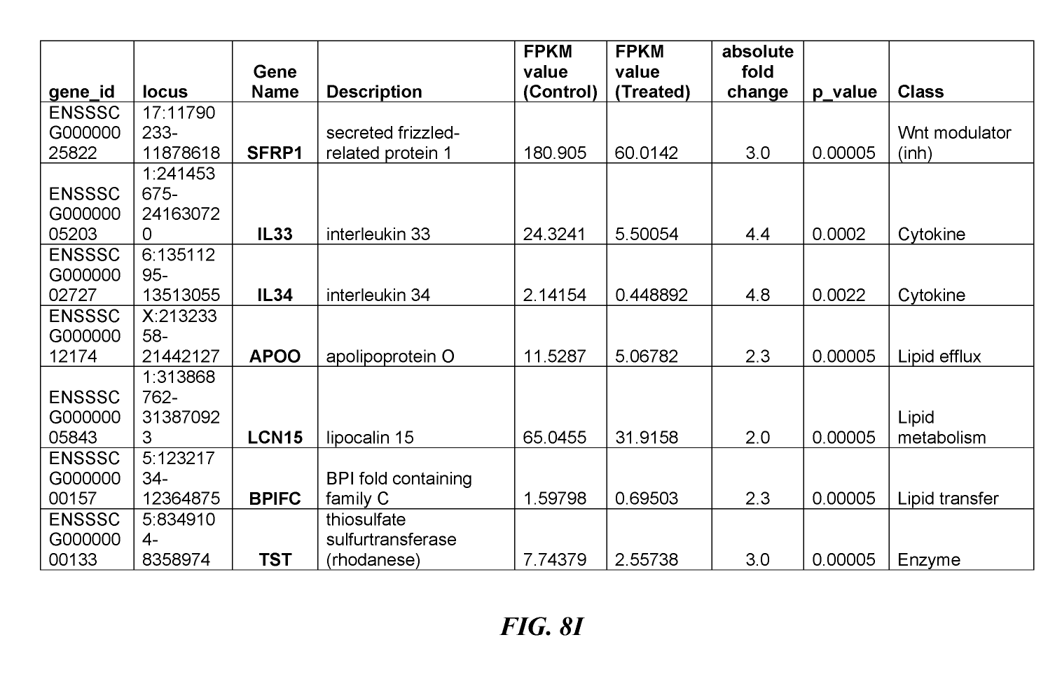

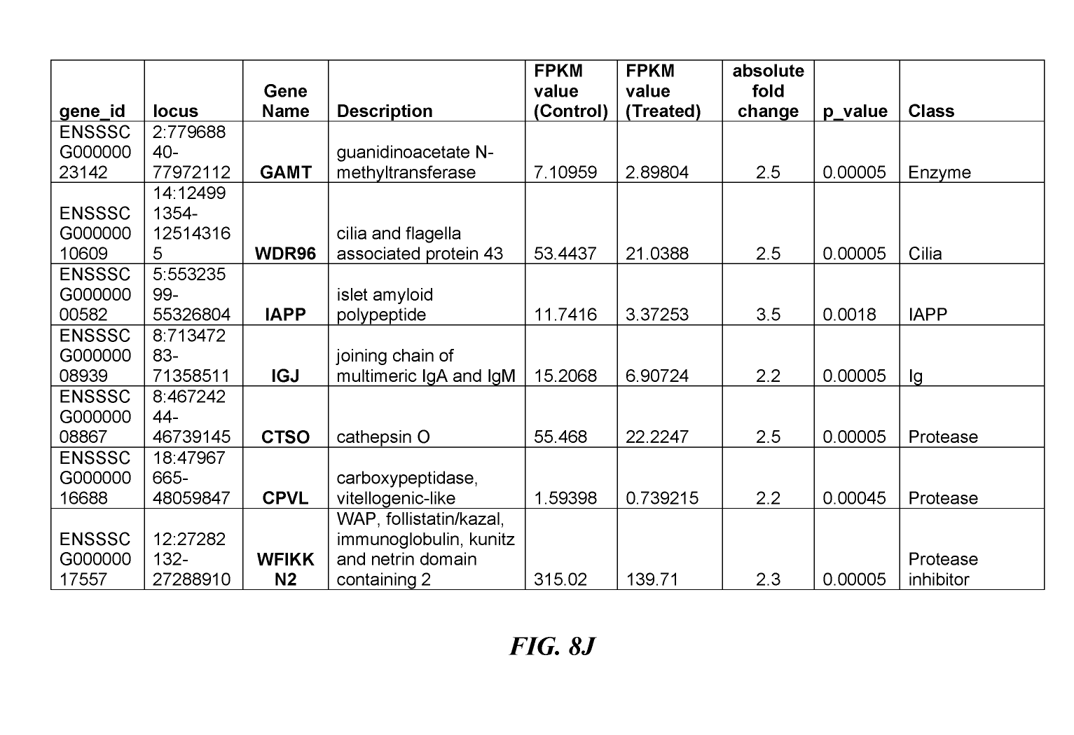

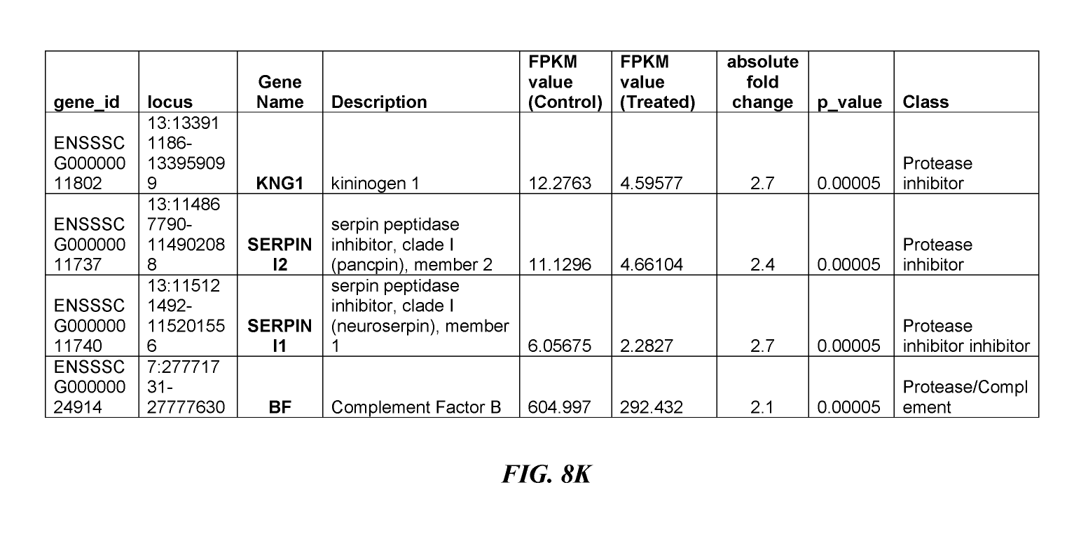

In particular, the present disclosure for the first time describes that the direct effects of a choroid plexus inducing agent as provided herein, on encapsulated CP cells that have been selected as also described herein, alter the expression levels of genes encoding known CSF components (as shown, for example, in FIG. 8), including an increase in the production by CP cells of certain CSF components that have favorable consequences in the CNS, such as neuroprotection. Beneficial effects of increased CSF production may therefore be achieved by employing a choroid plexus inducing agent according to the present disclosure, and can be exploited for use in treating any of a number of nervous system diseases or disorders or other conditions in which altered, and in certain preferred embodiments increased, production of one or more CSF components may be desirable. For example by way of illustration and not limitation, the herein disclosed embodiments may be used to treat certain CNS disorders that are characterized by below-normal, inadequate or suboptimal levels of one or more CSF components and/or by deficits in one or more CNS activities, such as may be due to aging processes, exposure to toxins, or disease, and that may be at least partially restored by increased CSF production.

CP Inducing Agents

Accordingly and in certain embodiments, despite the general understanding in the art that CSF is constitutively produced in the CNS, the present disclosure relates in part to the surprising discovery that CP inducing agents as described herein can induce CP cells to increase the production of certain CSF components and may also induce decreased production of certain other CSF components (e.g., FIG. 8). Without intending to be bound by theory, it is therefore believed that by acting on CP cells to promote higher production levels of certain CSF components such as neuroprotective components, the present CP inducing agents permit implantation of fewer encapsulated xenotransplanted and/or allotransplanted CP cells than had been previously believed to be feasible to achieve a therapeutically effective amount of such CP cells. Further according to non-limiting theory, because the induced CP cell xenotransplants and/or allotransplants produce CSF components, the present methods provide unprecedented efficiency and safety by avoiding the need for a greater number of implanted capsules at a greater number of CNS implantation sites, and also by avoiding the need for systemic administration of higher doses of a CP inducing agent the effects of which might otherwise be counteracted or diluted through its interaction with other cell types.

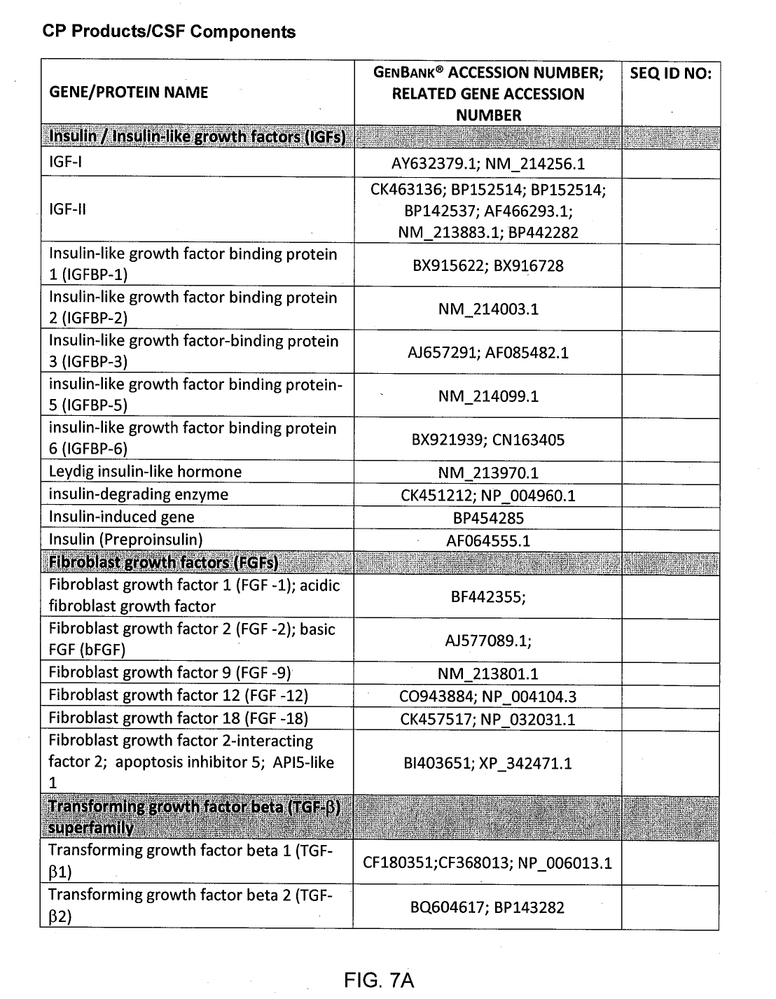

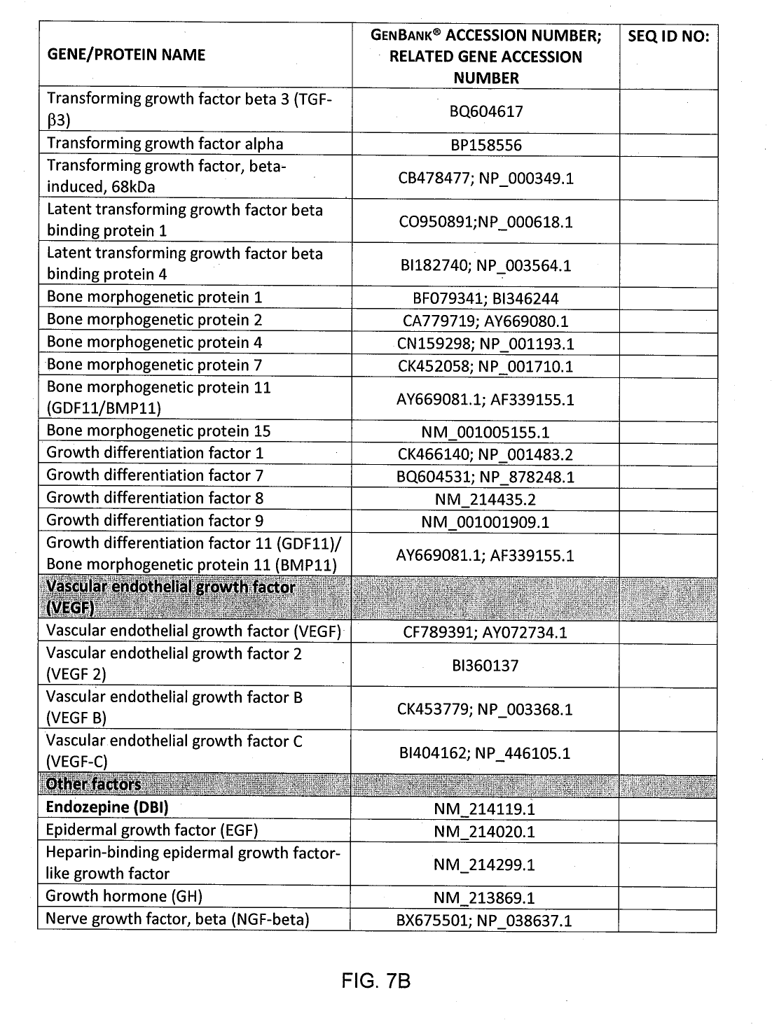

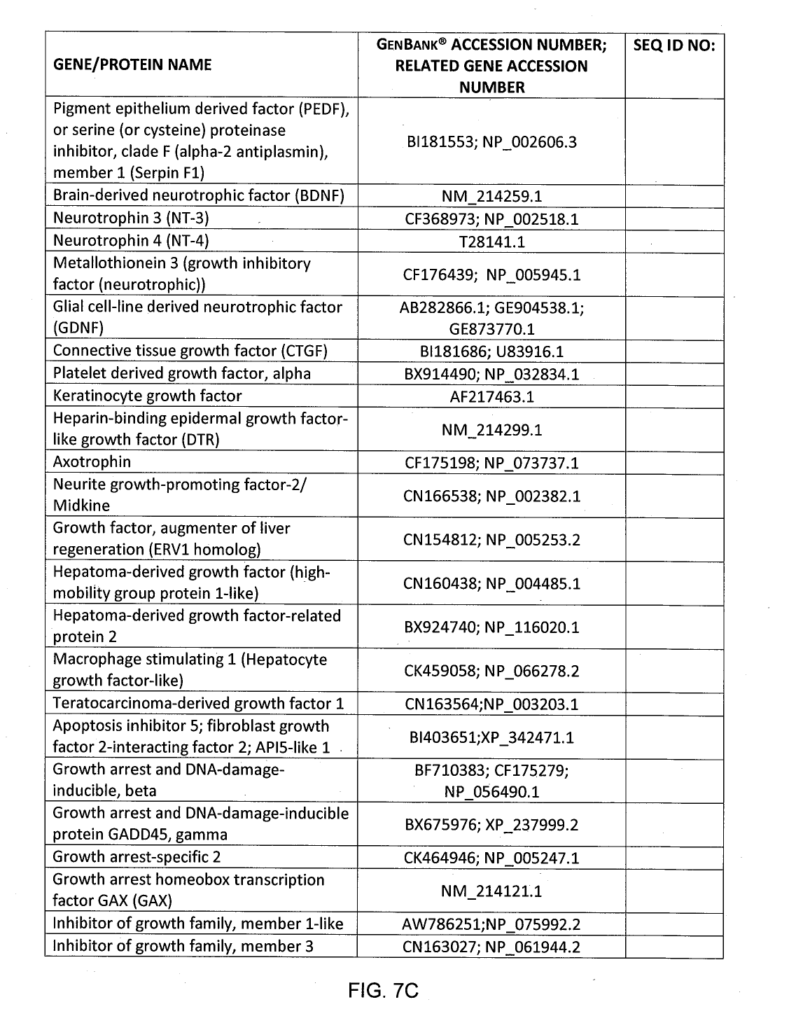

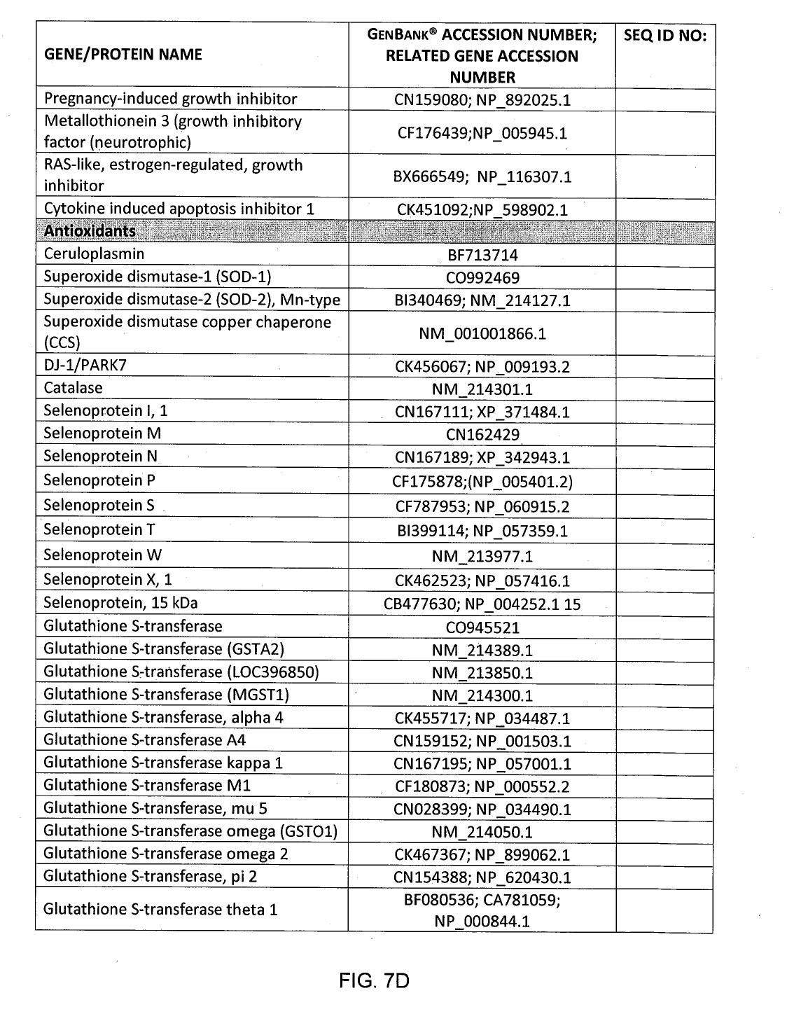

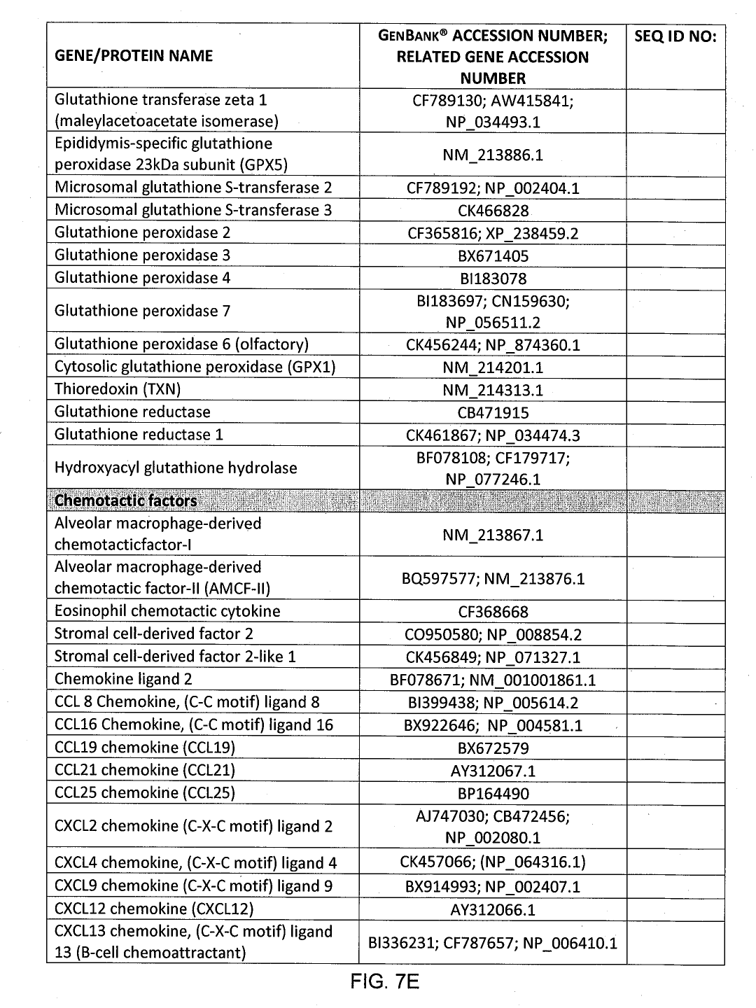

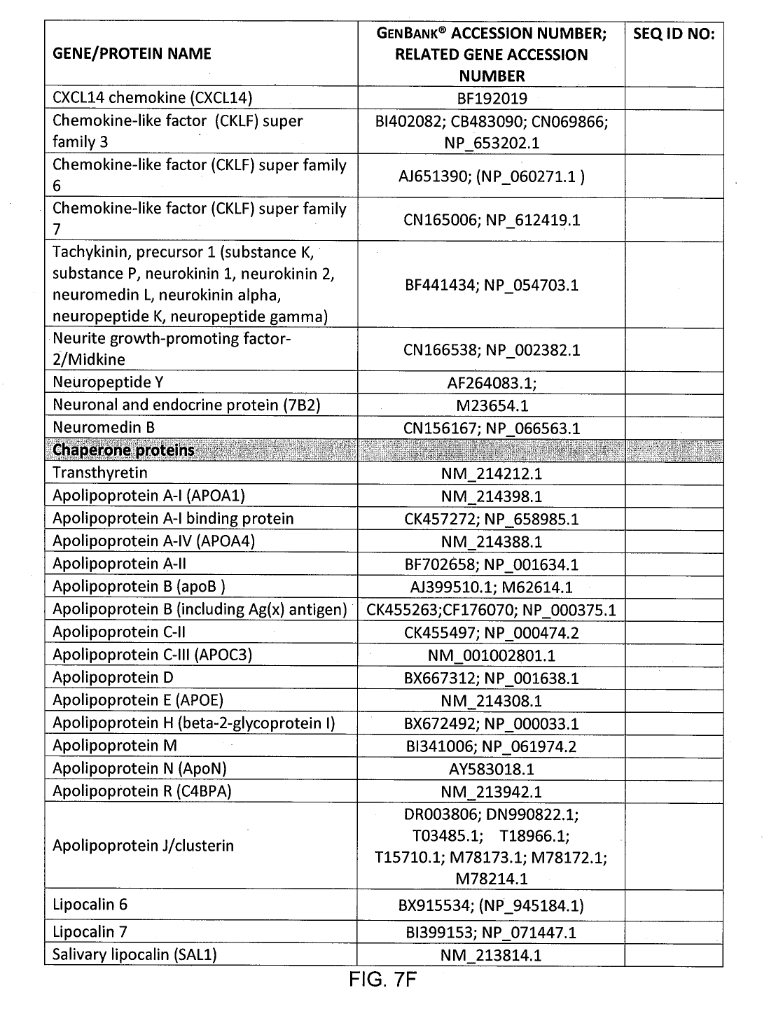

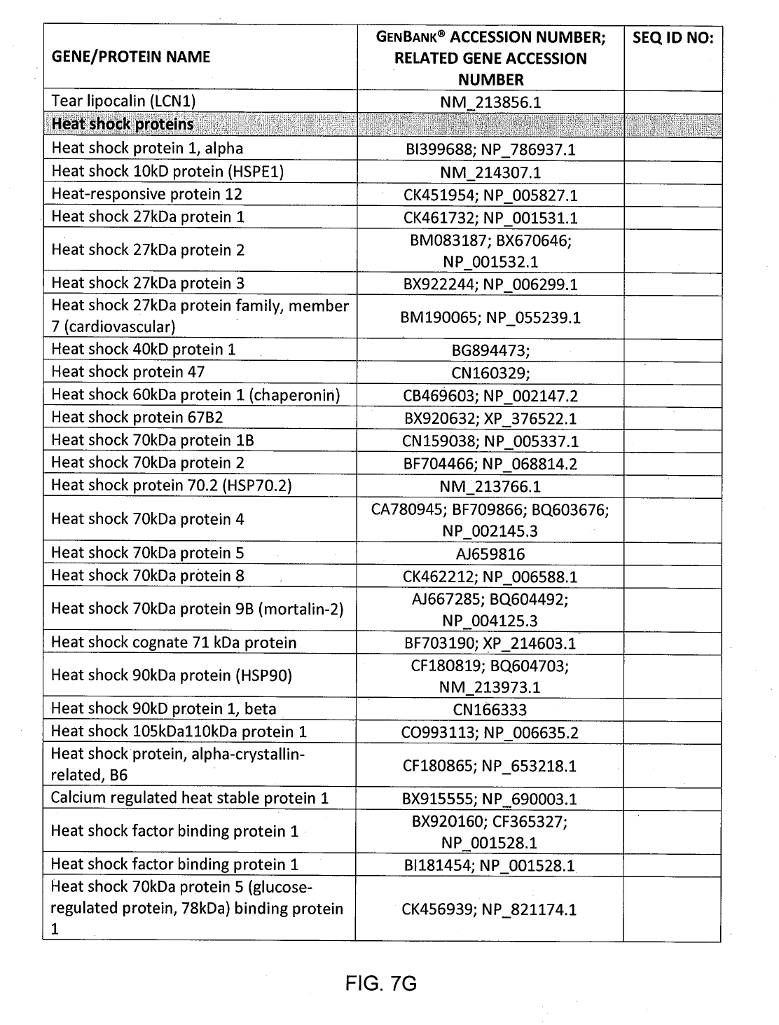

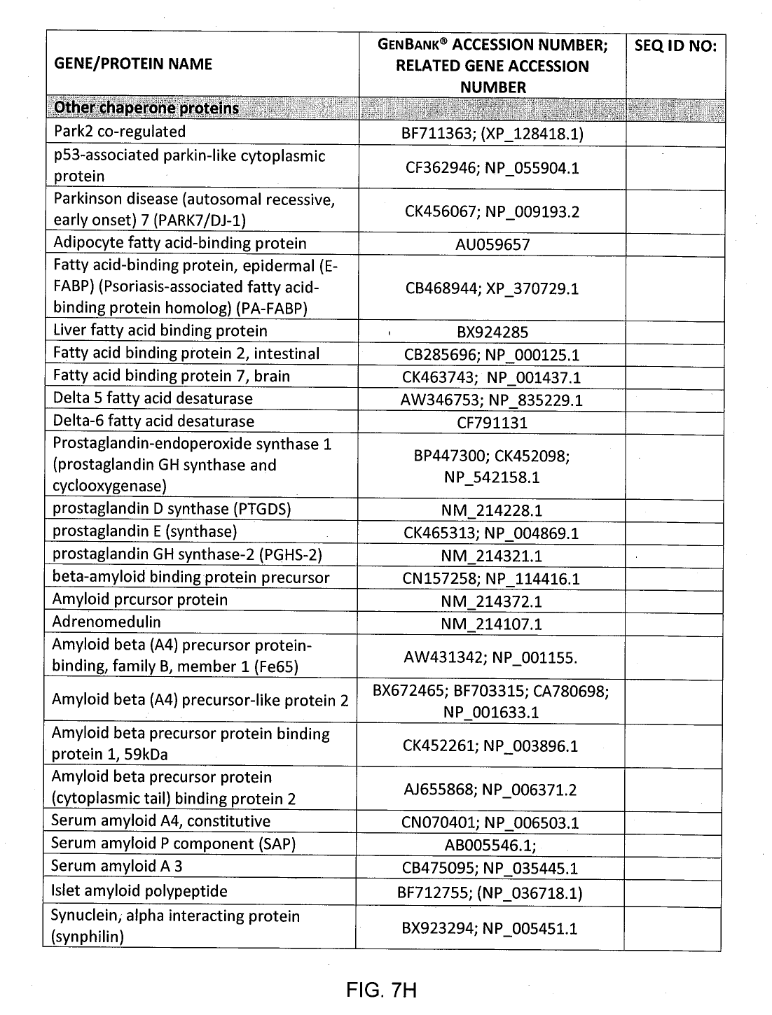

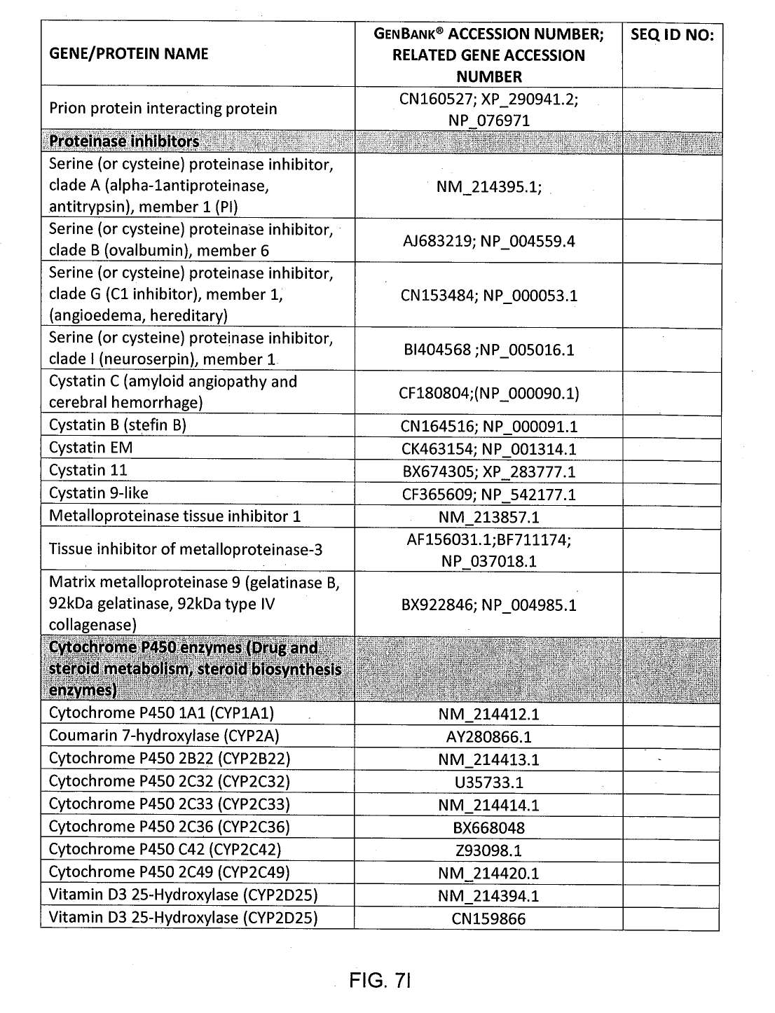

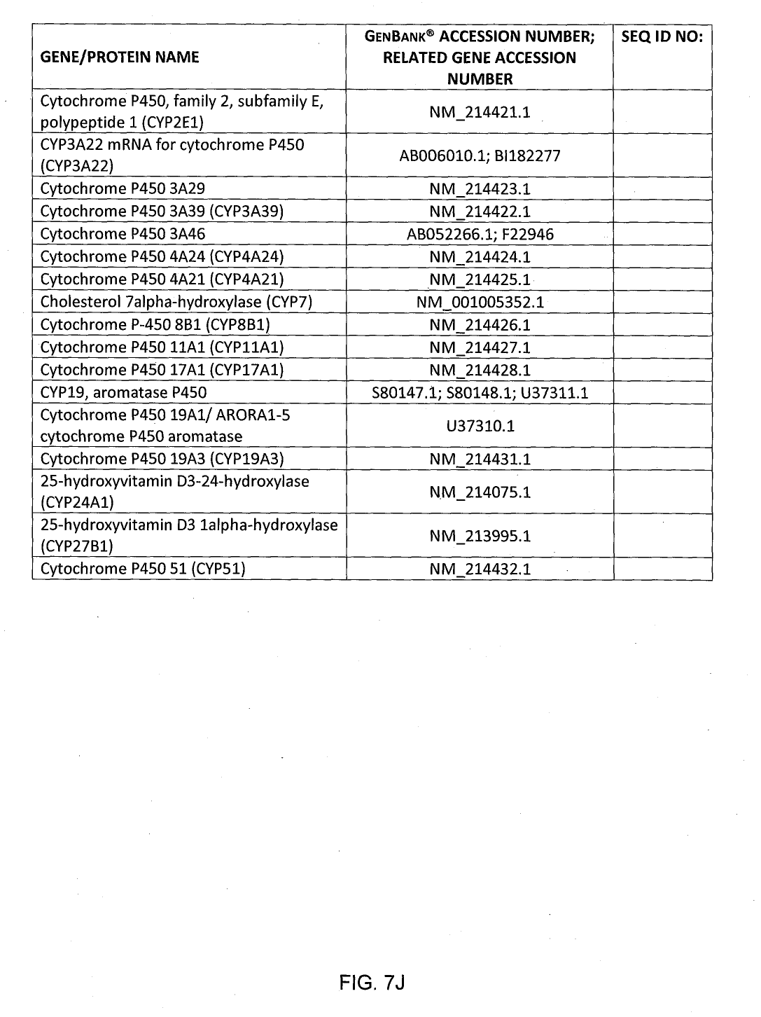

Additionally, according to the present disclosure, agents that may previously have been believed to have neuroprotective effects through their action directly on neurons are here unexpectedly shown to act advantageously as CP inducing agents. A CP inducing agent as provided herein includes any agent that, when contacted with a CP tissue cell, is capable of inducing CP cells to alter (e.g., increase or decrease in a statistically significant manner relative to a control situation when the CP inducing agent has not been introduced), and in certain preferred embodiments, to increase CSF component production, i.e., by inducing the CP cells to produce increased levels (e.g., levels that are greater in a statistically significant manner than those produced without introducing the CP inducing agent) of one or more CSF components. The CSF components (e.g., representative examples of which are disclosed in FIGS. 7 and 8) may then confer protective effects on neurons and/or may yield other beneficial effects for treating nervous system diseases. The presently contemplated embodiments are not intended to be so limited, however, such that there are also contemplated embodiments in which contacting CP cells with a CP inducing agent may induce CP cells to decrease CSF component production, i.e., by inducing the CP cells to produce decreased levels (e.g., levels that are lower in a statistically significant manner than those produced without introducing the CP inducing agent) of one or more CSF components and thereby confer a clinical benefit to a subject undergoing treatment.

For example, and as described in greater detail elsewhere herein including in the Examples (e.g., FIG. 8), the alkali metal lithium is shown herein for the first time to be a choroid plexus inducing agent that induces CP cells to produce certain CSF components at a level that is increased (FIG. 8A-E) relative to the level at which the CP cells produce the CSF components prior to being contacted with lithium (e.g., as lithium chloride (LiCl) or lithium carbonate), whilst also inducing decreased (FIG. 8F-K) expression of certain other CSF components relative to the level at which the CP cells produce the CSF components prior to being contacted with lithium.

Lithium has long been regarded as an agent that confers neuroprotective effects, although its site and mechanism of action are not fully understood. By way of non-limiting theory, lithium is believed to act at least in part as an inhibitor of glycogen synthase kinase-3beta (GSK3β) and by indirectly inhibiting N-methyl-D-aspartate (NMDA)-receptor-mediated calcium influx in neurons (e.g., Chiu et al., 2011 Zhong Nan Da Xue Xue Bao Yi Xue Ban 36(6):461 (PMID 21743136); Chiu et al., 2010 Pharmacol. Ther. 128:281; Rowe et al., 2004 Expert Rev. Mol. Med. 6:1), from which neuroprotective effects underlying its current clinical use in treating bipolar mood disorder as well as its proposed uses for treating a variety of CNS injuries and neurodegenerative diseases have been contemplated (Id.).

Lithium activity in the CNS is associated with its effects on neurons and on CNS electrolyte transport, including electrolyte transport by the choroid plexus (CP), but the influence of lithium on enhanced production of CSF components by CP has not been recognized prior to the effects disclosed for the first time herein. For instance, Pulford et al. (2006 Neuropsychiatr. Dis. Treat. 2(4):549) described lithium-induced down-regulation in rat CP of transthyretin, a major CSF component, mimicking the decreased transthyretin levels that have been detected in clinical depression. From such observations, and in view of the general lack of understanding of the neuroprotective mechanisms of lithium or of CP regulation, however, the presently described lithium-induced increase in the production of certain CSF components by CP cells as disclosed herein would not have been expected.

Certain embodiments thus contemplate a CP inducing agent that may comprise any suitable lithium salt, i.e., a lithium compound that comprises cationic lithium and that can be contacted with cells with no or minimal toxicity, for example, lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or any other lithium salt as may be known to those skilled in the relevant art.

Similarly, other CP inducing agents as provided herein surprisingly induce CP cells to produce increased levels of CSF components and may be employed according to certain embodiments to enhance the potency of the encapsulated CP implants described herein. Contemplated CP inducing agents include Wnt signaling pathway agonists, beta-catenin activators, antioxidants, and 1,25-dihydroxyvitamin D₃. Wnt signaling pathway agonists are known in the art and include, for example, WAY-316606 (Bodine et al., 2009 Bone 44:1063; SFRP inhibitor, 5-(Phenylsulfonyl)-N-4-piperidinyl-2-(trifluoromethyl)benzene sulfonamide hydrochloride, Cat. No. 4767 available from Tocris Bioscience, Bristol, UK), IQ1 (Miyabayashi et al., 2007 Proc. Nat. Acad. Sci. USA 104:5668; PP2A activator), QS11 (Zhang et al., 2007 Proc. Nat. Acad. Sci. USA 104:7444; ARFGAP1 activator), (hetero)arylpyrimidine, or 2-amino-4-[3,4-(methylenedioxy) benzyl-amino]-6-(3-methoxyphenyl) pyrimidine, Norrin (e.g., Ohlmann et al., 2012 Prog. Retin Eye Res. 31:243; Rey et al., 2010 Dev. Dyn 239:102; erratum 2010 Dev. Dyn. 239:1034; GenBank Acc. No. NM_000266), R-spondin-1 (e.g., Peng et al., 013 Cell Rep. 3:1885; GenBank Acc. No. NM_001038633; NM_001242910.1), R-spondin-2 (e.g., GenBank Acc. No. NM_178565; NM_178565.4; NM 001282863.1), R-spondin-3 (e.g., GenBank Acc. No. NM_032784), and R-spondin-4 (e.g., GenBank Acc. No. NM_001029871.3). See also, e.g., Dodge et al., 2011 Ann. Rev. Pharmacol. Toxicol. 51:289; Chen et al., 2010 Am. J. Physiol. Gastrointest. Liv. Physiol. 299:G293; Barker et al., 2006 Nat. Rev. Drug Discov. 5:997; Meijer et al., 2004 Trends Pharmacol. Sci. 25:471; website of laboratory of Dr. R. Nusse, Stanford Univ., Palo Alto, Calif. at the URL: web.stanford.edu /group/nusselab/cgi-bin/wnt/smallmolecules.

In certain embodiments the choroid plexus inducing agent (CP inducing agent) may be a GSK3β inhibitor, for example a lithium salt such as lithium chloride or lithium carbonate, or any suitable lithium salt, i.e., a lithium compound that comprises cationic lithium and that can be contacted with cells with no or minimal toxicity, for example, lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or any other lithium salt as may be known to those skilled in the relevant art.

In certain embodiments the choroid plexus inducing agent (CP inducing agent) may be another GSK3β inhibitor such as SB-216763 (Coghlan et al., 2000 Chem. Biol. 7:793), or BIO (6-bromoindirubin-3′-oxime; Sato et al., 2004 Nat. Med. 10:55).

Included in certain contemplated embodiments, but expressly excluded from certain other contemplated embodiments, the choroid plexus inducing agent (CP inducing agent) may be an antioxidant such as mitoquinol (e.g., 10-(6′-ubiquinoyl) decyltriphenylphosphonium salt, Nierobisz et al., 2010 Comp Biochem Physiol B Biochem Mol Biol. 158(2):125, available under the trademark MITOQ® from Antipodean Pharmaceuticals, Inc., Auckland, NZ, or any other mitochondrially targeted antioxidant disclosed in WO/05/019232, U.S. Pat. No. 7,888,335, WO/05/019233, or U.S. Pat. No. 7,888,334), ubiquinol (coenzyme Q, Wang et al., 2013 Crit Rev Biochem Mol Biol. 48(1):69), tocopherols, tocotrienol (vitamin E, Traber et al., 2011 Free Radic Biol Med. 51(5):1000), α-tocopherol, γ-tocopherol, 2-aminoethanesulfonic acid (taurine), ascorbic acid, glutathione, or melatonin.

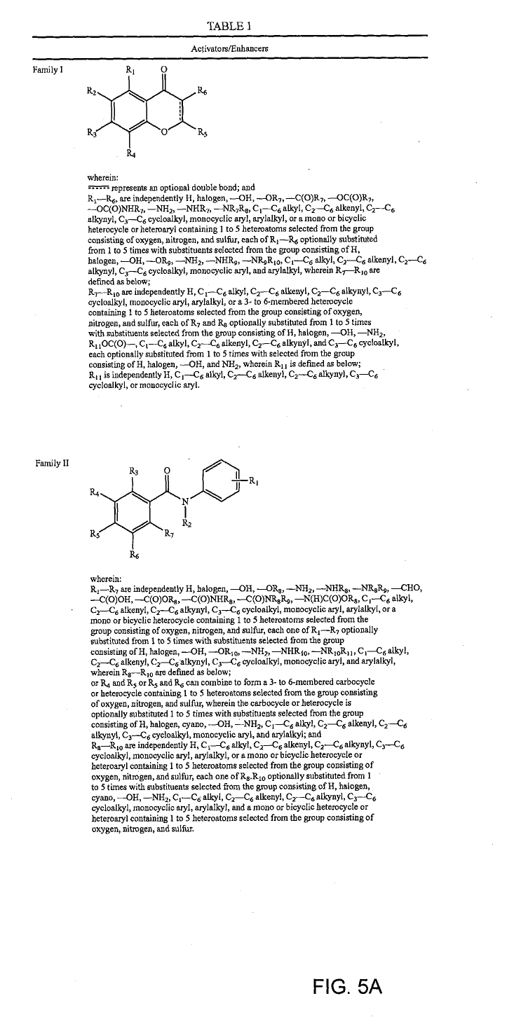

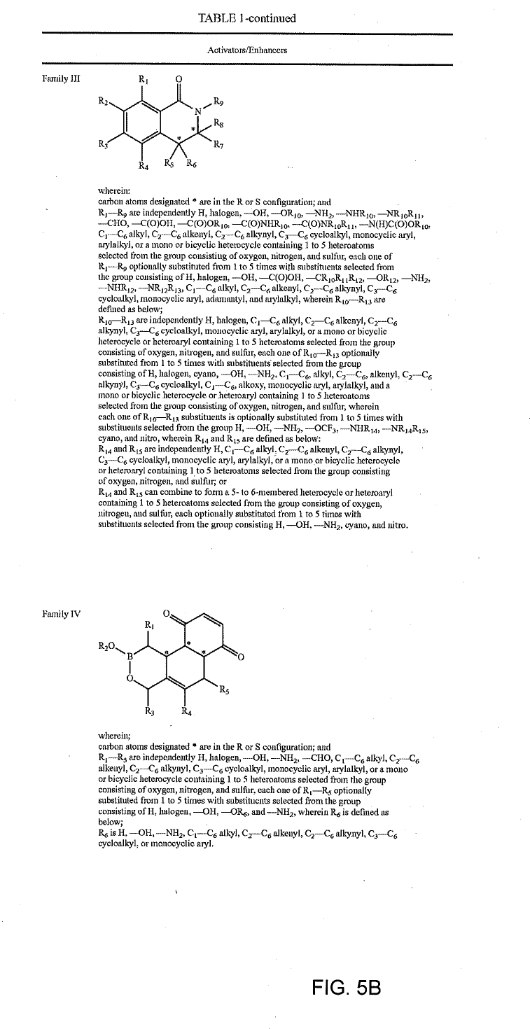

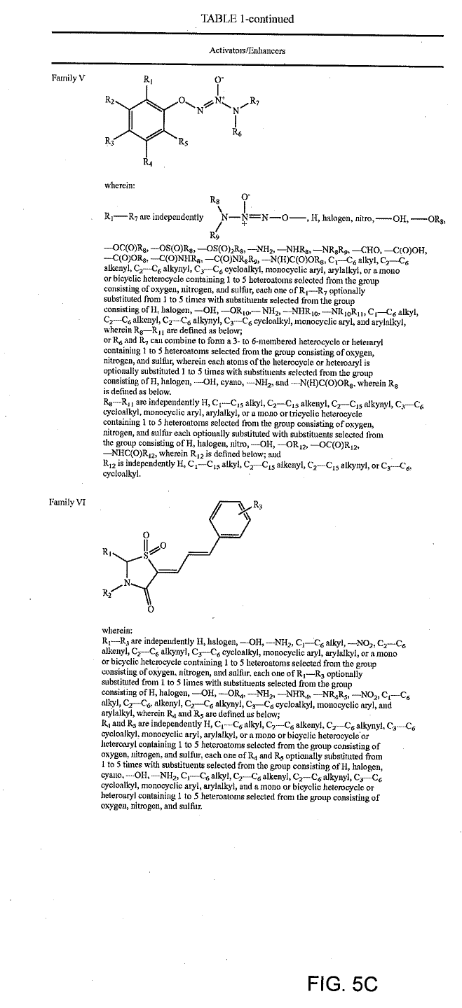

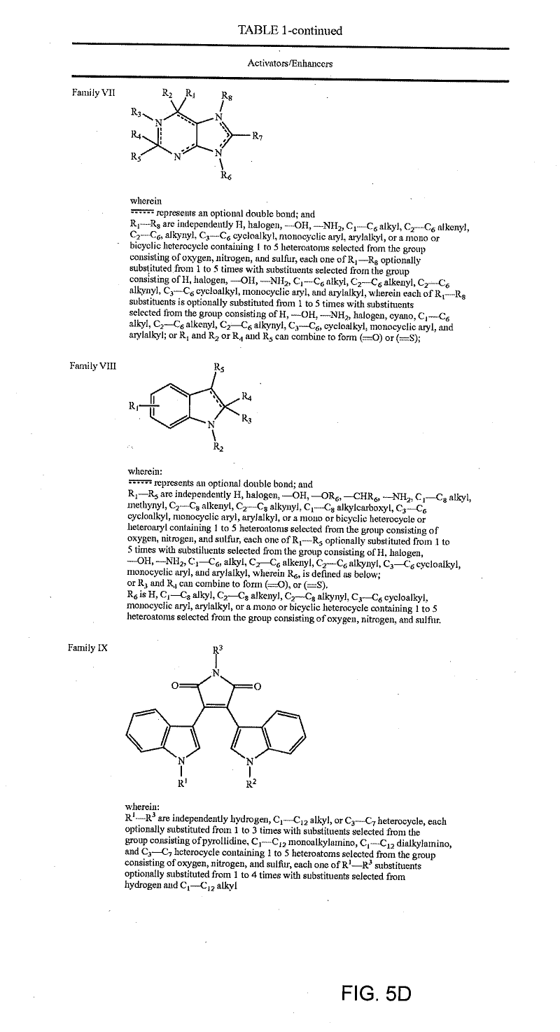

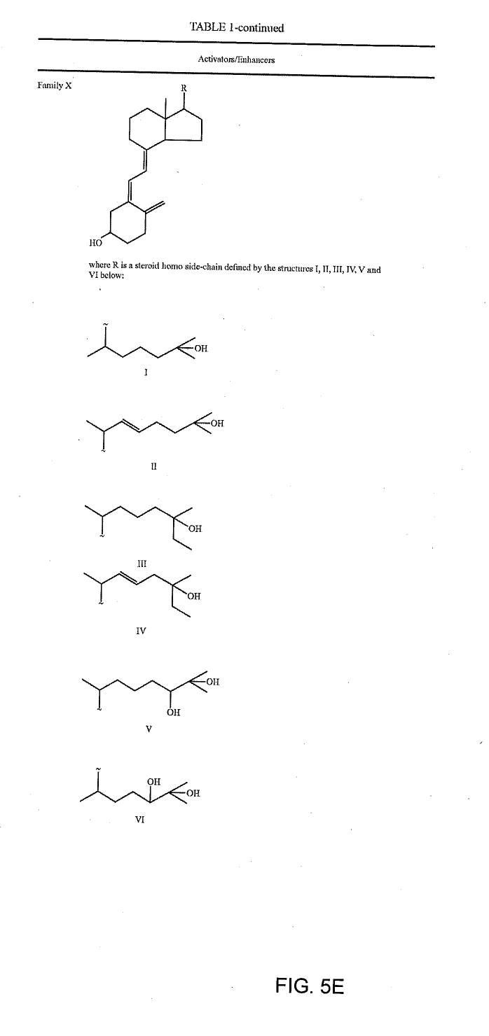

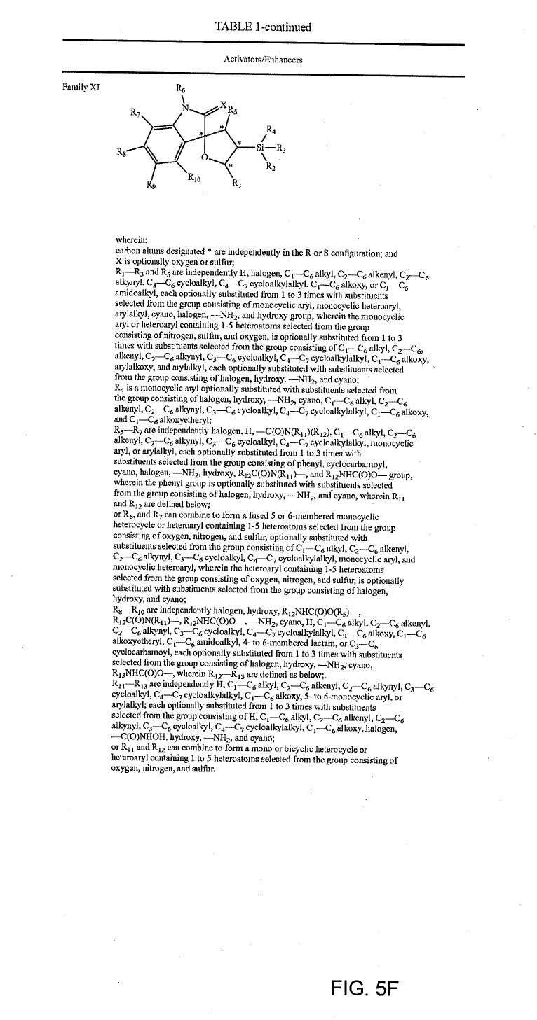

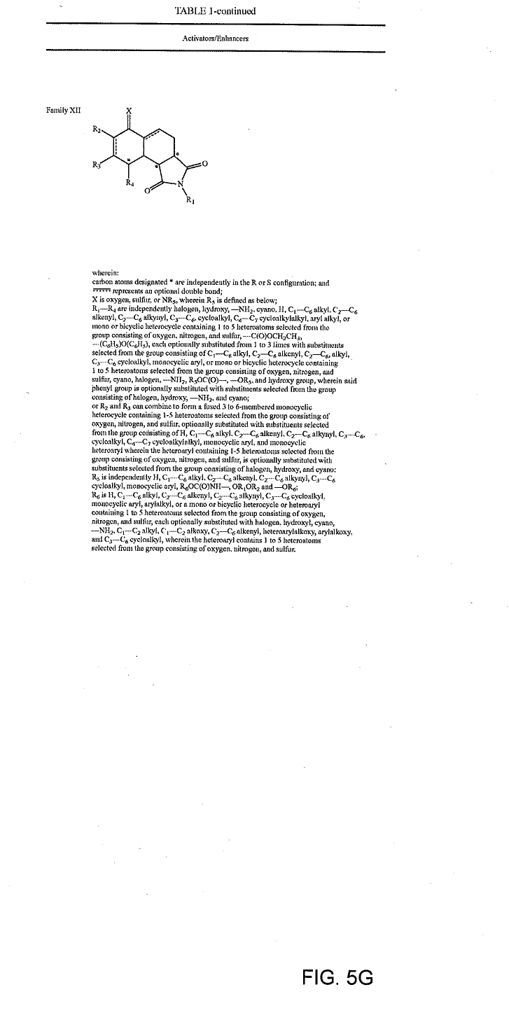

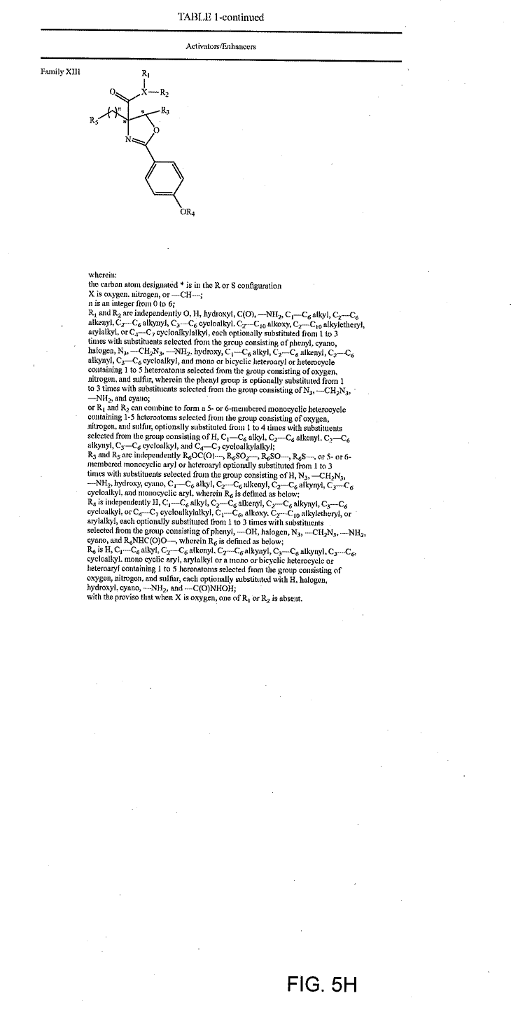

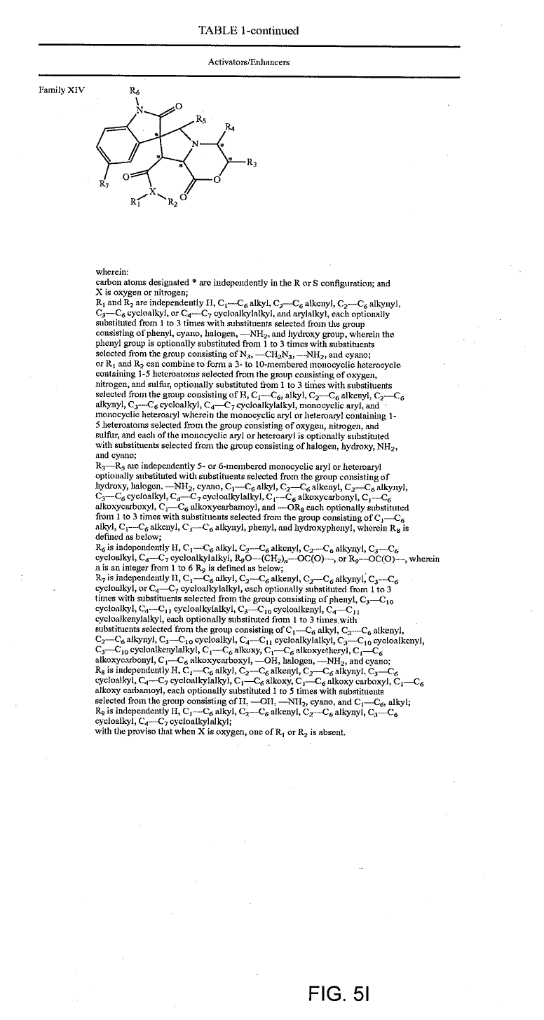

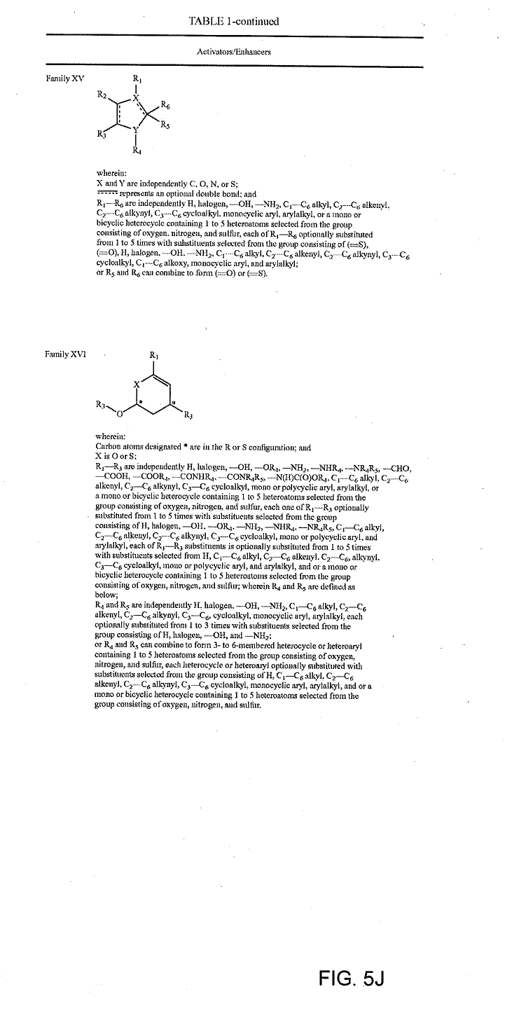

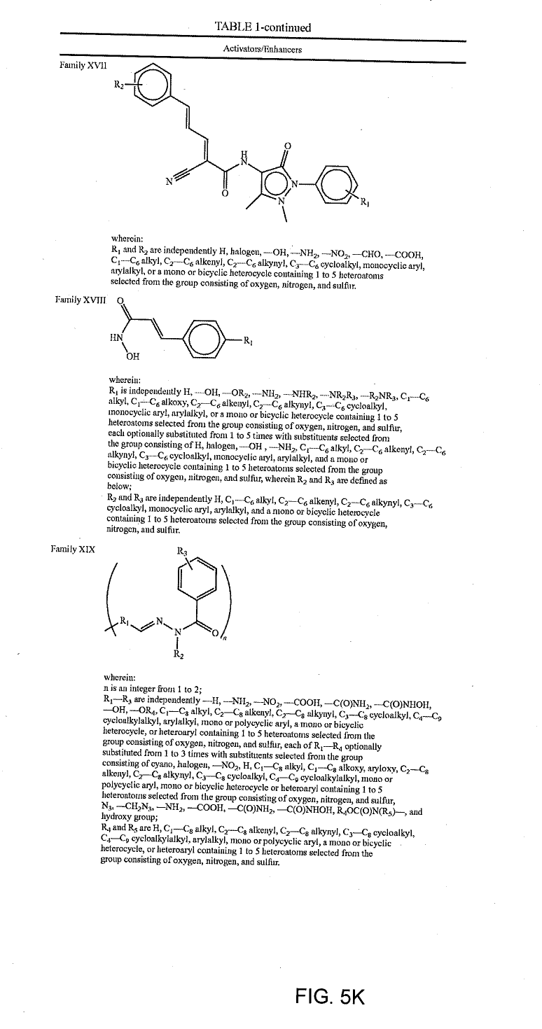

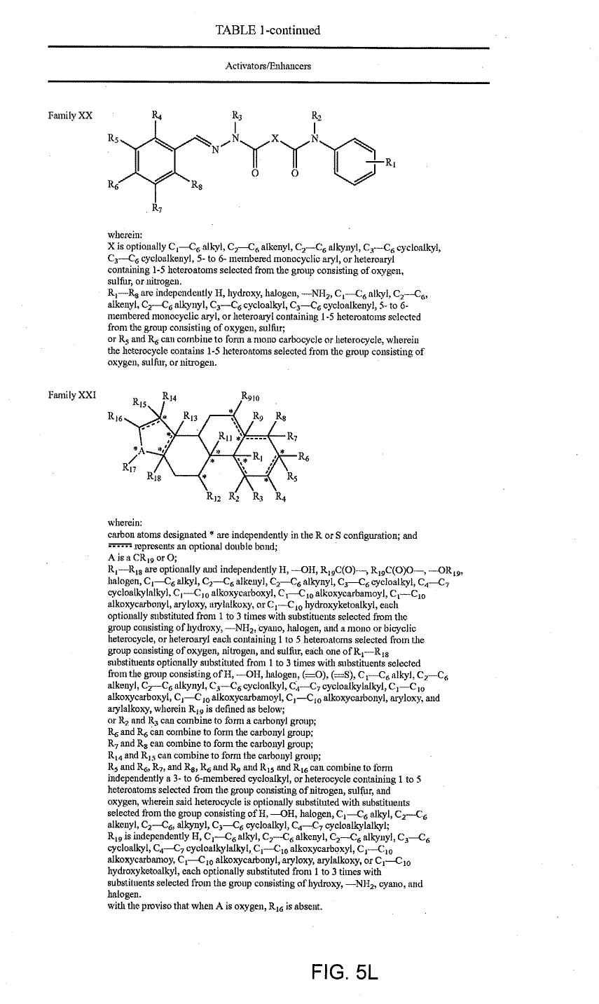

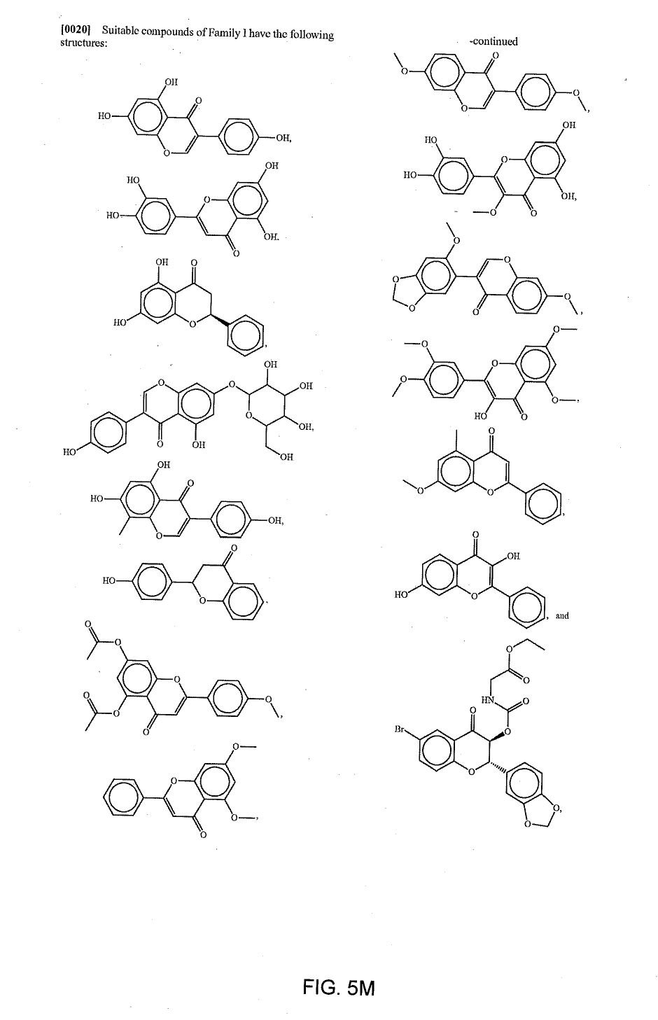

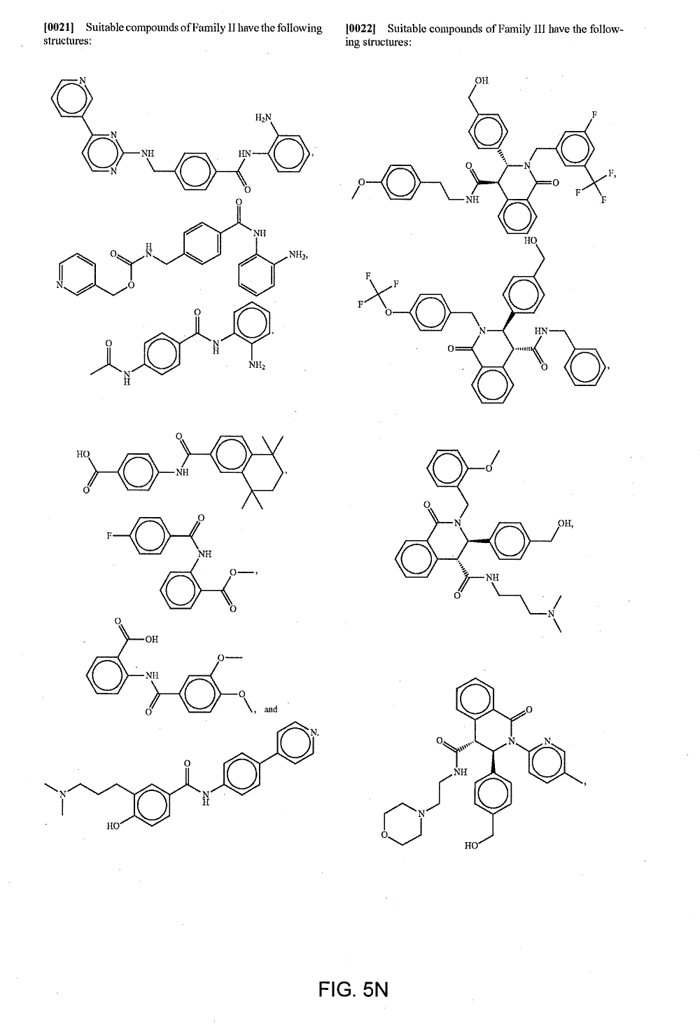

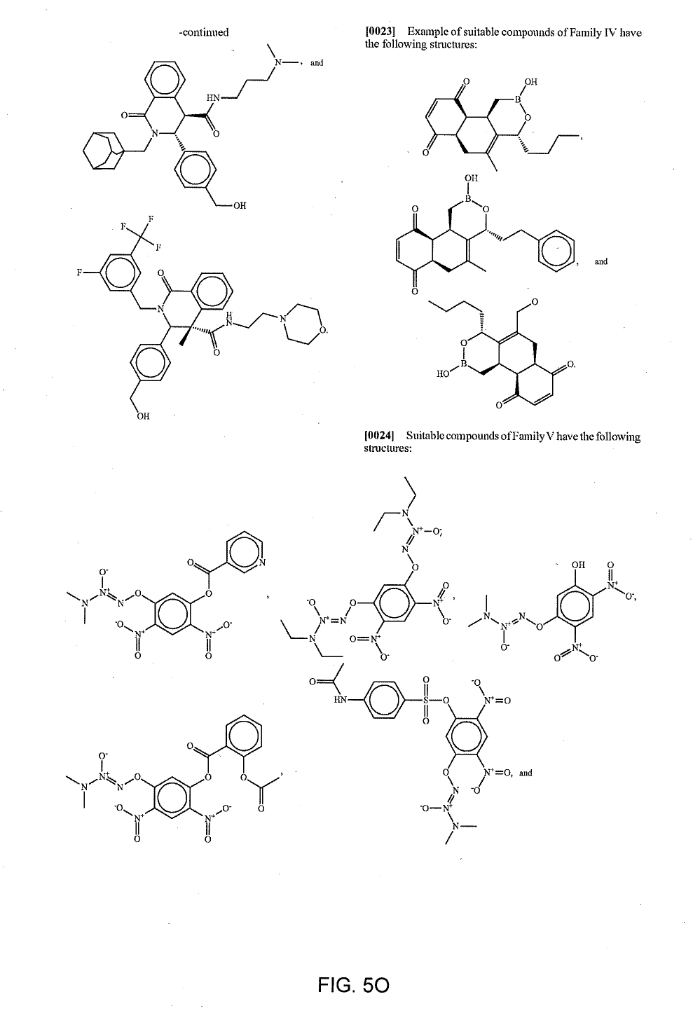

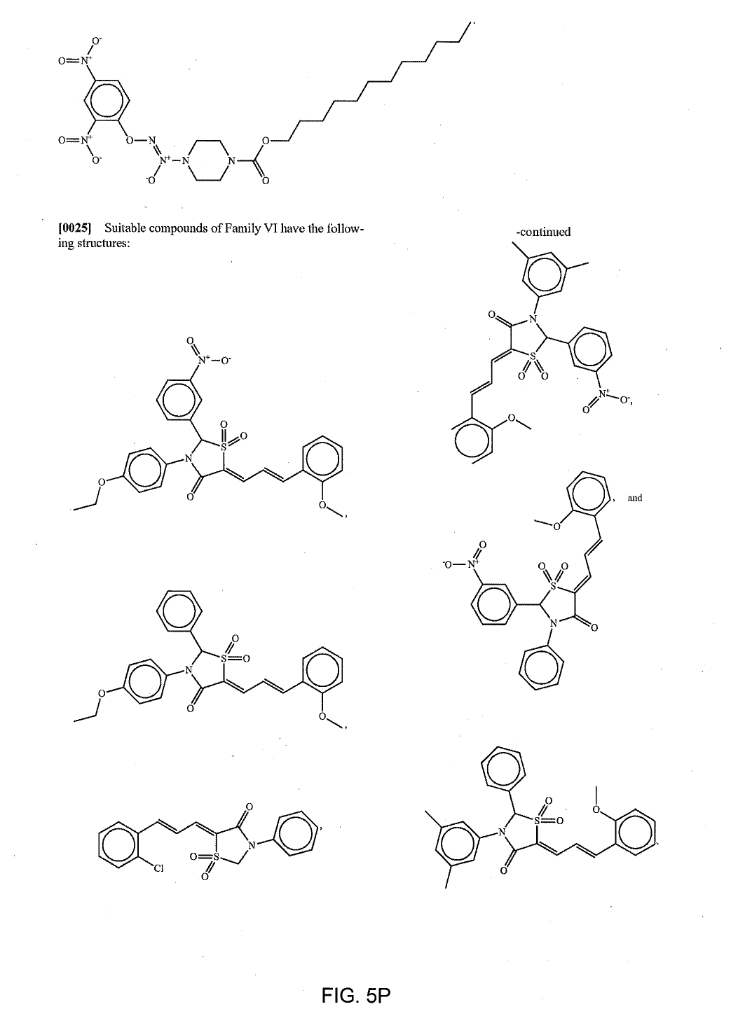

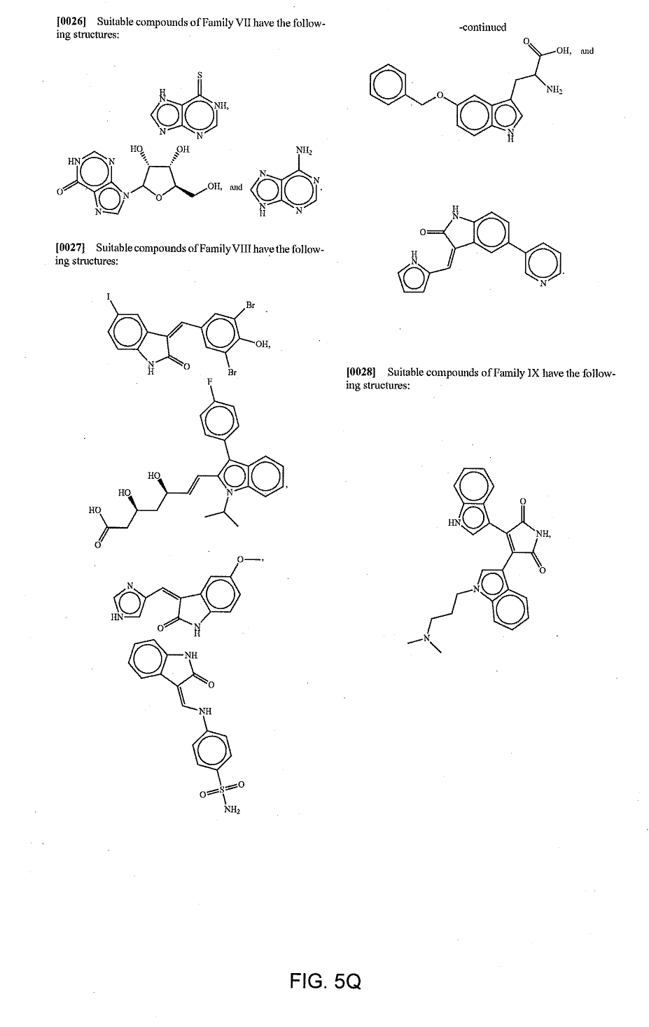

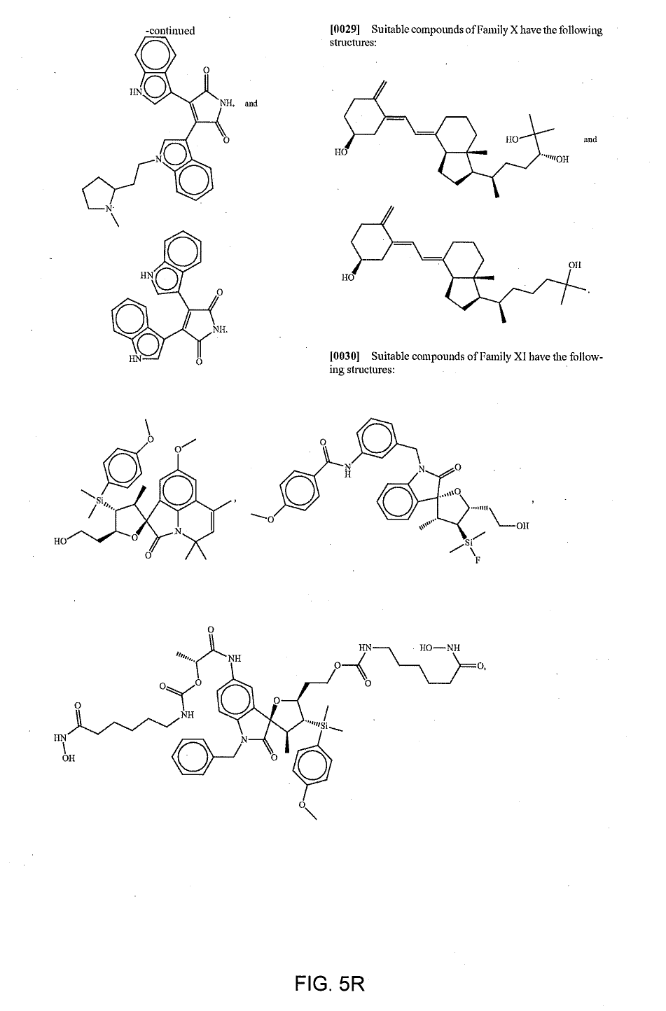

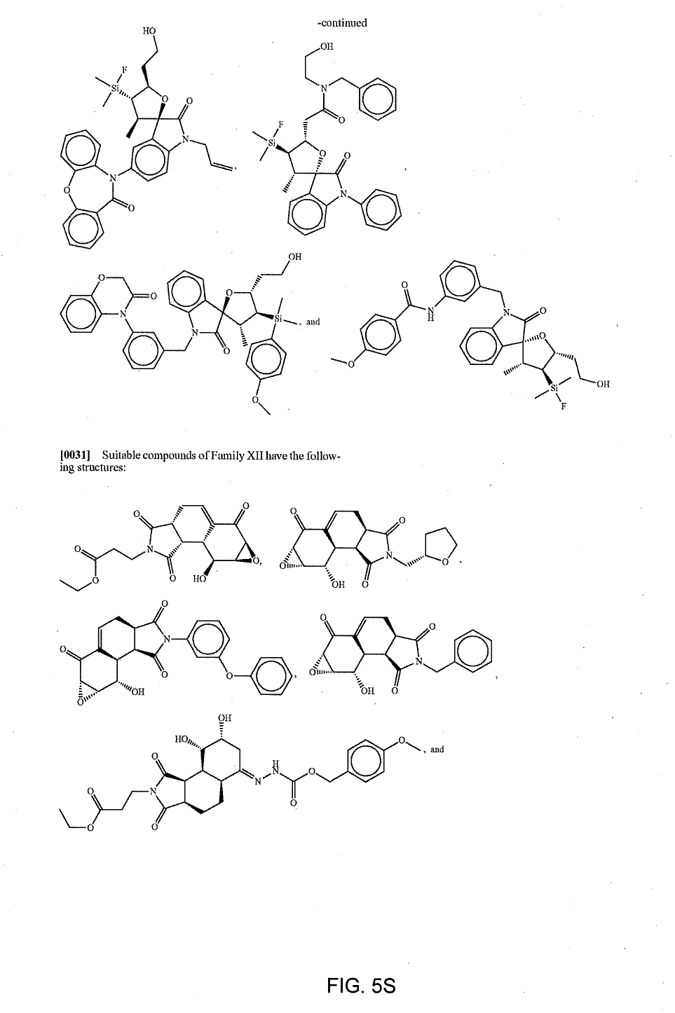

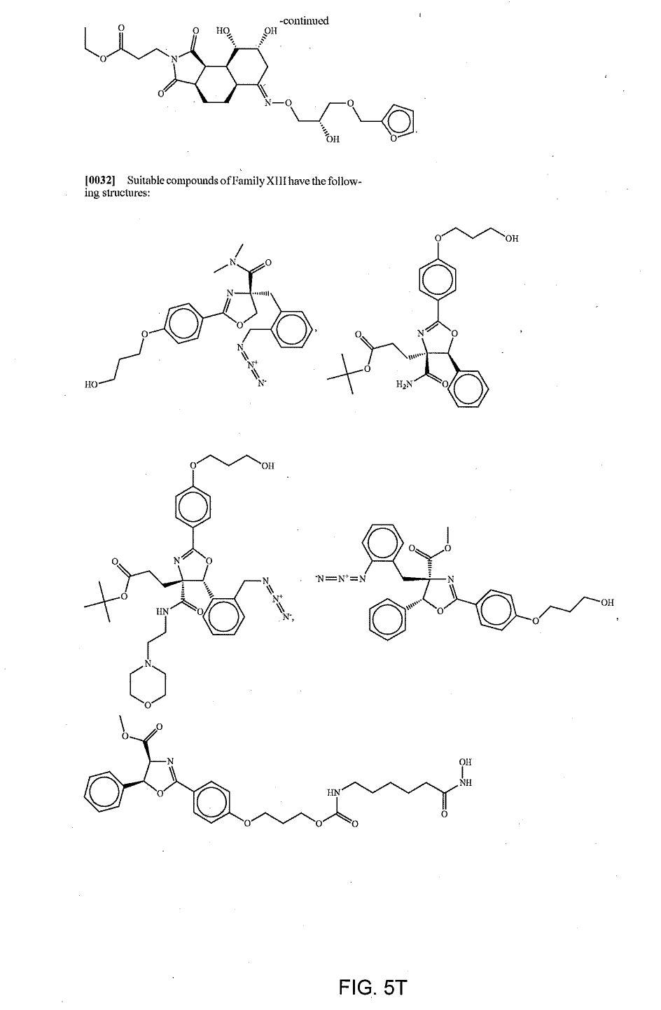

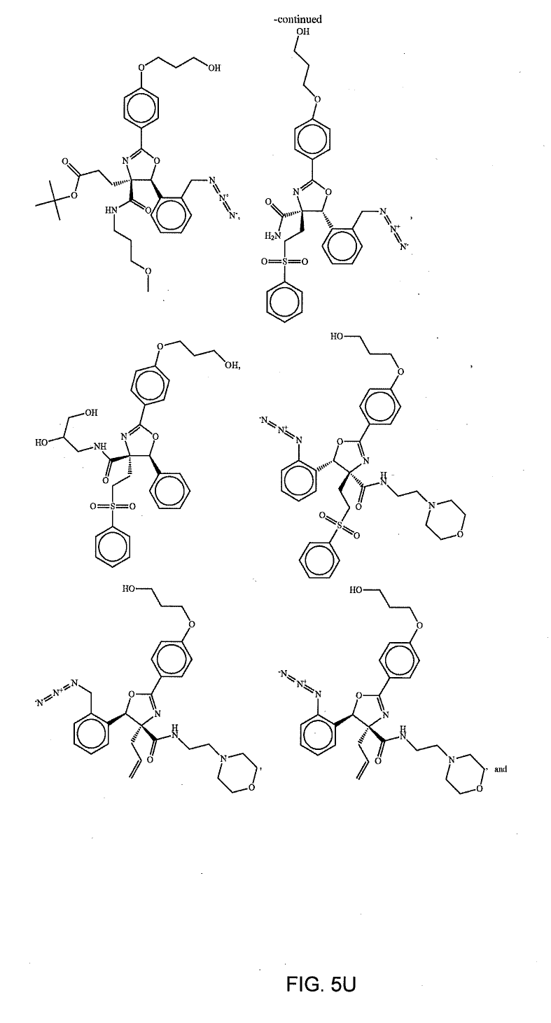

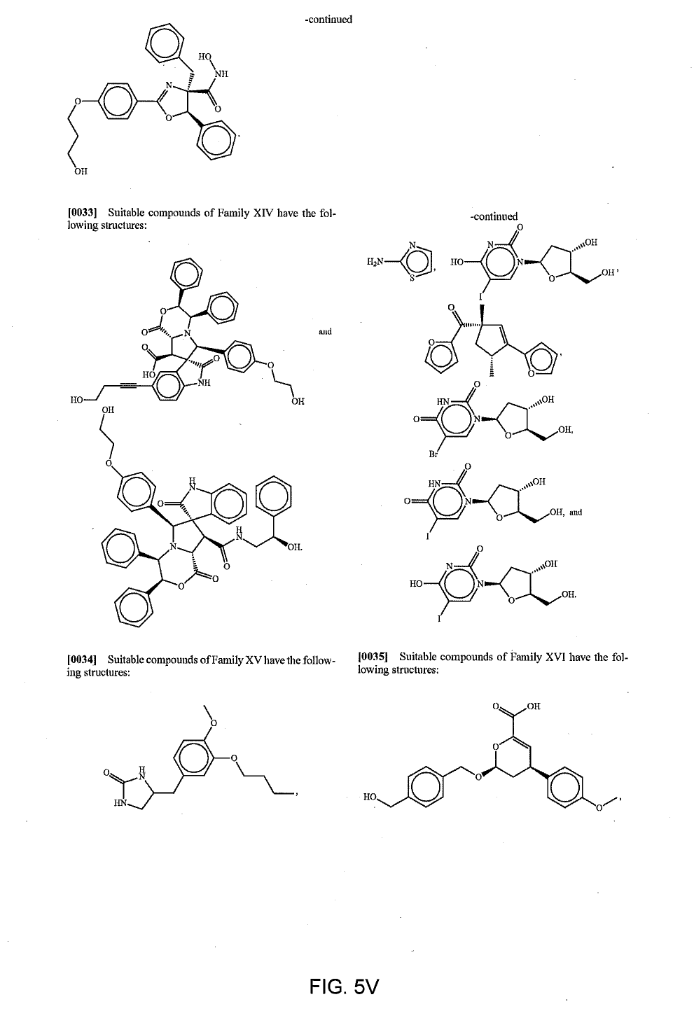

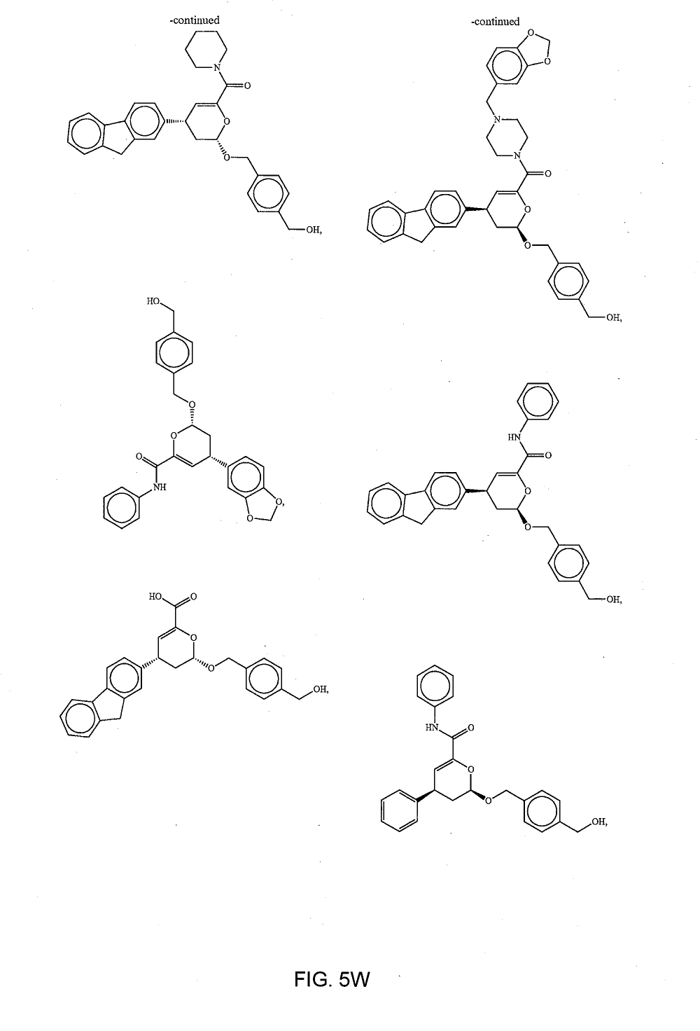

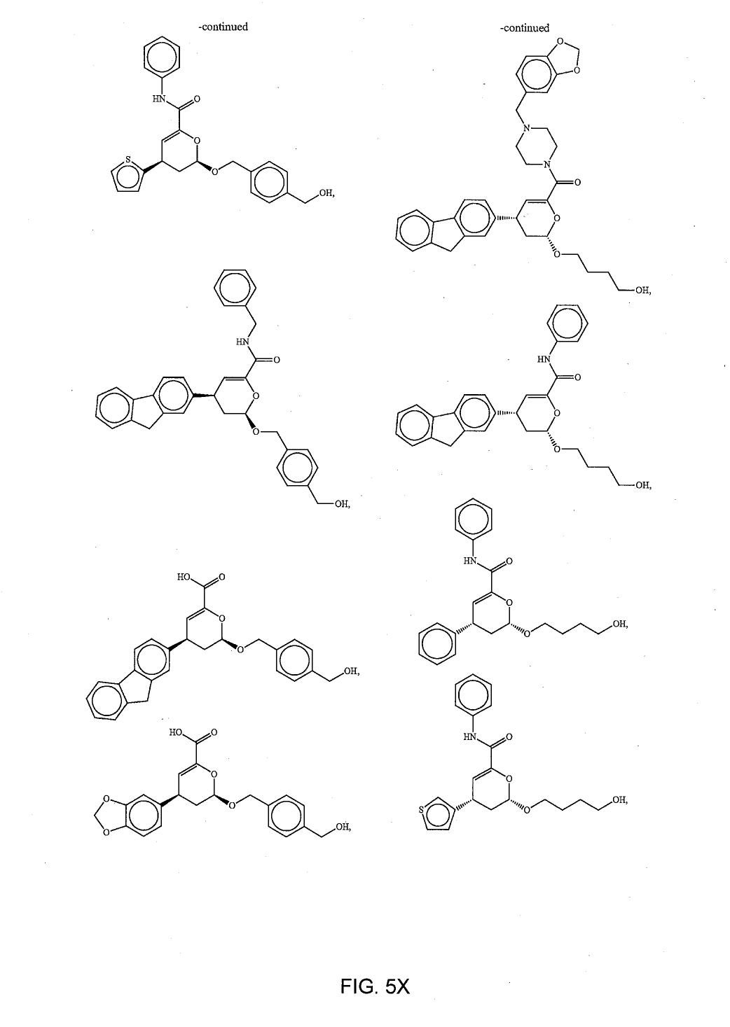

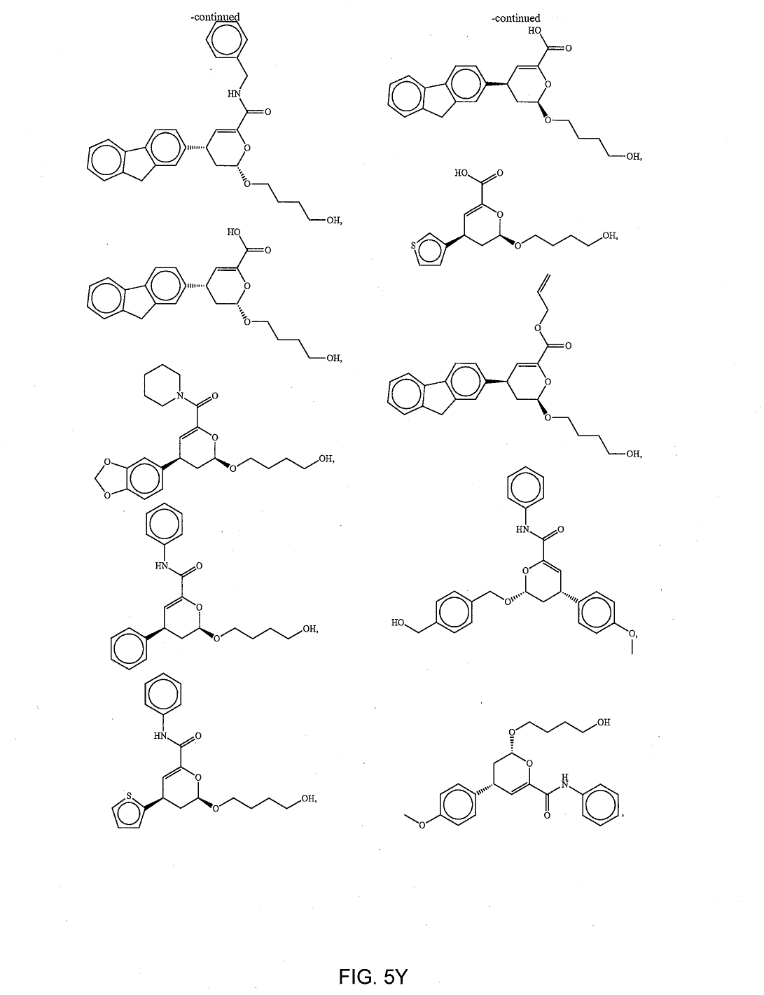

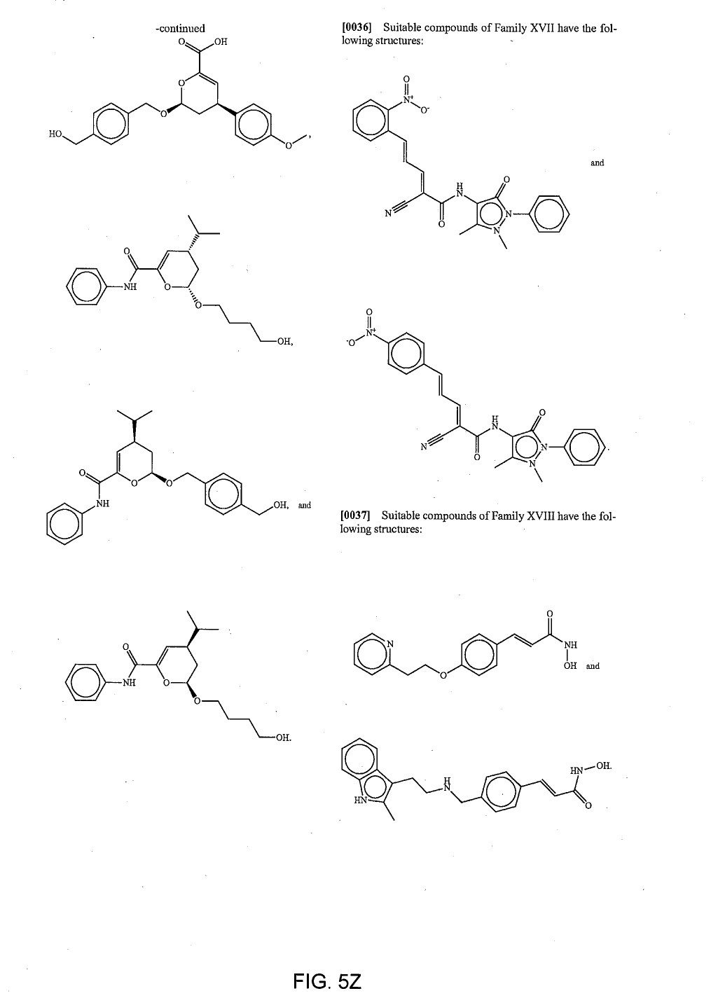

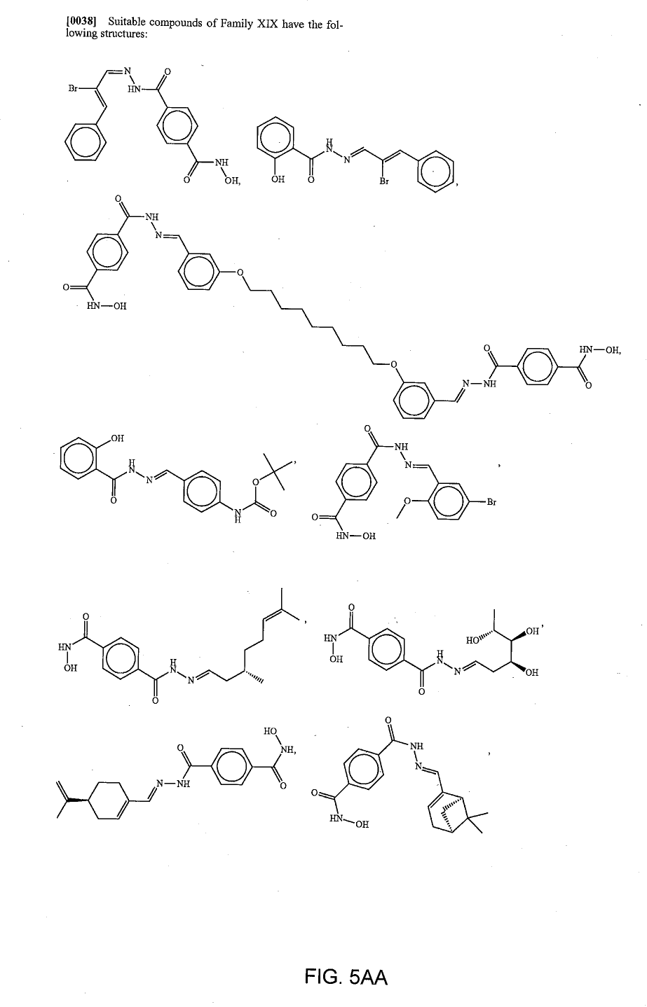

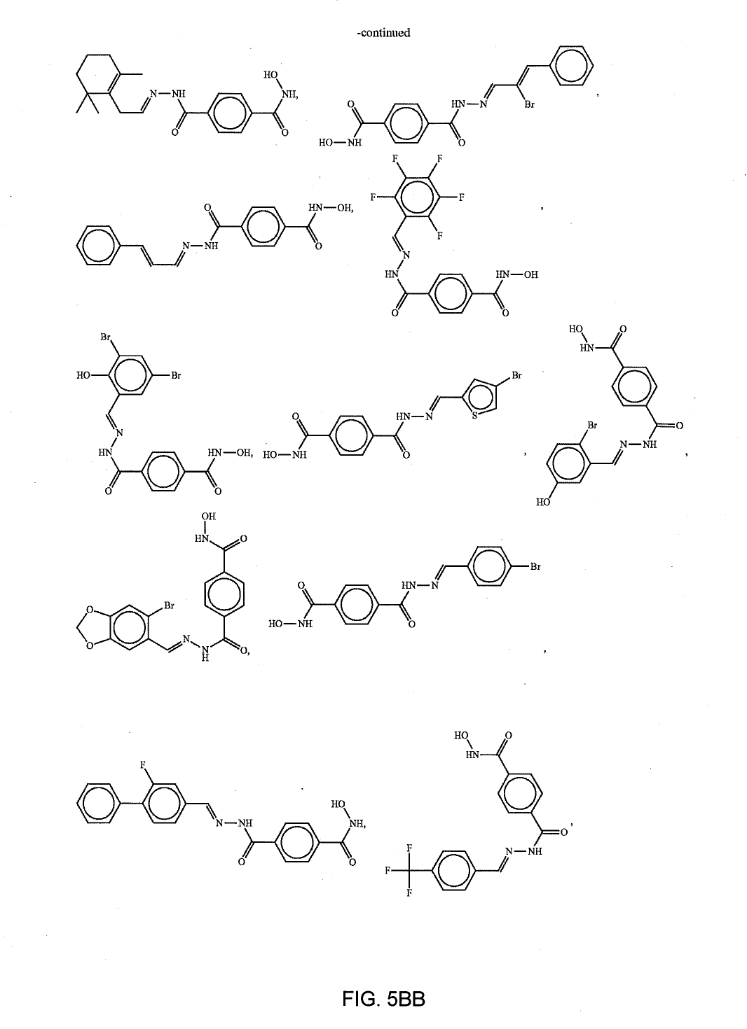

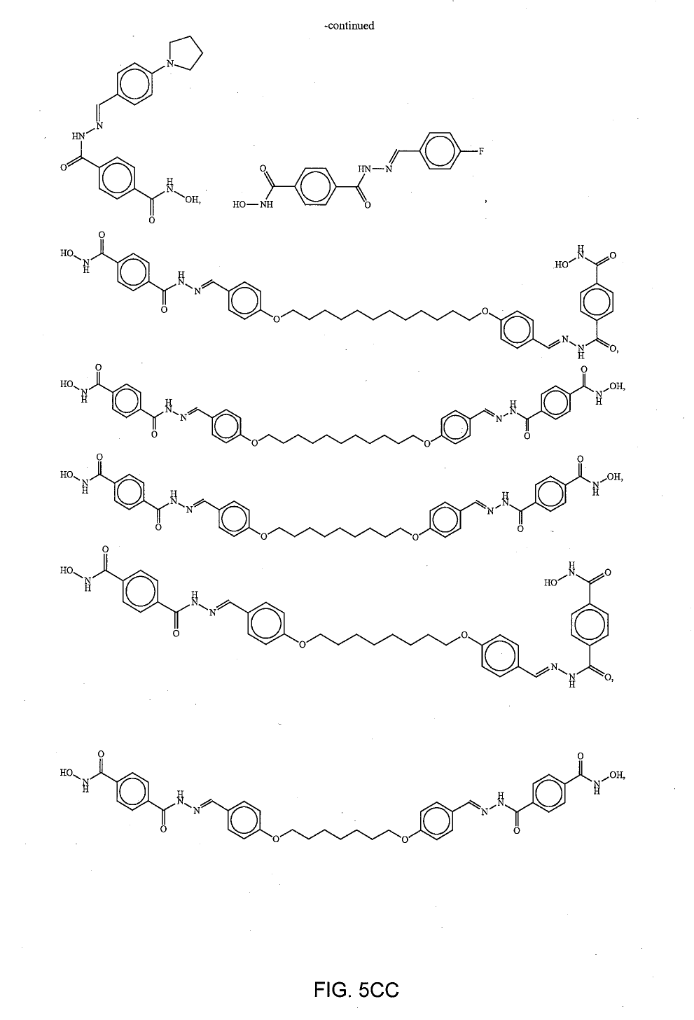

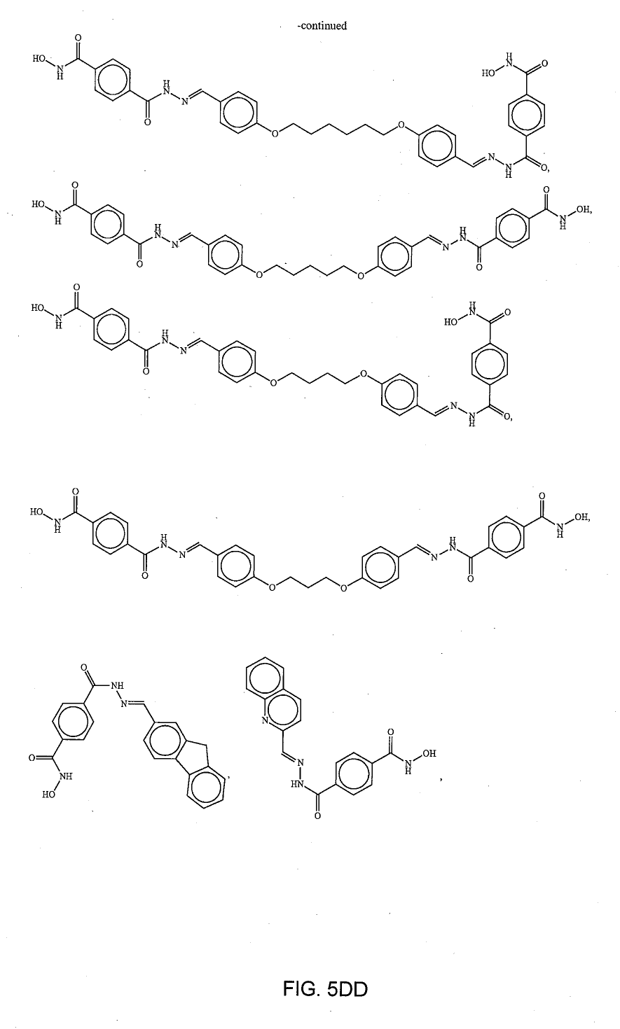

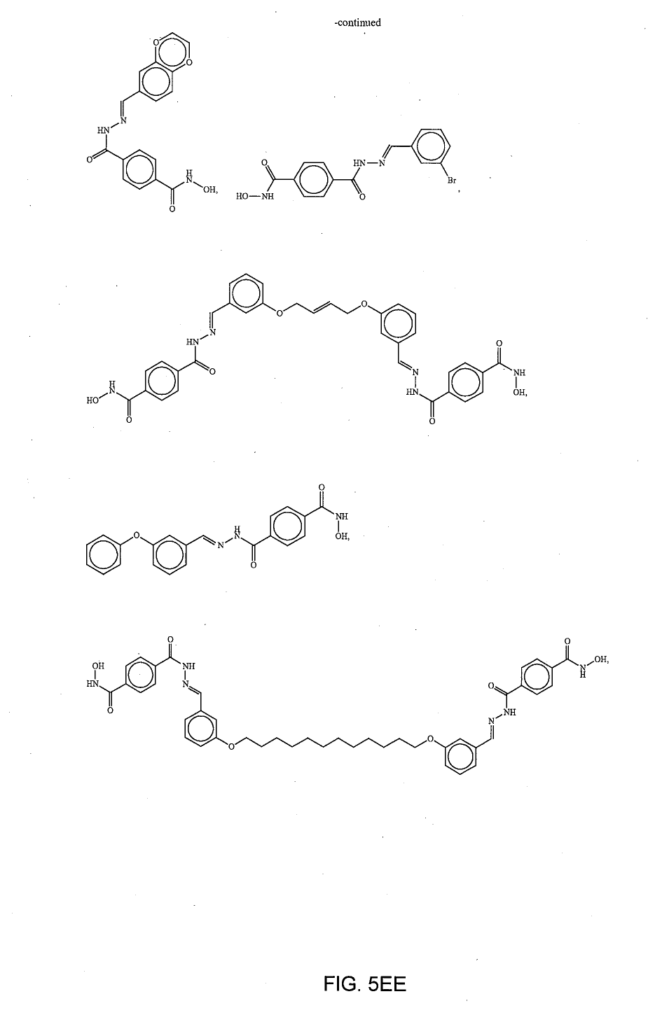

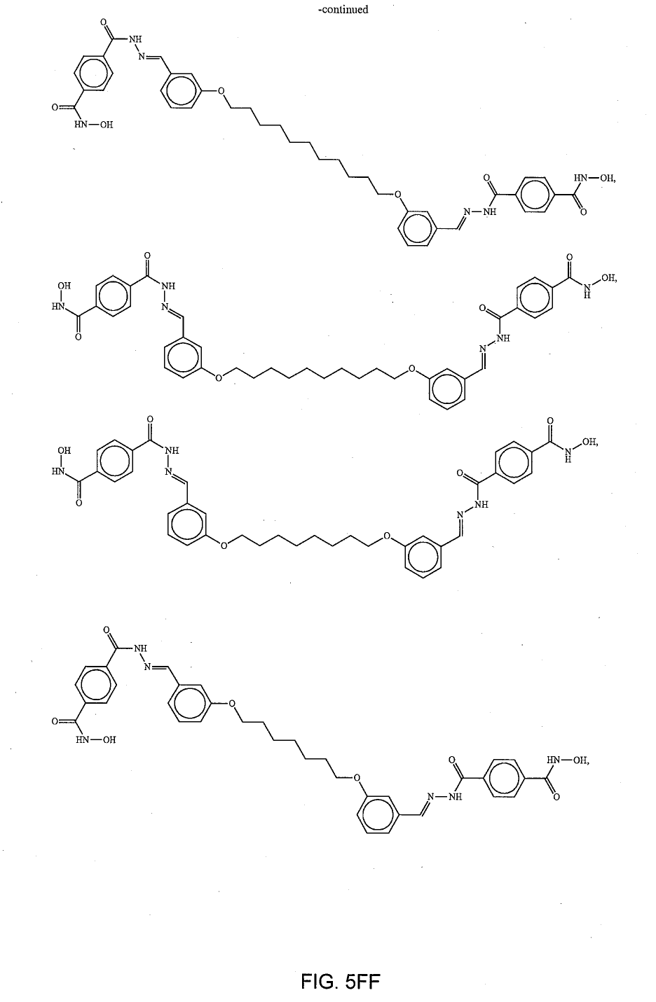

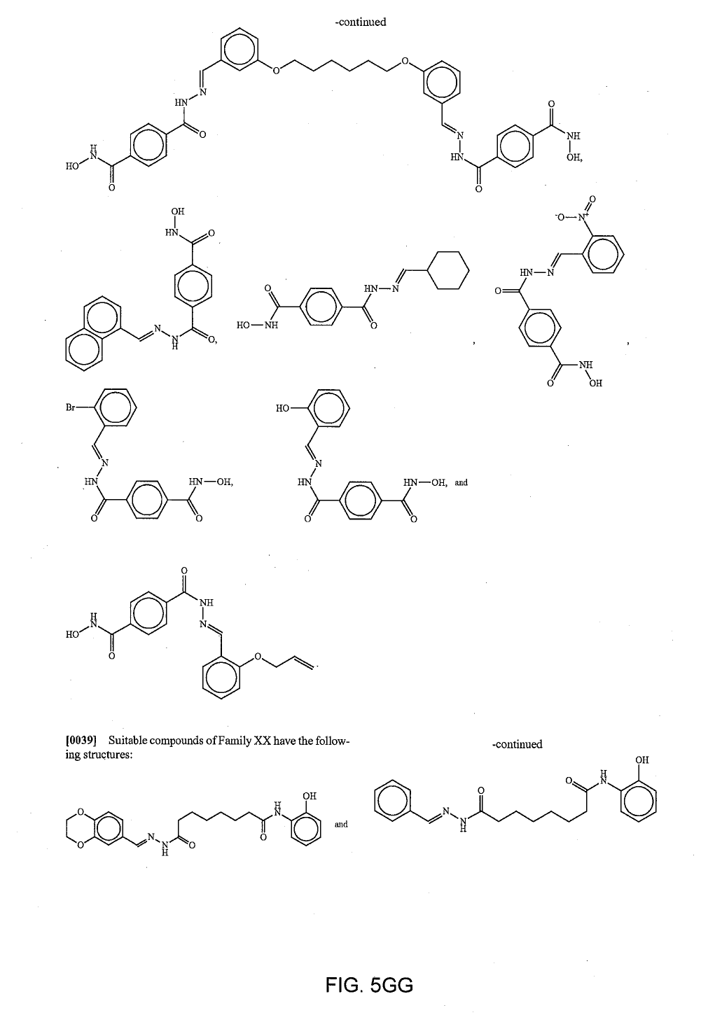

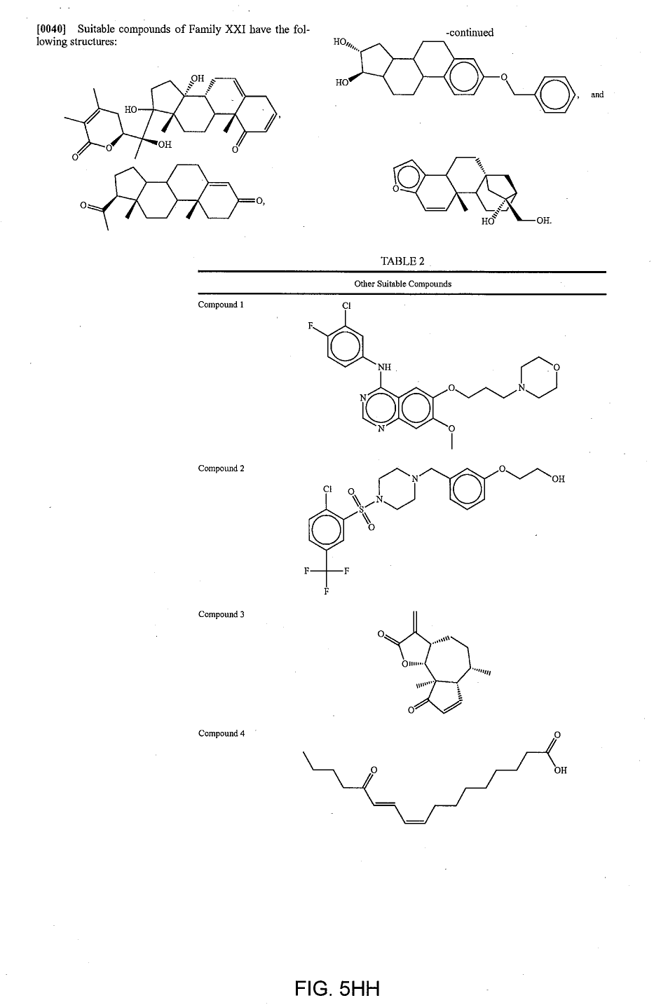

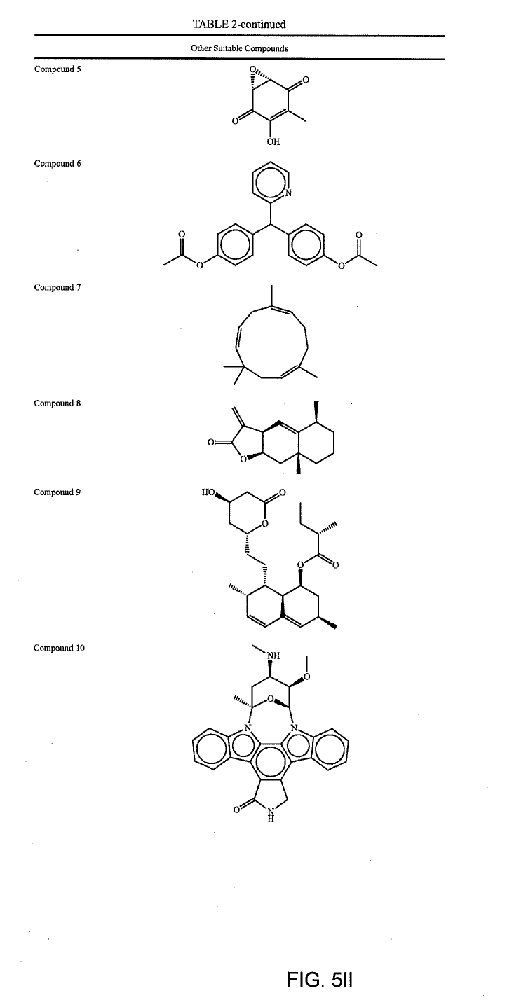

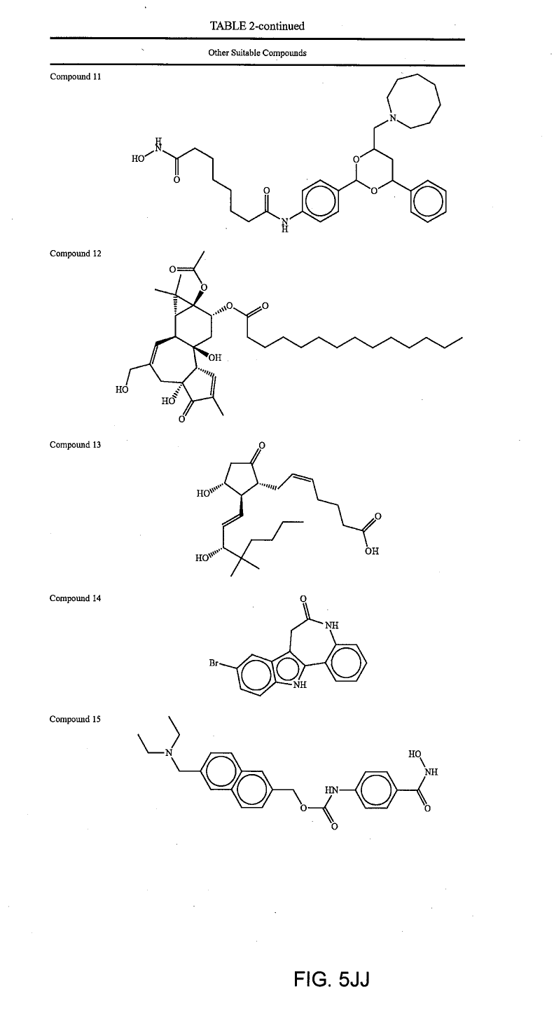

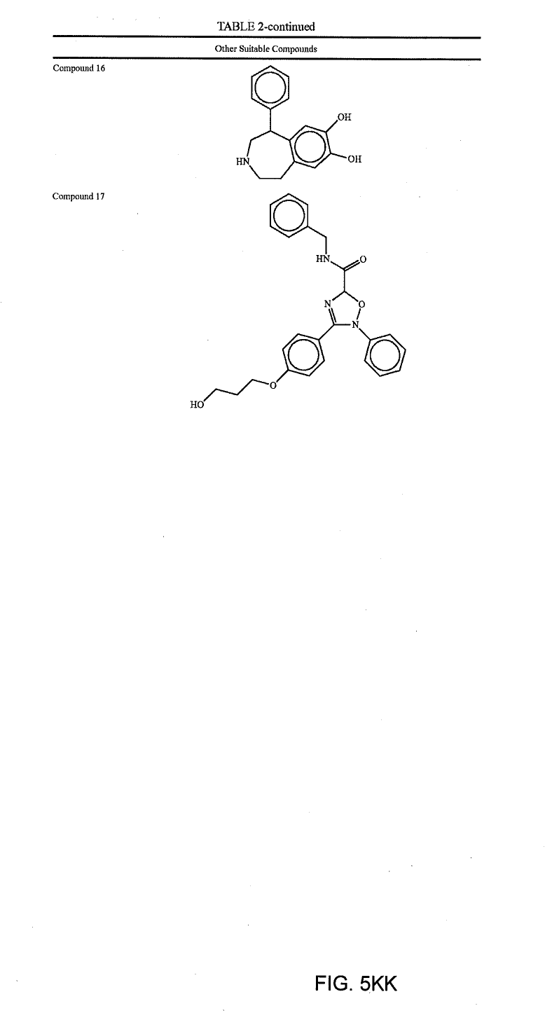

Included in certain contemplated embodiments, but expressly excluded from certain other contemplated embodiments, the choroid plexus inducing agent (CP inducing agent) may be a beta-catenin activator, such as deoxycholic acid (DCA) or any of the compounds set forth herein in FIG. 5, which compounds are disclosed in United States Application Publication Number US2014/0187510.

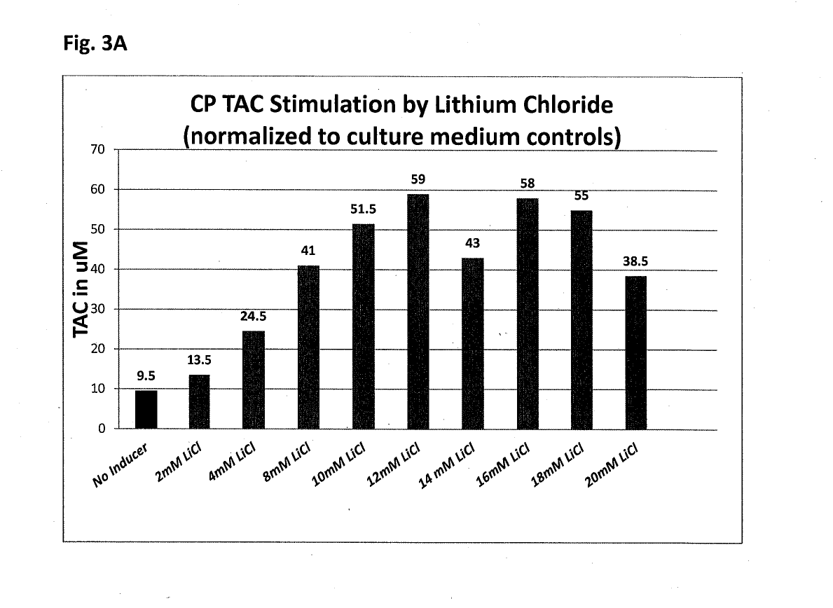

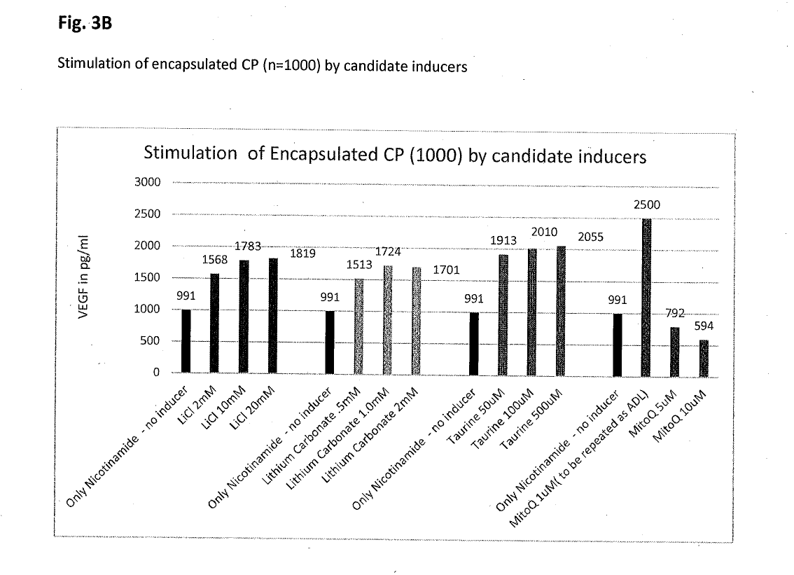

Included in certain contemplated embodiments, but expressly excluded from certain other contemplated embodiments, the choroid plexus inducing agent (CP inducing agent) may be taurine (FIG. 3b). Taurine, or 2-aminoethanesulfonic acid, is a derivative of the amino acid cysteine. While taurine is known to have important roles in the development and function of the nervous system (e.g., Gebara et al., 2015 Stem Cell Res. 14(3):369; Shivaraj et al., 2012 PLoS One 7(8):e42935; Xu et al., 2015 Neurosci Lett. 590:52; El Idrissi et al., 2013 Amino Acids 45(4):735; Jong et al., 2012 Amino Acids 42(6):2223; Schaffer et al., 2009 Can J Physiol Pharmacol. 87(2):91) Gebara et al., 2015; Shivaraj et al., 2012; Xu et al., 2015; El Idrissi et al, 2013) its effects on CP function, including its activity as a CP inducing agent as provided herein, have not been described prior to the present disclosure.

As described herein, CP inducing agents of the present disclosure induce CP cells to alter (e.g., increase or decrease, and in certain preferred embodiments increase) production (including encoding gene expression, biosynthesis, secretion, export, transport, and/or release into the extracellular milieu) of one or more CSF components at a level that differs (i.e., in a statistically significant manner) from the level of production of such CSF component(s) by CP cells without being contacted with the CP inducing agent, which in certain preferred embodiments will be a level that is greater than the level of production when the step of contacting CP cells with a CP inducing agent is omitted. Persons familiar with the art will recognize that CSF components that are produced by CP cells comprise a large number of defined and well characterized peptides, proteins and other biologically active substances (e.g., Redzic et al., 2005 Curr. Topics Dev. Biol. 71:1; see, e.g., FIG. 7) having defined chemical structures that may be detected using established techniques and routine methodologies.

For instance, public database (e.g., GenBank®, National Center for Biotechnology Information, National Institutes of Health, Bethesda, Md.) accession numbers for polynucleotide sequences encoding many CSF components that are peptides or proteins, and for the encoded amino acid sequences of such peptides or proteins, are set forth in FIG. 7 (FIGS. 7A-7J). Accordingly, determination of the production by CP cells of one or more specific CSF components may be achieved by any of a variety of approaches, such as by detection of CSF component-encoding gene expression by a nucleic acid hybridization-based technology, for instance, by polymerase chain reaction (PCR) amplification of CSF component-encoding RNA sequences (e.g., Wang et al., 2009 Nat. Rev. Genet. 10(1):57); and/or by CSF component-encoding RNA or cDNA hybridization to complementary oligonucleotide or polynucleotide sequences present in probe sequence arrays (e.g., GENECHIP® arrays, Affymetrix, Santa Clara, Calif.); and/or by identification of CSF component-encoding transcription products by RNA sequencing (or “RNA-seq”, e.g. Next Generation Sequencing (NGS) using Illumina sequencing by synthesis (SBS) chemistry, Illumina, Inc., San Diego, Calif.) (Bentley et al., 2008 Nature, 456:53); and/or by in situ hybridization (e.g., Yin et al., 1998 Brain Res. 783:347; Swanger et al., 2011 Meths. Mol. Biol. 714:103) or by other nucleic acid detection techniques that are known in the art for determining the presence of specific nucleic acid sequences such as all or portions of any one or more of the RNA sequences or their corresponding DNA sequences encoding any of the CSF components provided herein (e.g., in FIGS. 7 and 8).

Additionally or alternatively, determination of the production by CP cells of one or more specific CSF components (e.g., of FIG. 7 or 8) may be achieved by detection of peptides or proteins or related electrolytes, metabolites or catabolites that comprise such CSF components, for example by specific immunochemical, biochemical, or radiochemical assays or via other detectable indicator moieties, by liquid chromatography and/or mass spectrometry (e.g., Holtta et al., 2015 J. Proteome Res. 14:654; Chiasserini et al. 2014 J. Proteomics 106:191; Naureen et al., 2014 Childs Nerv. Syst. 30:1155; Davidsson et al., 2005 Dis. Markers 21:81; Aluise et al. 2008 Biochim. Biophys. Acta 1782:549; Bonk et al., 2001 Neuroscientist 7:6; Casado et al., 2014 Electrophoresis 35:1181), by functional magnetic resonance imaging (fMRI, e.g., Jasanoff, 2007 Curr. Opin. Neurobiol. 17:593; Bell et al., 2000 Gene Therap. 7:1259), or the like, or by other applicable detection technologies. CSF components are also described, for example, in R. A. Fishman, Cerebrospinal Fluid in Diseases of the Nervous System, W. B. Saunders, Philadelphia, Pa., 1980; Cutler et al., 1982 Ann. Neurol. 11:1; and Hershey et al., 1980 Ann. Neurol. 8:426.

CSF Components

Cerebrospinal fluid (CSF) is produced in the central nervous system (CNS) by choroid plexus epithelial cells, specialized ependymal cells lining the brain ventricles that are noteworthy for their polarization into basolateral and apical membrane domains that possess multiple electrolyte transport channels, and for their constitutive CSF secretory activity. CSF comprises a complex mixture of CSF molecular components that may include without limitation electrolytes, antioxidants, metabolites, mediators and proteins, including variably a number of growth factors, chemotactic factors, chaperone proteins, apolipoproteins, immunoglobulins, hemoglobins, enzymes, defensins, histones, keratins and other cytoskeleton-associated proteins.

CSF composition, including the CSF proteome, has been extensively characterized, and biomarkers associated with a variety of pathologies have been described (e.g., Bora et al., 2012 J. Proteome Res. 11:3143; Whitin et al., 2012 PLoS One 7(11):e49724; Perrin et al., 2013 PLoS One 8(5):364314; Naureen et al., 2013 Fluid Barriers CNS 10:34; Fraisier et al., 2014 PLoS One 9(4):e93637).

Detection of relevant alterations (e.g., statistically significant increases or decreases) in the quantitative representation of one or more CSF components is therefore known to those familiar with the art, for instance, in biological samples containing CSF obtained from human or animal tissues, and also including supernatant fluids or conditioned culture media or the like from cells (e.g., CP cells) or tissues (e.g., CP tissues or tissue fragments) that are capable of CSF production and that have been maintained in vitro under conditions and for a time sufficient to produce CSF or one or more CSF components. Accordingly and in view of the present disclosure, altered (e.g., increased or decreased in a statistically significant manner, relative to an appropriate control) and, for certain preferred CSF components increased, production of one or more CSF components by a CP tissue cell in response to induction by a CP inducing agent, can be determined routinely through the use of existing methodologies.

According to certain embodiments it is contemplated that a choroid plexus inducing agent as provided herein may induce CP tissue cells or in vitro differentiated CP cells to produce altered (e.g., increased or decreased in a statistically significant manner relative to controls), and in certain preferred embodiments increased, levels of one or more CSF components such as the CP products and/or CSF components set forth in FIGS. 7 and/or 8 and including one or more of:

a growth factor that may be IGF-1, IGF-II, FGF-1, bFGF (FGF-2), FGF-9, FGF-12, FGF-18, TGF-β1, TGF-β2, TGF-β3, VEGF, VEGF-A, VEGF-B, VEGF-C/VEGF-2, EGF, growth hormone (GH), BMP-1, BMP-2, BMP-4, BMP-7, BMP-11, BMP-15, GDF-1, GDF-7, GDF-8, GDF-9, GDF-10, GDF-11, nerve growth factor (NGF), PEDF (pigment epithelium derived factor, also known as SerpinF1), glucagon-like peptide-1 (GLP-1), IGF2, BDNF, NT-3, NT-4, GDF-15, GDNF, connective tissue growth factor (CTGF), axotrophin, heparin-binding EGF-like growth factor (HB-EGF), platelet derived growth factor-alpha (PDGF-α), keratinocyte growth factor (KGF), or neurite growth-promoting factor-2/midkine (NEGF2);

a CSF antioxidant that may be ceruloplasmin, superoxide dismutase-1 (SOD-1), superoxide dismutase-2 (SOD-2, Mn-type), superoxide dismutase copper chaperone (CCS), DJ-1/PARK7, catalase, selenoproteins (I, M, N, P, S, T, W, X, 15 kDa), glutathione S-transferase, glutathione S-transferase mu 2 (muscle), glutathione reductase, glutathione peroxidase, hydroxyacyl glutathione hydrolase or thioredoxin;

a chemotactic factor that may be alveolar macrophage-derived chemotactic factor-I (AMCF-I), AMCF-II, stromal cell-derived factor-2, chemokine (CXC motif) ligand 2, chemokines (e.g., CCL8, CCL16, CCL19, CCL21, CCL25, CXCL2, CXCL4, CXCL9, CXCL12, CXCL13, CXCL14), chemokine (CXC motif) receptor-2, chemokine (CXC motif) receptor-4, a chemokine-like factor super family (e.g., CKLF-3, -6, -7), or neurite growth-promoting factor-2/midkine (NEGF2); and/or

a chaperone protein that may be transthyretin, lipocalin-type prostaglandin D synthase/β-trace (L-PGDS), apolipoproteins (e.g., apolipoprotein A, B, C, D, E, H, J, M, N, O, or R), lipocalin-6, lipocalin-7, lipocalin-15, cystatin B, cystatin C, cystatin EM, cystatin 11, a heat shock protein (HSP) family member, or DJ-1 /PARK7.

It will be appreciated that any given CSF component may occur having an amino acid sequence as disclosed herein (e.g., by accession number, or by disclosure in a reference publication incorporated by reference herein, or as known to those familiar with the art, etc.) or may be encoded by a polynucleotide sequence as disclosed herein (e.g., by accession number, or by disclosure in a reference publication incorporated by reference herein, or as known in the art, etc.), and also that any given CSF component may have an amino acid sequence, or may be encoded by a polynucleotide sequence, that is at least 50, 55, 60, 65, 70, 75, 80, 85, 86, 87, 88, 89, 90, 91, 92, 93, 94, 95, 96, 97, 98, or 99 percent identical to, respectively, an amino acid sequence or a polynucleotide sequence as disclosed herein (e.g., by accession number, or by disclosure in a reference publication, etc.) (Stevens et al., 2005 J Mol Recognit 18(2):150). In this regard, CSF components or coding sequences therefore that are less than 100 percent identical to a herein disclosed sequence (e.g., by accession number, etc.) are contemplated as variants, where such variants may result from being the products of accumulated or acquired mutations, allelic variation, posttranslational or posttranscriptional processing, translational or transcriptional error, or the like. Variants are also contemplated where allogeneic or xenogeneic tissues are the sources of CP cells, for instance, where an allogeneic or xenogeneic homologue of a herein disclosed CSF component may be at least 50, 55, 60, 65, 70, 75, 80, 85, 86, 87, 88, 89, 90, 91, 92, 93, 94, 95, 96, 97, 98, or 99 percent identical to, respectively, an amino acid sequence or a polynucleotide sequence as disclosed herein (e.g., by accession number, etc.).

When comparing polypeptide (amino acid) or polynucleotide sequences, two sequences are said to be “identical” if the sequence of amino acids or nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign™ program in the Lasergene™ suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345; Hein J., 1990 Unified Approach to Alignment and Phylogenes, pp. 626; Methods in Enzymology vol. 183, Academic Press, Inc., San Diego, Calif.; Higgins, D. G. and Sharp, P. M., 1989 CABIOS 5:151; Myers, E. W. and Muller W.,1988 CABIOS 4:11; Robinson, E. D., 1971 Comb. Theor 11:105; Santou, N. Nes, 1987 M., Mol. Biol. Evol. 4:406; Sneath, P. H. A. and Sokal, R. R.,1973 Numerical Taxonomy—the Principles and Practice of Numerical Taxonomy, Freeman Press, San Francisco, Calif.; Wilbur, W. J. and Lipman, D. J.,1983 Proc. Natl. Acad., Sci. USA 80:726.

Alternatively, optimal alignment of sequences for comparison may be conducted by the local identity algorithm of Smith and Waterman, 1981 Add. APL. Math 2:482, by the identity alignment algorithm of Needleman and Wunsch, 1970 J. Mol. Biol. 48:443, by the search for similarity methods of Pearson and Lipman, 1988 Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Preferred examples of algorithms that are suitable for determining percent sequence identity and sequence similarity are the BLAST and BLAST 2.0 algorithms, which are described in Altschul et al.,1977 Nucl. Acids Res. 25:3389, and Altschul et al., 1990 J. Mol. Biol. 215:403, respectively. BLAST and BLAST 2.0 can be used, for example with the parameters described herein, to determine percent sequence identity among two or more polypeptides or polynucleotides. Software for performing BLAST analyses is publicly available through the National Center for Biotechnology Information.

In one illustrative example, cumulative scores can be calculated using, for nucleotide sequences, the parameters M (reward score for a pair of matching residues; always >0) and N (penalty score for mismatching residues; always <0). Extensions of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff, 1989 Proc. Natl. Acad. Sci. USA 89:10915) alignments, (B) of 50, expectation (E) of 10, M=5, N=−4 and a comparison of both strands.

Alternatively, the sequences obtained from RNA sequence analysis (e.g., RNA-seq, described above and in the Examples) are aligned to a reference genome. For example, RNA sequence reads for each sample can be mapped to a reference genome (e.g., Ensembl Sscrofa10.2, and Database for Annotation, Visualization, and Integrated Discovery (DAVID), Sam borski et al., Transcriptome changes in the porcine endometrium during the preattachment phase, 2013 Biol Reprod. 2013 Dec. 12; 89(6):134); Dennis et al., DAVID: Database for Annotation, Visualization, and Integrated Discovery, 2003 Genome Biol. 4(5):P3; Huang et al., DAVID Bioinformatics Resources: expanded annotation database and novel algorithms to better extract biology from large gene lists. 2007 Nucleic Acids Res. 2007 July; 35(Web Server issue):W169-75; Huang et al., Systematic and integrative analysis of large gene lists using DAVID bioinformatics resources, 2009 Nat Protoc. 2009; 4(1):44-57) using Tophat (v2.0.13) software to align RNA-seq reads to a reference genome, CuffLinks software to assemble reads that have been mapped by Tophat into potential transcripts to generate an assembled transcriptome, and CuffDiff software to accept the reads assembled from two or more different biological conditions and analyze them for differential expression of genes and transcripts under the different conditions (e.g., induced versus control conditions). (See, e.g., Ghosh et al., Analysis of RNA-Seq Data Using TopHat and Cufflinks. 2016 Methods Mol. Biol. 2016; 1374:339-61). For library normalization, various methods, such as classic-fpkm, geometric, quartile or other methods can be applied (See, e.g., http website: //cole-trapnell-lab.github.io/cufflinks/cuffdiff/#library-normalization-methods) combined with various cross-replicate dispersion estimation methods (e.g., pooled, per-condition, bline, or poisson methods, See, e.g., http website: //cole-trapnell-lab.github.io/cufflinks/cuffdiff/#library-normalization-methods).

By way of a non-limiting illustrative example, Differentially Expressed Genes (DEGs) may be identified using ‘gene_exp.diff’ output from the Cuffdiff software program. To detect DEGs between controls and ‘induced’ samples, two filtering processes can be applied. First, using a Cuffdiff status code, genes that only have “OK” status in each sample are obtained. Status code ‘OK’ indicates that each condition contains sufficient sequence reads in a locus for a reliable calculation of expression level and that the test is successful to calculate gene expression level in that sample. For the second filtering, a two-fold change in expression level is calculated and only genes displaying more than two-fold changes between the samples being compared (control vs. induced) are selected. For ontology analysis, the selected gene list is applied to DAVID software (Huang et al. 2009 Nat Protoc. 2009; 4(1):44-57; Huang et al. 2007 Nucleic Acids Res. 2007 35(Web Server issue):W169-75; Dennis et al., 2003 Genome Biol. 4(5):P3) to obtain a comprehensive set of functional annotations. Categories such as gene-disease association, homologue match, gene ontology, or pathway categories, etc. can be selected. DAVID then generates a functional annotation chart which lists annotation terms and their associated genes.

In certain embodiments, the “percentage of sequence identity” is determined by comparing two optimally aligned sequences over a window of comparison of at least 20 positions, wherein the portion of the polypeptide or polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acids residues or nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that may encode a particular CSF component polypeptide as described herein. Some of these polynucleotides bear minimal sequence identity to the nucleotide sequence of the original polynucleotide sequence that encodes the CSF component polypeptide having an amino acid sequence that is disclosed herein. Nonetheless, polynucleotides that vary due to differences in codon usage are expressly contemplated by the present disclosure. In certain embodiments, sequences that have been codon-optimized for mammalian expression are specifically contemplated.

CP Cell Sources

The presently disclosed embodiments relate to improved biocompatible, non-immunogenic, semi-permeable alginate capsules containing therapeutic xenogeneic and/or allogeneic CP cells for administration into the CNS. In certain embodiments CP tissue fragments may be prepared and CP cell-containing capsules selected as described elsewhere herein, by modifying previous teachings directed to CP xenotransplantation. General methodologies for the preparation and use of such capsules are described, for example, in U.S. Pat. No. 6,322,804, U.S. Pat. No. 5,834,001, U.S. Pat. No. 6,083,523, US2007/134224, U.S. Pat. No. 5,869,463, US2004/213768, US2009/0214660, and US2009/0047325. Implantation in the brain of xenogeneic choroid plexus tissue fragments within biocompatible capsules for the treatment of CNS diseases is described, for example, in US2007/134224, and in US2004/213768, US2005/0265977, U.S. Pat. No. 6,083,523, and US2009/0047325 and related patent application publications. US2009/0047325 describes an exemplary preparation of neonatal CP cells for xenotransplantation.

With respect to the biological sources of CP tissues and/or CP cells, however, the present embodiments are not intended to be so limited, such that there are also presently contemplated embodiments in which mammalian choroid plexus tissue may be obtained from a mammal that is xenogeneic relative to the subject being treated with the herein described selected and induced biocompatible, non-immunogenic, semi-permeable alginate capsules containing therapeutic xenogeneic CP cells. CP tissue may thus be obtained from porcine, ovine, bovine, caprine, non-human primate, or other mammalian sources. In certain other embodiments the CP cells may be obtained from a biological source that is allogeneic to the subject undergoing treatment, e.g., the source may be tissue from a non-genetically identical individual of the same species as the subject.

In certain illustrative exemplary embodiments, allogeneic or xenogeneic pluripotent cells that are capable of differentiation into CP cells may be cultured in vitro under conditions and for a time sufficient to obtain a plurality of in vitro differentiated CP cells. Conditions for in vitro generation of human CP cells from human embryonic stem cells (ESC), and of mouse CP cells from murine ESC, are described, by way of example, in Watanabe et al., 2012 J. Neurosci. 32(45):15934 and Sternberg et al., 2014 Regen Med 9(1):53. Pluripotent cells for use in these and related embodiments may comprise embryonic cells such as embryonic stem cells, embryonic stem cell-derived clonal embryonic progenitor cell lines, neural crest progenitors and/or may also comprise one or more of non-embryonic cells, such as umbilical cord cells, placental cells, dental pulp cells, adult tissue stem cells and/or mesenchymal stem cells from somatic tissues, for which methods of preparation will be known to those skilled in the relevant art (e.g., Loeffler et al., 2002 Cells Tissues Organs 171(1):8-26).

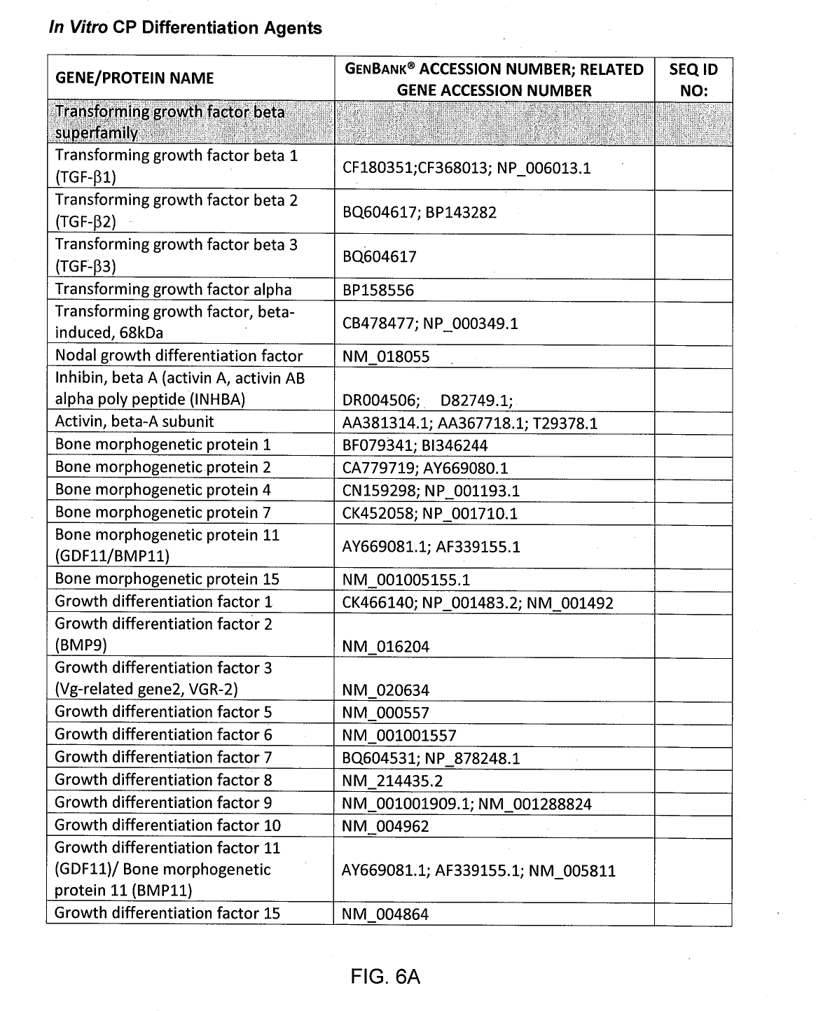

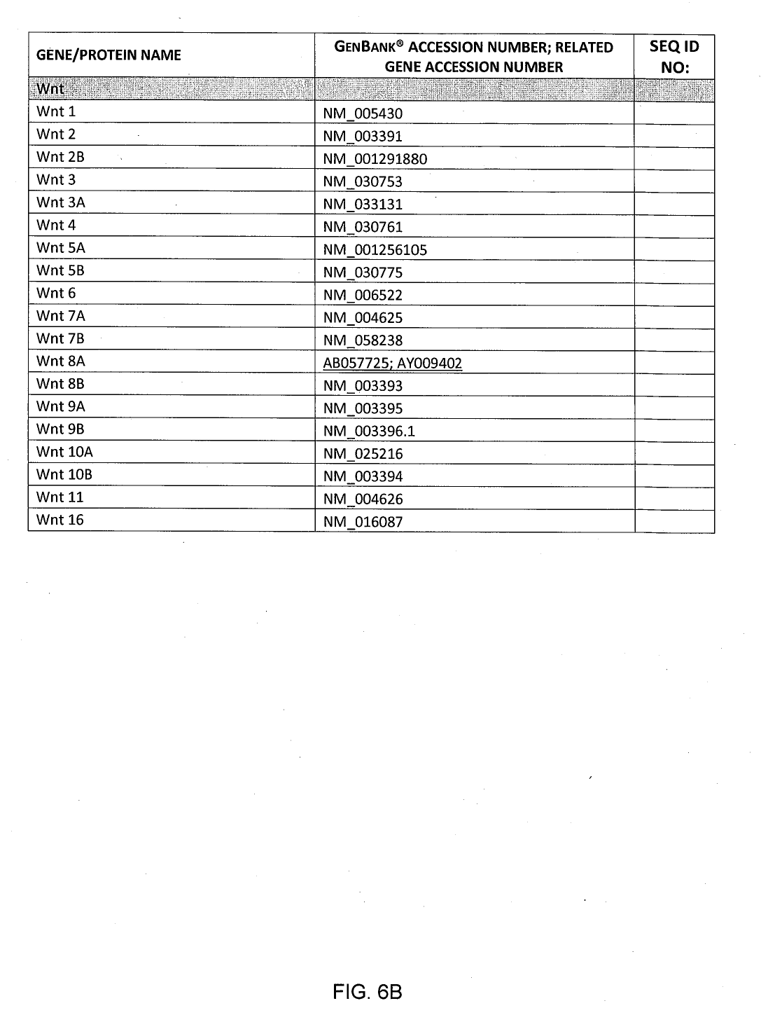

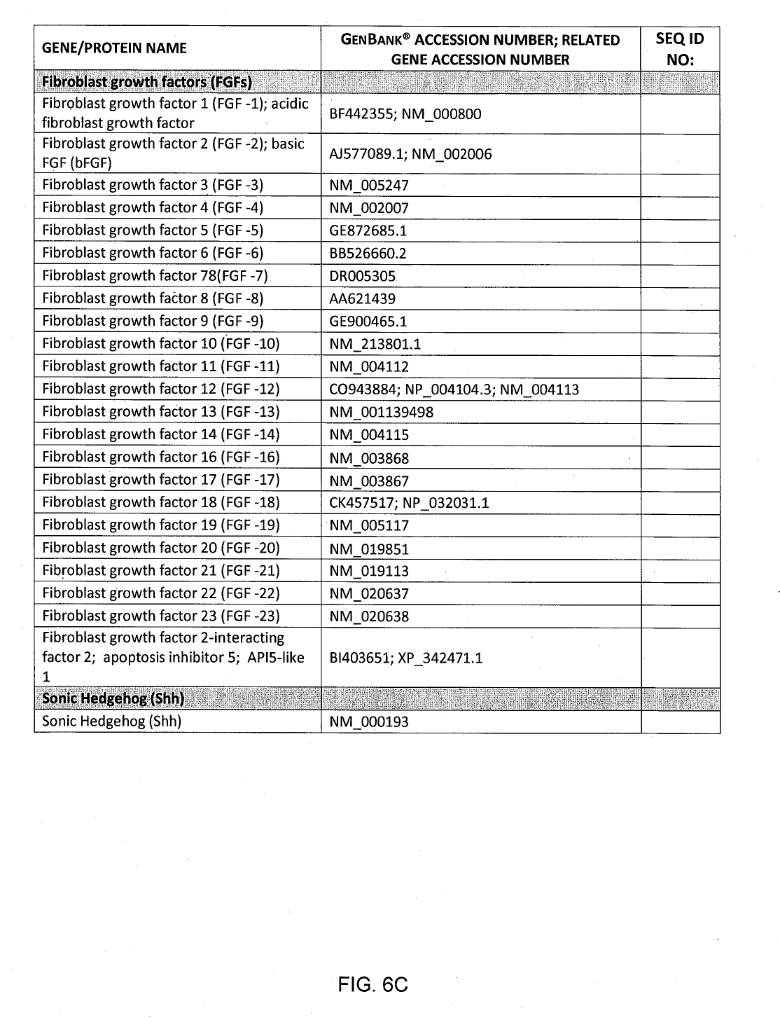

In these and related embodiments, pluripotent cells may be cultured in a culture medium that comprises one or more in vitro CP differentiation agents such as any of the in vitro CP differentiation agents disclosed in FIG. 6 (FIG. 6A-6C). For example, pluripotent cells may be cultured in a culture medium that comprises one or more of a bone morphogenic protein (BMP) or a BMP signaling pathway agonist, a transforming growth factor-beta (TGF-β) superfamily member or a TGF-β signaling pathway agonist, a mammalian growth and differentiation factor (GDF) or a GDF signaling pathway agonist, VEGF, a Wnt protein ligand or a Wnt signaling pathway agonist, sonic hedgehog (Shh), a Shh signaling pathway agonist (e.g., a synthetic small molecule agonist such as purmorphamine and/or SAG, see Stanton et al. 2009 Mol. BioSyst 6:44), and a fibroblast growth factor (FGF) or an FGF signaling pathway agonist, under conditions and for a time sufficient to obtain said plurality of in vitro differentiated choroid plexus (CP) cells (see, e.g., Watanabe et al., 2012 Jl. Neurosci. 32(45):15934; Sternberg et al. 2014, Regen Med, 9(1):53; see also, e.g., Ward et al. 2015 Neuroscience S0306-4522(15)00415; Liddelow, 2015 Front. Neurosci. 9 (32):1); Huang et al. 2009 Development 340(2):430); Schober et al., 2001 J Comp Neurol 439(1):32).

For example, a Wnt signaling pathway agonist may comprise one or more of WAY-316606 (SFRP inhibitor, 5-(phenylsulfonyl)-N-4-piperidinyl-2-(trifluoromethyl)benzene sulfonamide hydrochloride, Bodine et al., 2009 Bone 44:1063), IQ1 (PP2A activator, Miyabayashi et al., 2007 Proc. Nat. Acad. Sci. USA 104:5668), QS11 (ARFGAP1 activator, Zhang et al., 2007 Proc. Nat. Acad. Sci. USA 104:7444), (hetero)arylpyrimidine or 2-amino-4-[3,4-(methylenedioxy) benzyl-amino]-6-(3-methoxyphenyl) pyrimidine, Norrin (e.g., Ohlmann et al., 2012 Prog. Retin Eye Res. 31:243; Rey et al., 2010 Dev. Dyn 239:102; erratum 2010 Dev. Dyn. 239:1034; GenBank Acc. No. NM_000266), R-spondin-1 (e.g., Peng et al., 013 Cell Rep. 3:1885; GenBank Acc. No. NM_001038633; NM_001242910.1), R-spondin-2 (e.g., GenBank Acc. No. NM_178565; NM_178565.4; NM_001282863.1), R-spondin-3 (e.g., GenBank Acc. No. NM_032784), and R-spondin-4 (e.g., GenBank Acc. No. NM_001029871.3). A Wnt signaling pathway agonist may also, in certain embodiments, comprise any suitable lithium salt, i.e., a lithium compound that comprises cationic lithium and that can be contacted with cells with no or minimal toxicity, for example, lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or any other lithium salt as may be known to those skilled in the relevant art.

Certain other preferred embodiments contemplate encapsulated choroid plexus tissue fragments that are prepared from tissue that is xenogeneic relative to the subject undergoing treatment. For example, for the treatment of humans it is envisioned that xenogeneic encapsulated CP cells may be obtained from a non-human source, preferably a non-human mammalian source. In certain such embodiments the non-human mammalian source of CP tissue containing CP cells that are encapsulated in the herein described semi-permeable biocompatible (e.g., alginate) capsules may be porcine tissue. Certain further embodiments relate to neonatal porcine CP tissue as the source of CP cells to be encapsulated for use in the present methods, where “neonatal” may be understood to include tissue that is obtained at any time from immediately after birth until up to three months of age.

According to certain embodiments of the present disclosure there are also provided surprising advantages that derive from the use of fetal or neonatal CP tissue (such as fetal or neonatal porcine CP tissue) that is substantially free of human pathogens, and in particular that may be substantially free of human-tropic transmissible porcine endogenous retroviruses (PERVs). It is to be understood that “substantially free” refers to a situation where conventional means for detecting human pathogens or conventional means for detecting human-tropic transmissible PERVs fail to detect such pathogens or PERVs in a statistically significant manner and with a degree of confidence of at least 95%, 96%, 97%, 98% or 99%.

In this regard, PERVs represent a serious health and safety risk accompanying the use of porcine tissues and cells for xenotransplantation into humans, despite many characteristics that make porcine tissues and cells well-suited for such transplants. In particular, PERVs that may be present in porcine donor cells to be used for transplantation are capable of infecting human cells (Fishman, 1998 Ann. NY Acad. Sci. 862:52; Elliott et al., U.S. Pat. No. 8,088,969; Park et al., 2008 J. Microbiol. Biotechnol. 18:1735; Hector et al. 2007 Xenotransplant. 14:222). By contrast, on Auckland Island, New Zealand, there has been identified a population of domesticated pigs (Sus scrofa domesticus) that has been shown to be pathogen-free by a set of defined criteria, and that had missing from their genomes a full length PERV-C locus that had previously been associated with the ability of PERV to infect human cells (Garkavenko et al., 2008 Cell Transplant. 17:1381; Hector et al. 2007 Xenotransplant. 14:222). The pathogen-free animals included a subset of pigs that lacked a PERV-C env gene which is capable of recombination with a PERV-A env gene (Id.).

Accordingly, it is contemplated that in the practice of certain embodiments of the present disclosure, the xenogeneic tissue source of CP cells, which are present in semi-permeable biocompatible capsules that are selected, administered and induced as described herein, will comprise fetal or neonatal porcine CP tissue that is substantially free of human-tropic PERVs. In certain further embodiments the CP tissue is obtained from an animal that lacks a PERV-C env gene which is capable of recombination with a PERV-A env gene or that has been genetically engineered to lack any or all PERV genes using an established gene editing technique such as Clustered Regularly-Interspaced Short Palindromic Repeats (CRISPR)-Cas9 editing (e.g., Jinek et al., 2012 Science 337:816; Doudna et al., 2014 Science 346:1258096).

CP Preparation and Encapsulation

In general, the materials, methods and techniques that may be employed to practice certain of the presently disclosed embodiments may be achieved by incorporating the improvements described herein into adaptations of the teachings relating to choroid plexus tissue and cell preparations, to semi-permeable biocompatible capsules such as alginate capsules and the like, and/or to CNS administration including brain implantation of capsules, that may be found in one or more of the publications of Elliott and colleagues (e.g., US 2009/0047325; U.S. Pat. No. 8,129,186), Vasconcellos et al. (e.g., US 2009/0214660), Dionne et al. (e.g., U.S. Pat. No. 6,322,804; U.S. Pat. No. 6,083,523), Major et al. (e.g., U.S. Pat. No. 5,753,491), Monuki et al. (e.g., U.S. Pat. No. 8,748,176; see also Watanabe et al., 2012 Jl. Neurosci. 32(45):15934) and/or in US 2007/0134224 (Harlow et al.), U.S. Pat. No. 4,892,538 (Aebischer et al.), and/or US 2012/0003190 (Yamoah et al.), all of which are incorporated by reference but which individually or in any combination fail to teach or suggest the improvements according to the presently disclosed combinations.

According to certain preferred embodiments as disclosed herein for the first time, it has been surprisingly discovered that particular advantages may be obtained by including a step of selecting one or more semi-permeable biocompatible capsules (e.g., alginate capsules) in which are encapsulated CP tissue fragments and/or in vitro differentiated CP cells according to the presently recited methods of treating.

Specifically, and in a manner that could not have been predicted prior to the present disclosure, the dimensions of the capsules to be administered to a CNS site, and the number of CP cells contained in each capsule, contribute to the CSF component production level by encapsulated cells on a per cell basis. Counterintuitively and according to non-limiting theory, increased CSF component production per cell, including CSF component per cell following induction with a CP inducing agent, was not simply and directly proportional to the number of cells present in each capsule, but was instead found to be achieved using capsules selected to have diameters of from about 400 μm to about 800 μm and typically having internal volumes of less than about one microliter, and that contained about 200 to about 10,000 cells per capsule, where “about” may be understood to represent quantitative variation that may be more or less than the recited amount by less than 50%, more preferably less than 40%, more preferably less than 30%, and more preferably less than 20%, 15%, 10% or 5%.

In certain preferred embodiments semi-permeable biocompatible capsules are thus selected that each contain at least about 200, 400, 600, 800, 1000, 2000, 3000, 4000, 5000, 7500 or 9000 and not more than about 10,000 CP cells. In certain preferred embodiments capsules are selected that each contain at least about 400, 600, 800, 1000, 1500, 2000, 2500, 3000, 3500, 4000, 4500, 5000, 5500, 6000, 6500, 7000, or 7500 and not more than about 8000 cells.

In certain preferred embodiments semi-permeable biocompatible (e.g., alginate) capsules may be prepared that have diameters of from about 400 μm to about 800 μm, from about 500 μm to about 700 μm, from about 450 μm to about 750 μm, or from about 400 μm to about 700 μm, and that typically each have an internal volume of less than about one microliter.

Selection may be accomplished by any of a variety of techniques with which the skilled person will be familiar. For example, semi-permeable biocompatible capsules prepared as described herein and according to established methodologies set forth in the cited reference documents may be visualized under a microscope and manually selected according to calibrated occupancy by cells of the included volume (e.g., empirically established consistent capsule occupancy, and/or by using colorimetric or fluorescent markers such as vital stains or DNA-binding dyes, etc.) using a micromanipulator, a microneedle, a microcapillary pipette, a patch-clamp device, or the like. Alternatively, automated equipment such as a large particle flow sorter (e.g., COPAS™ FlowPilot™ platform, Union Biometrica Inc., Holliston, Mass., USA), particle size analyzer, digital image analyzer, flow cytometer or the like may be used to process preparations of semi-permeable biocompatible capsules containing encapsulated CP cells.

In preferred embodiments, the present semi-permeable biocompatible capsules in which are “encapsulated” CP tissue fragments and/or in vitro differentiated CP cells include those capsules that, upon visual microscopic inspection, exhibit substantially no cells or portions of cells that are detectable on exterior surfaces of the capsules and substantially no cells or portions of cells protruding from a capsule interior to the capsule surface.

According to non-limiting theory, it is believed that selection according to the presently described criteria of capsule diameter, number of encapsulated CP cells, and substantial freedom of capsular exterior surfaces from cells or portions of cells, including from cells or portions of cells protruding from the capsule interior to the exterior capsule surface, advantageously results in capsules that elicit little or no detectable tissue rejection (e.g., immune rejection) or inflammatory reaction (e.g., foreign body response) subsequent to administration or implantation of the capsules to a central nervous system (CNS) site in a subject, such as implantation in brain tissue of a mammalian subject known to have or suspected of having a nervous system disease. Additionally, encapsulated CP cells that are administered to a subject by implantation in a CNS site in vivo according to the present methods exhibit surprising and unexpected longevity, and may remain viable in the semi-permeable biocompatible (e.g., alginate) capsules for greater than 2, 3, 4, 5, 6, 7, 8, 9, 10, 11, 12, 13, 14, 15, 16, 18, 20, 24 or more months post-implantation, substantially without elicitation of localized immunological or chronic inflammatory reactions such as immune rejection of the CP cell-containing capsules, host extracellular matrix deposition on the capsules, or a foreign body response to the capsules. Accordingly and in certain embodiments, the capsules do not elicit chronic inflammation at the CNS site following implantation in the course of administration, and/or administration of an immunosuppressant agent (e.g., Craft et al., 2005 Exp. Opin. Ther. Targets 9:887; Jha et al., 2014 Recent Pat. Inflamm. Allergy Drug Discov. 8:118; Bellavance et al., 2014 Front. Immunol. 5:136; Lossinsky et al., 2004 Histol. Histopath. 19:535, and references cited therein) to the subject is not required to ameliorate immunological rejection of the capsules at the CNS site.

Moreover, the small enclosed volumes of the semi-permeable biocompatible (e.g., alginate) capsules that are selected as disclosed herein permit efficiency and economy in the preparation and delivery of encapsulated CP cell implants, and, by virtue of the herein described step of contacting with a CP inducing agent, further provide the ability to deliver a potent CSF source to brain tissue whilst occupying minimal tissue space at the implantation site, thereby minimizing the amount of tissue disruption that accompanies the step of administering.

Nervous System Disorders

Persons skilled in the relevant art will be familiar with any number of diagnostic, surgical and/or other clinical criteria that may indicate the clinical appropriateness of, and/or to which can be adapted, administration of the encapsulated CP cell compositions described herein. See, e.g., Sontheimer, Diseases of the Nervous System, 2015 Academic Press/Elsevier, Waltham, Mass.; “Neurologic Disorders” in The Merck Manual of Diagnosis and Therapy 19^{th} Ed. (R. S. Porter, Ed., 2011, Merck, Inc., NJ); “Neurological Diagnostic Tests and Procedures” at the website of the National Institute of Neurological Disorders and Stroke, National Institutes of Health, Bethesda, Md., www.ninds.nih.gov/disorders/misc/diagnostic_tests.htm; Neurology in Clinical Practice-Vol. II, 4^{th} Edition, Bradley et al., (Eds), 2004 Butterworth Heinemann/Elsevier, Philadelphia, Pa.; Non-Neoplastic Diseases of the Central Nervous System (Atlas of Nontumor Pathology-First Series Fascicle), D. N. Lewis et al., (eds.), 2010 Amer. Registry of Pathology, Annapolis Junction, Md.; Bradley's Neurology in Clinical Practice (6^{th} Ed.), R. B. Daroff et al. (eds.), 2012 Saunders/Elsevier, Waltham, Mass. Criteria for diagnosis and clinical monitoring of patients having or suspected of having disorders or diseases of the nervous system are thus well known to those skilled in the relevant art.

Accordingly, it is contemplated that the herein described compositions and methods may find beneficial uses in a wide range of nervous system diseases for which the presence or risk for having in a subject will be apparent to the skilled clinician. Non-limiting examples of nervous system diseases to be treated according to the teachings found herein therefore include, e.g., Parkinson's disease, multiple system atrophy-Parkinson type, multiple system atrophy-cerebellar type, progressive supranuclear palsy, dementia with Lewy bodies, essential tremor, drug-induced Parkinsonism, Alzheimer's disease, Huntington's disease, amyotrophic lateral sclerosis (ALS), prion disease, motor neuron disease, spinocerebellar ataxia, spinal muscular atrophy, static nervous diseases such as stroke, CNS trauma, seizure disorders including epilepsy; progressive neurodegenerative diseases including those associated with aging and dementia, such as Alzheimer's disease, Parkinson's disease, etc.; diseases of motor neurons and neuromuscular junctions; Huntington's disease; multiple sclerosis; CNS tumors, especially brain tumors, including neuroblastoma, glioma, astrocytoma; infectious diseases of the nervous system including meningitis, botulism, tetanus, neurosyphilis, poliomyelitis, rabies, HIV/AIDS, prion diseases, Naegleria fowleri (amoebic brain infection); neurocysticerosis; neuropsychiatric diseases including depression, mood disorders; obsessive-compulsive disorder, schizophrenia; diseases associated with or characterized by one or more of neuronal death, glutamate toxicity, protein aggregates and/or deposits (e.g., amyloid plaque formation), mitochondrial dysfunction including reactive oxygen species (ROS) production levels in excess of those found in normal, healthy control subjects; brain derived neurotrophic factor-related disorders, and other nervous system diseases.

In certain embodiments there is thus provided a method of treating a subject known to have or suspected of having a nervous system disease, wherein the nervous system disease is a neurodegenerative disease that is characterized by death of neurons. For example, these and related embodiments contemplate a method of treating a subject known to have or suspected of having a nervous system disease wherein the nervous system disease may be at least one of Parkinson's disease, Alzheimer's disease, Huntington's disease, amyotrophic lateral sclerosis (ALS, otherwise known as Motor neurone disease), progressive bulbar palsy, progressive muscular atrophy, dementia with Lewy bodies, multiple system atrophy, spinocerebellar ataxia type 1 (SCA 1), or an age-related neurodegenerative disorder. The encompassed embodiments are not intended to be so limited, however, such that methods are also contemplated of treating other neurodegenerative diseases that are characterized by death of neurons.

In certain embodiments there is provided a method of treating a subject known to have or suspected of having a nervous system disease, wherein the nervous system disease is characterized by a decrease in a level of at least one nerve cell function, relative to the level of the nerve cell function in a control subject known to be free of the nervous system disease. For example, these and related embodiments contemplate a method of treating a subject known to have or suspected of having a nervous system disease wherein the nervous system disease may be at least one of Parkinson's disease (in which there is a decrease in the level of function of dopaminergic neurons), Alzheimer's disease (in which there is a decrease in the level of function of noradrenergic neurons, see, e.g., Adori et al. 2015, Acta Neuropathol 129(4):541), Huntington's disease (in which there is a decrease in the level of function of medium spiny GABA neurons, (MSN)), amyotrophic lateral sclerosis (ALS, in which there is a decrease in the level of function of motor neurons), and depression (in which there is a decrease in the level of function of serotoninergic neurons).

In certain embodiments there is provided a method of treating a subject known to have or suspected of having a nervous system disease, wherein the nervous system disease is characterized by an increase in a level of at least one nerve cell function, relative to the level of said nerve cell function in a control subject known to be free of the nervous system disease. For example, these and related embodiments contemplate a method of treating a subject known to have or suspected of having a nervous system disease wherein the nervous system disease may be at least one of psychosis, schizophrenia (in which there is an increase in the level of nerve cells that may be manifest as hyperactive dopamine signaling); epileptic seizures (in which there is an increase in the level of nerve cells that may be manifest as glutamatergic excitotoxicity), ischemic stroke (in which there is an increase in the level of nerve cells that may be manifest as glutamatergic excitotoxicity), and insomnia associated with restless leg syndrome (in which there is an increase in the level of nerve cells that may be manifest as overactive glutamatergic activity).

In certain embodiments there is provided a method of treating a subject known to have or suspected of having a nervous system disease, wherein the nervous system disease is characterized by presence in the subject of cerebrospinal fluid (CSF) that comprises an altered level of one or more cerebrospinal fluid (CSF) components, relative to the level of said CSF component or components in a control subject known to be free of the nervous system disease. Representative CSF components are set forth in FIG. 7. For example, these and related embodiments contemplate a method of treating a subject known to have or suspected of having a nervous system disease wherein the nervous system disease may be at least one of Alzheimer's disease and diabetes mellitus.

In certain embodiments there is provided a method of treating a subject known to have or suspected of having a nervous system disease, wherein the nervous system disease is characterized by presence in the subject of an altered level of at least one choroid plexus function, relative to the level of said choroid plexus function in a control subject known to be free of the nervous system disease. For example, these and related embodiments contemplate a method of treating a subject known to have or suspected of having a nervous system disease wherein the nervous system disease may be Sturge-Weber syndrome, or Klippel-Trenaunay-Weber syndrome, or any of a number of other clinically identifiable congenital nervous system diseases having recognized diagnostic signs and symptoms.

In certain embodiments there is provided a method of treating a subject known to have or suspected of having a nervous system disease, wherein the nervous system disease in the subject is a secondary effect of increased (e.g., in a statistically significant manner) amyloid deposit in the endothelium and smooth muscle cells in the nervous system of the subject, relative to the level of said deposit in a control subject known to be free of the nervous system disease (e.g., Ghiso et al., 2001 J. Alzheimer's Dis. 3:65). For example, these and related embodiments contemplate a method of treating a subject known to have or suspected of having a nervous system disease wherein the nervous system disease may be cerebral amyloid angiopathy, hereditary cerebral hemorrhage with amyloidosis-Icelandic type (HCHWA-I), cerebral hemorrhage with amyloidosis-Dutch type (HCHWA-D), meningocerebrovascular and oculoleptomeningeal amyloidosis, gelsolin-related spinal and cerebral amyloid angiopathy, familial amyloidosis-Finnish type (FAF), vascular variant prion cerebral amyloidosis, familial British dementia (FBD), otherwise known as familial cerebral amyloid angiopathy-British type or cerebrovascular amyloidosis-British type, familial Danish dementia, also known as heredopathia ophthalmo-oto-encephalica, familial transthyretin (TTR) amyloidosis, or PrP cerebral amyloid angiopathy (PrP-CAA) (Ghiso et al. 2001 J Alzheimer's Dis 3:65).

Method of Treating

Preferred embodiments contemplate a method of treating a subject that is a human or non-human mammal known to have or suspected of having a nervous system disease. Mammals thus may include humans, and also may include domesticated animals such as laboratory animals, livestock and household pets (e.g., rodents, cats, dogs, rabbits and other lagomorphs, swine, cattle, sheep, goats, horses, other ungulates, etc.), and also non-domesticated animals such as wildlife and the like.

A “therapeutically effective amount” refers to that amount of a composition or preparation according to the present disclosure which, when administered to a mammal, preferably a human, is sufficient to effect treatment of a nervous system disease or condition in the mammal, preferably a human. The amount of such a composition or preparation, such as one or more selected semi-permeable biocompatible capsules in which are encapsulated choroid plexus (CP) cells as described herein and/or a choroid plexus inducing agent as provided herein, which constitutes a “therapeutically effective amount” will vary depending on the composition or preparation, the nervous system disease or condition and its severity, and the age of the mammal to be treated, but can be determined routinely by one of ordinary skill in the art having regard to such person's own knowledge and to this disclosure.

“Treating” or “treatment” refers to therapy to heal, relieve symptoms of and/or correct underlying defects contributing to or causes of the nervous system disease, disorder or condition of interest in a mammal, preferably a human, having the disease or disorder of interest (e.g., a neurodegenerative disease), and includes inhibiting (e.g, impairing, reducing or preventing, such as decreasing in a statistically significant manner) or repairing (e.g., replacing, supplementing or substituting for) a defective molecular, cellular, and/or tissue component that contributes to the nervous system disease, disorder or condition and/or a deleterious process that contributes to the nervous system disease, disorder or condition, to a substantial and statistically significant degree of inhibition or repair (although not necessarily complete), e.g., at least 5%, 10%, 20%, 30%, 40%, 50%, 60%, 70%, 80%, 85%, 90%, 95% or greater inhibition or repair relative to appropriate untreated controls; and also includes partially or completely relieving the signs or symptoms resulting from the disease, disorder or condition, e.g., reducing inflammatory lesions associated with disease, restoring one or more normal neuronal and/or glial cell structures and/or functions, etc.

General methodologies for preparation and implantation of biocompatible, semi-permeable alginate capsules containing CP cells into CNS sites are described, for example, in U.S. Pat. No. 6,322,804, U.S. Pat. No. 5,834,001, U.S. Pat. No. 6,083,523, US2007/134224, U.S. Pat. No. 5,869,463, US2004/213768, US2009/0047325, and related publications including the references cited therein, and may be modified according to the teachings herein. Surgical procedures known in the art therefore are contemplated for adaptation, in view of the present disclosure, to certain embodiments in which the step of administering the capsules to the CNS injection site comprises delivering the capsules through a catheter, which may, for example, comprise an external catheter, an obdurator, a plunger, or a delivery catheter. In certain further embodiments, delivering comprises controllably positioning the catheter with a stereotactic apparatus, which may in certain still further embodiments comprise a deep brain stimulator (DBS) microdriver or a similar apparatus as may be modified for use in the present methods.

In certain embodiments of the above described methods, administering the capsules to the CNS injection site (or in certain other embodiments, to a PNS injection site) comprises delivering the capsules through a catheter. In certain further embodiments, administering comprises delivering by controllably positioning the catheter with a stereotactic apparatus. In certain still further embodiments, the stereotactic apparatus may comprise by way of exemplary illustration and not limitation, a deep brain stimulator (DBS) microdriver, a “frameless” stereotactic head frame, a skull-mounted aiming device, a Leksell frame, a Cosman-Roberts-Wells frame, or another similar modified stereotactic apparatus or the like, or any equivalent, for example, any of the devices described in Bot et al., 2015 Stereotact. Funct. Neurosurg. 93:316; Sharma et al., 2014 Neurol. India 62:503; Larson et al., 2012 Neurosurg. 70(1 Suppl Operative):95; Kelman et al., 2010 Stereotact. Funct. Neurosurg. 88:288; Starr et al., 2010 J. Neurosurg. 112:479; Starr et al., 2009 Neurosurg. Clin. N. Am. 20:193. In certain embodiments the catheter comprises an external catheter, an obdurator, a plunger, and a delivery catheter.

In these and related embodiments, administration of one or a plurality of the herein described capsules may comprise delivery to a desired anatomical location referred to herein in certain preferred embodiments as a CNS injection site (or a PNS injection site) as provided herein. Administration may comprise delivery, for example, via a dual catheter delivery system that may be specific for the particular medical indication being treated and/or for the target injection site for delivery. An exemplary dual catheter delivery system may comprise an external guide catheter system and an internal capsule delivery system. The external guide catheter system may be blunt-ended and designed to reduce tissue damage upon insertion into CNS tissue or PNS tissue, and is also designed to create a space in the appropriate target (recipient) tissue to be occupied by one or a plurality of delivered capsules following capsule delivery at the injection site when the catheter is partially or fully withdrawn from the tissue. Capsules are loaded into the internal catheter using a plunger system, and the so-loaded internal catheter system is then inserted into the external guide catheter system for guided delivery using, for instance and in certain preferred embodiments, a targeting/guiding/aiming device such as a stereotactic apparatus.

For example, to ensure controlled capsule delivery that is specific to the indication being treated, the entire capsule delivery device at the targeted site in CNS or PNS tissue may be slowly retracted in precise increments in a stepwise manner using a microdrive system, thereby creating a space in the CNS (or PNS) tissue that is hereby referred to as an injection site and which can accommodate one or more delivered capsules that are controllably released by the capsule delivery device. Between each incremental retraction of the delivery device, the plunger of the internal catheter may be depressed to slowly deliver one or more capsules into the space at the spatial location (e.g., the injection site) created by the withdrawal of the external catheter. This process of incrementally retracting the entire capsule delivery device followed by depressing the plunger of the internal catheter to deliver one or a plurality of capsules to the injection site (e.g., a CNS injection site or a PNS injection site) may be repeated until all or substantially all capsules for which delivery to the injection site is desired have been delivered to the targeted site. The configuration of capsule arrangement within the internal catheter and/or in the space at the spatial location (e.g., the injection site) created by withdrawal of the capsule delivery device may in some embodiments be provided as a layered configuration of capsules in the injection site in which each layer comprises 2, 3, 4, 5, 6, 7, 8, 9, 10, 11, 12, 13, 14, 1, 5, 16, 17, 18, 19, 20 or more capsules, and/or may in certain other embodiments be provided as a unidimensional columnar configuration of stacked single capsules in an injection site that is proportioned so as to have a diameter that can accommodate only one capsule per layer. The preferred configuration of the injection site and of the capsular arrangement within such injection sites may be varied as a function of the particular medical indication for which treatment is desired and/or as a function of the specific anatomical location of a desired injection site and/or as may be appropriate for the number of capsules to be delivered at each site. The configuration of capsules in the injection site may also be controlled, for instance, by varying the distance over which the entire capsule delivery device is retracted, relative to the distance by which the plunger of the internal catheter is depressed to controllably release a desired number of capsules into the injection site.

For instance, semi-permeable biocompatible capsules prepared as described herein have been administered into the putamen of a patient with Parkinson's disease by release from a catheter into a CNS injection site formed as a delivery tract at the end of a single catheter delivery system, to obtain at the injection site a column of stacked capsules. Snow et al., June 2015, Safety and clinical effects of NTCELL® [immunoprotected (alginate-encapsulated) porcine choroid plexus cells for xenotransplantation] in patients with Parkinson's disease (PD): 26 weeks follow-up. Poster session presented at 19^{th} International Congress of Parkinson's Disease and Movement Disorders, San Diego, Calif., USA.

As noted elsewhere herein, certain contemplated embodiments relate to a method that comprises administering the herein described biocompatible capsules containing CP cells to one or more peripheral nervous system (PNS) sites such as a PNS injection site. Such administration to a PNS injection site may be performed by employing methodologies that have been developed in the relevant art for treatment of the PNS. Administration to a PNS injection site can be achieved by introduction of CP-containing capsules via guided catheters or other suitable instrumentation, corresponding, for instance, to the instrumentation/ apparatus as described herein for CNS sites. For administration to a PNS injection site, persons familiar with the art will recognize any of a number of anatomical locations where the PNS may be accessed, including those at which many local and regional anesthesia techniques are routinely performed, such as by injection of pharmacological agents into a nerve or ganglion and surrounding areas, optionally with ultrasound guidance.

Injections may be performed, for example, with 17- to 22-gauge needles having inner diameters large enough to accommodate the herein described biocompatible capsules (which may, by way of non-limiting example, be 400-800 micrometers in diameter). Whereas introduction of capsules to the CNS may desirably employ a specialized catheter that is capable of injection into the brain without damaging CNS blood vessels, for peripheral sites as may be accessed for delivery to a PNS injection site, incidental vascular damage such as damage to blood vessels in mesenchymal tissue in the vicinity of an injection site may be of less particular concern.

Administration to a PNS injection site will preferably employ a needle that is able to penetrate through the skin and/or other adjacent tissue to the PNS site. See, e.g., Hadzic's Peripheral Nerve Blocks and Anatomy for Ultrasound-Guided Regional Anesthesia (New York School or Regional Anesthesia) (2011). Edited by Admir Hadzic, 722 pp. McGraw-Hill, New York, N.Y., ISBN-13:978-0-0715-4961-5. See also, e.g., Textbook of Regional Anesthesia and Acute Pain Management (2007) Edited by Admir Hadzic, 1259 pp., McGraw-Hill Education, New York, N.Y., ISBN 007144906X, 9780071449096. See also, e.g., Carneiro H M, Teixeira K I, de Ávila M P, Limongi R M, Magacho L. (2016) Comparison of Needle Path, Anesthetic Dispersion, and Quality of Anesthesia in Retrobulbar and Peribulbar Blocks. Reg Anesth Pain Med. 41(1):37-42. See also, e.g., Jeganathan V S1, Jeganathan V P. (2009) Sub-Tenon's anaesthesia: a well tolerated and effective procedure for ophthalmic surgery. Curr Opin Ophthalmol. 20(3):205-9.

In preferred embodiments, the step of administering comprises administering a therapeutically effective amount of the herein described biocompatible, semi-permeable alginate capsules containing CP cells, which in certain embodiments may comprise administering one or more capsules that each contain at least about 200, 400, 600, 800, 1000, 2000, 3000, 4000, 5000, 7500 or 9000 and not more than about 10,000 CP cells. In certain embodiments, no more than 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 capsules may be administered to the CNS (or PNS) injection site. In certain embodiments, capsules may be administered to 1, 2, 3, 4, 5, 6, 7, 8, 9, 10, 11, 12, or more CNS (or PNS) injection sites.

In certain embodiments administering the capsules to one or more CNS (or PNS) injection sites may comprise delivering a suspension comprising the capsules in a carrier solution, which may, for example, comprise at least one of

NaCl, artificial cerebrospinal fluid (CSF), ascorbate, or an anti-inflammatory agent. Exemplary anti-inflammatory agents may be selected from a non-steroidal anti-inflammatory drug (NSAID) or a steroid anti-inflammatory drug as known in the art (e.g., Brunton et al., (Eds.), Goodman & Gilman's The Pharmacological Basis of Therapeutics-12^{th} Ed. 2011 McGraw-Hill, New York), and/or may also include a connexin antagonist (e.g., Chen et al. 2014 Brain 137(Pt 8):2193; Zhang et al. 2014 FEBS Lett, 588(8):1365; Davidson et al. 2014 PLoS One 9(5):e96588).

Preferably at least 1, 5, 10, 20, 30, 40 or 50 percent of the encapsulated CP cells remain viable for at least six months after the step of administering. More preferably, at least 1, 5, 10, 20, 30, 40 or 50 percent of the encapsulated CP cells remain viable for at least 7, 8, 9, 10, 11, 12, 13, 14, 15, 16, 17, or 18 months after the step of administering. More preferably, at least 5, 10, 20, 30, 40 or 50 percent of the encapsulated CP cells remain viable for at least 7, 8, 9, 10, 11, 12, 13, 14, 15, 16, 17, 18, 19, 20, 21, 22, 23, or 24 months after the step of administering. More preferably, at least 1, 5, 10, 20, 30, 40 or 50 percent of the encapsulated CP cells remain viable for at least 2, 3, 4, 5, 6, 7, 8, 9, 10 or more years after the step of administering. Preferably exterior surfaces of the biocompatible capsules remain substantially free of extracellular matrix (ECM) deposition for at least six months after the step of administering, more preferably for at least 7, 8, 9, 10, 11, 12, 13, 14, 15, 16, 17, 18, 19, 20, 21, 22, 23, or 24 months after the step of administering, and more preferably for at least 2, 3, 4, 5, 6, 7, 8, 9, 10 or more years after the step of administering.

Monitoring the viability and status of implanted encapsulated CP cells may be achieved directly or indirectly by any of a variety of existing techniques. For instance, clinical assessment of the subject's neurological function, or positron emission tomography (PET) assessment of dopaminergic nerve function by neuronal ¹⁸F-fluorodopa and/or ¹¹C-tetrabenzine metabolism, biochemical analysis of CSF, or post-mortem analysis, may be indirectly indicative of restored functionality deriving from increased CSF production by the induced CP implants. As another example, CNS inflammation in vivo may be assessed by magnetic resonance imaging (MRI) techniques (Sibson et al., 2011 Meths. Mol. Biol. 711:379; McAteer et al., 2011 Meths. Mol. Biol. 680:103), or by methods that determine the levels in CSF and/or in the circulation of one or more biomarkers, such as C-reactive protein (CRP), monocyte chemotactic protein-1 (MCP-1), IL-6, or other markers (e.g., Lindqvist et al., 2013 Brain Behav. Immun. 33:183; Frodl et al., 2014 Prog Neuropsychopharmacol Biol Psychiatry, 48:295-303; Polachini et al., 2014 Neurosci. 266:266; Satizabal et al., 2012 Neurol. 78:720). These and related techniques may be employed to determine whether implanted encapsulated CP cells might be provoking an inflammatory reaction, after the procedure-related inflammation disappears, which would be expected to be accompanied by a foreign body response accompanied by ECM deposition.

In view of the surprising longevity of encapsulated CP cells following CNS implantation as described herein, certain further embodiments contemplate contacting such CP cells with a CP inducing agent as provided herein, simultaneously with or subsequent to the step of administering the encapsulated cells to a CNS injection site. In certain such embodiments, an Ommaya reservoir (Ommaya, 1963 Lancet 2:98; Dudrick, 2006 J. Parenter. Enteral. Nutr. 30 (1 Suppl):S47) may be implanted subcutaneously under the scalp of the subject to provide fluid communication from the reservoir to a catheter situated at or near the CNS site of capsule implantation. Via such a reservoir, or via other similar device by which fluid delivery to the vicinity of the CNS site may be achieved, the encapsulated CP tissue cells may be contacted with the CP inducing agent one or a plurality of times and at any time intervals (e.g., daily, 2, 3, 4, 5, or 6 times per week, weekly, biweekly, monthly, bimonthly, quarterly, semi-annually, annually, or any other interval over a period of 1, 2, 3, 4, 5, 6, 7, 8, 9, 10, 11, 12, 13, 14, 15 or more years) that may be identified for the subject beneficially to receive the CP inducing agent.

It will be appreciated that the practice of the several embodiments of the present invention will employ, unless indicated specifically to the contrary, conventional methods in virology, immunology, microbiology, molecular biology and recombinant DNA techniques that are within the skill of the art, and many of which are described below for the purpose of illustration. Such techniques are explained fully in the literature. See, e.g., Current Protocols in Molecular Biology or Current Protocols in Immunology, John Wiley & Sons, New York, N.Y.(2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

As used in this specification and the appended claims, the singular forms “a,” “an” and “the” include plural references unless the content clearly dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Each embodiment in this specification is to be applied mutatis mutandis to every other embodiment unless expressly stated otherwise.

EQUIVALENTS: While particular steps, elements, embodiments and applications of the present invention have been shown and described herein for purposes of illustration, it will be understood, of course, that the invention is not limited thereto since modifications may be made by persons skilled in the art, particularly in light of the foregoing teachings, without deviating from the spirit and scope of the invention. Accordingly, the invention is not limited except as by the appended claims.

The following Examples are presented by way of illustration and not limitation.

### EXAMPLES

### Example 1

### Selection of Choroid Plexus (CP) Cell-Containing Capsules for Elevated Cerebrospinal Fluid (CSF) Production

This example describes selection of CP cell-containing capsules for elevated CSF production using the CSF component VEGF as a representative indicator of CSF production.

Neonatal porcine choroid plexus tissue was processed and encapsulated in alginate capsules essentially as described in US2009/0047325 and US2009/0214660. Briefly, CP tissue was sterilely dissected from neonatal pig brains, finely chopped with scissors, digested with collagenase and thermolysin, and passed through a 550 μm stainless steel filter, pelleted and gently resuspended to obtain tissue fragments comprising cell clusters of about 50-200 μm in diameter. CP cell clusters were separated from blood cells by unit gravity sedimentation twice for 40 minutes at room temperature. The settled CP cells were resuspended in RPMI medium/2% neonatal porcine serum at a density of approximately 3,000 clusters per mL and cultured in ultra low attachment flasks for 6-7 days as described, yielding spherical, ovoid and branched CP cell clusters.

The cell clusters were incubated in sterile saline solution containing high mannuronic acid containing alginate, droplet-sprayed through a droplet generator into a 109 mM calcium chloride gelation solution, and successively coated with poly-L-ornithine and alginate, to obtain semi-permeable capsules substantially all of which were about 400 μm to about 800 μm in diameter. The capsules were then treated with 55 mM iso-osmolar sodium citrate for 2 minutes in a rolling 50 mL tube. Capsules were maintained in culture medium containing serum and aliquots sampled to confirm cell viability.

The amounts of VEGF secreted per cell were compared in aliquots of equivalent numbers of unselected (random) CP cell-containing capsules and selected CP cell-containing capsules. Selected CP cell-containing capsules were hand-picked, on the basis of direct microscopic observation, for the presence of 200-to-10,000 encapsulated cells per capsule, where the capsules exhibited a smooth exterior surface uninterrupted by protruding cells or cellular processes from the capsule interior or by superficially attached cells or tissue fragments. Culture wells of a 24-well multi-well plate were seeded either with 500 unselected (random) CP cell-containing capsules or 500 selected capsules and cultured at 37° C. for 24 hours. Aliquots of supernatant fluids were collected and assayed for VEGF using an ELISA kit (Human VEGF Quantikine™ ELISA, Cat. #DVE00, R & D Systems, Minneapolis, Minn.) according to the manufacturer's instructions. Aliquots of the cell cultures were also collected for DNA quantification using a Quant iT™ Picogreen dsDNA assay according to the supplier's instructions (Cat. #P7589, Life Technologies, Inc./Thermo Fisher Scientific, Grand Island, N.Y.)?] to determine the relative number of cells in each well.

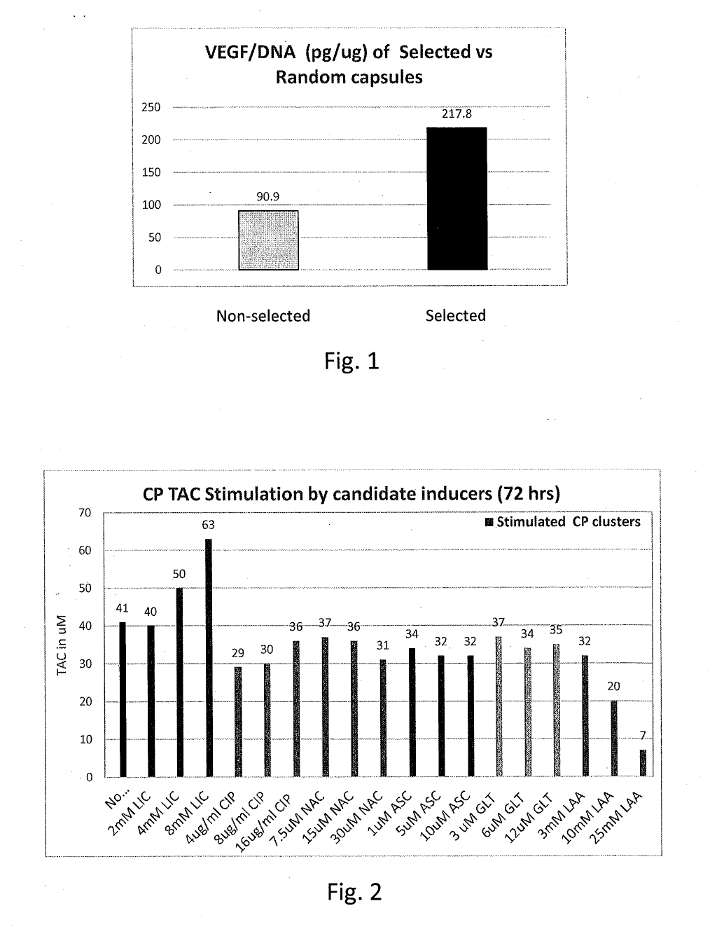

Comparative DNA quantification of the culture wells revealed that wells receiving 500 selected capsules (200-10,000 encapsulated cells per capsule) contained three times as much DNA as wells that had received 500 unselected (random) capsules. Surprisingly, supernatant fluids of wells that had received 500 selected capsules contained six times as much VEGF than the supernatants from unselected (random) capsule cultures. When the samples were normalized to DNA content as a reflection of the amount of VEGF secreted on a per cell basis, selected capsules surprisingly were found to secrete slightly more than twice as much VEGF per μg of DNA than unselected capsules (FIG. 1). This result was unexpected insofar as a correspondence between increased VEGF production per cell and higher cell density has not previously been reported.

### Example 2

### Identification of a Choroid Plexus Inducing Agent

This example describes the identification of an agent that induces mammalian choroid plexus (CP) cells to produce a CSF component at a level that is greater than the level at which CP cells produce the CSF component in the absence of the inducing agent. CSF is known to contain multiple components that function as antioxidants (e.g., Kolmakova et al., 2010 Neurochem. J. 4:41); collectively the antioxidant properties of these components may be referred to as the total antioxidant capacity (TAC).

Choroid plexus (CP) cell clusters comprising CP cells (5×10³ clusters/mL) were prepared as described above in Example 1 but without encapsulation and cultured at 37° C. in a 5% CO₂ incubator for 24 or 72 hours in vitro in 24-well ultra-low (cell) attachment plates, and culture supernatants were tested for total antioxidant capacity (TAC) using the OxiSelect™ TAC assay (Cat. No. STA-360, Cell Biolabs, Inc., San Diego, Calif.) according to the manufacturer's instructions. To avoid interference in the TAC assay from medium components, cultures were incubated in serum-free, phenol-free RPMI media supplemented with 10 mM nicotinamide. A panel of candidate CP inducing agents was also tested for effects on TAC elaboration by CP cells. Control wells that received the culture medium alone or with each candidate CP inducing agent, but no CP cell clusters, were also incubated and tested for TAC. Representative candidate CP inducing agents were as shown in Table 1:

TABLE 1
CANDIDATE CP INDUCING AGENTS

| Candidate | | | | | | |
|---|---|---|---|---|---|---|
| Inducer | Concentration 1 | | Concentration 2 | | Concentration 3 | |
| LiCl | 2 | mM | 4 | mM | 8 | mM |
| Ciproxin | 4 | ug/ml | 8 | ug/ml | 16 | ug/ml |
| Ascorbic acid | 1 | μM | 5 | μM | 10 | μM |
| Lactic acid | 3 | mM | 10 | mM | 25 | mM |
| N Acetyl | 7.5 | μM | 15 | μM | 30 | μM |
| Cysteine | | | | | | |
| Glutathione | 3 | μM | 6 | μM | 12 | μM |
| Nicotinamide | 10 | mM | 20 | mM | 40 | mM |

The TAC assay results are shown in FIG. 2. Among the candidate CP inducing agents that were tested, lithium chloride promoted the release by CP cells of elevated TAC levels that were readily detectable after 72 hours.

Therefore, in a further experiment, CP clusters (5×10³ clusters/mL) were incubated for 72 hours in serum-free, phenol-free RPMI medium containing 10 mM nicotinamide in the presence of varying LiCl concentrations, to determine whether CP cells release antioxidant activity into the supernatant in response to LiCl in a dose-dependent manner. The results are shown in FIG. 3A, in which detectable TAC levels released by CP clusters in response to LiCl are presented, after subtracting the background TAC level released by CP clusters in the absence of LiCl, and correcting for any TAC signal detected in the respective medium/LiCl without CP cells present.

To determine that certain inducers can increase the secretion of CSF components by CPs that have been encapsulated, 1000 CP-containing capsules were cultured in 24-well multi-well plate with or without various inducers including LiCl at 37° C. for 24 hours. Aliquots of supernatant fluids were collected and assayed for VEGF as described in EXAMPLE 1. Aliquots of the cell cultures were also collected for DNA quantification to determine the relative number of cells in each well as described in EXAMPLE 1. The results are shown in FIG. 3B, in which both LiCl as well as lithium carbonate induced VEGF secretion by encapsulated CP above the level secreted by encapsulated CPs cultured without inducers. In addition, two other agents, taurine and MitoQ, also induced VEGF secretion by encapsulated CPs.

In a further experiment to identify genes for which the expression levels were altered in CP cells following contact with an inducing agent as provided herein, CP clusters (5×10³ clusters/mL) containing CP cells, obtained from 12-13 day-old piglets and cultured in vitro for 20-22 days, were incubated for 72 hours in serum-free, phenol-free RPMI medium containing 10 mM nicotinamide in the absence (controls) or presence (inducing agent-treated) of 12 mM LiCl. Cell pellets were harvested and 0.25 ml of TRI™ Reagent (Catalog # T9424, Sigma-Aldrich Corporation, St. Louis, Mo.) was added to lyse the cells. The lysate was pipetted up and down approximately 10 times and stored at −80° C. until RNA-Seq analysis. The results from controls and treated CP clusters were compared and are shown in FIG. 8, which lists multiple porcine genes for which expression levels increased (FIG. 8A-E) or decreased (FIG. 8F-K) in CP cells following exposure to LiCl, and which genes were identified as genes that encode known CSF components. Corresponding human genes encoding human CSF components were identified by established orthology analysis (Groenen et al., 2012 Nature 491(7424):393-8).

### Example 3

### Long-Term In Vivo Survival of CNS-Implanted Encapsulated Xenogeneic Choroid Plexus (CP) Cells Without Immunosuppressive Regimen

Alginate-encapsulated neonatal porcine choroid plexus (CP) clusters comprising 200 to 10,000 CP cells per capsule were prepared as described above and in US2009/0047325 and US2009/0214660. Capsules (10 per recipient) were surgically implanted into the striatum of multiple anesthetized Sprague-Dawley rats using a catheter designed for rodent brain implantation. Animals were maintained for 1-16 months (for initial experiments, monthly time points were collected starting at one month; subsequent experiments provided confirmatory data starting at 12 months) and at each monthly interval sample animals were humanely sacrificed for histological examination of post-mortem brains. No anti-inflammatory or immunosuppressive treatments were administered.

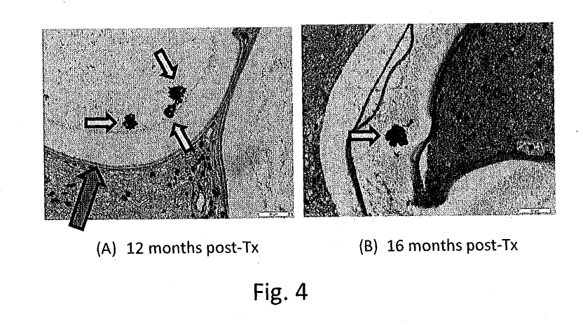

Histological findings indicated that at each time point for sacrifice (9, 12 or 16 months), living cells were present within the implanted capsules, and there was little or no detectable evidence of a host immunological rejection, inflammatory reaction or foreign body response (e.g., fibrotic scarring) to the capsules. See FIG. 4. Post-mortem brain tissue was processed for immunohistological staining of pigment epithelium derived factor (PEDF, also known as SerpinF1), a multifunctional CSF component produced by CP cells (Fernandez-Garcia et al., 2007 J. Mol. Med. (Berl.) 85:15-22; Barnstable et al., 2004 Prog. Retin. Eye Res. 23:561; Becerra et al., 1995 J. Biol. Chem. 270:25992; Orgaz et al., 2011 Melanoma Res. 21:285). PEDF was readily detectable in CP cells present in clusters within the clusters (FIG. 4) as well as in surrounding rat brain tissues.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating a subject known to have or suspected of having a nervous system disease, comprising:
(a) selecting one or more semi-permeable biocompatible capsules in which are encapsulated choroid plexus (CP) tissue fragments that are obtained by either or both of mechanical and enzymatic dissociation of mammalian choroid plexus tissue to obtain CP cell clusters that are about 50 μm to about 200 μm in diameter and that comprise CP epithelial cells, substantially all of said capsules being about 400 μm to about 800 μm in diameter and having about 200 to about 10,000 CP cells per capsule;
(b) administering one or a plurality of said capsules to a central nervous system (CNS) injection site or to 2, 3, 4, 5, 6, 7, 8, 9, 10, 11, or 12 CNS injection sites in the subject; and
(c) prior to, simultaneously with, or subsequent to said step (b) of administering, contacting the choroid plexus tissue cells in the one or a plurality of capsules with a choroid plexus inducing agent that induces the choroid plexus tissue cells to produce one or more cerebrospinal fluid (CSF) components at a level that is altered relative to the level at which the choroid plexus tissue cells produce said one or more cerebrospinal fluid (CSF) components without said step of contacting.

2. A method of treating a subject known to have or suspected of having a nervous system disease, comprising:
(a) selecting one or more semi-permeable biocompatible capsules in which are encapsulated in vitro differentiated choroid plexus (CP) cells that are obtained by culturing a population of pluripotent cells under conditions and for a time sufficient to obtain a plurality of in vitro differentiated choroid plexus (CP) cells, substantially all of said capsules being about 400 μm to about 800 μm in diameter and having about 200 to about 10,000 CP cells per capsule;
(b) administering one or a plurality of said capsules to a central nervous system (CNS) injection site or to 2, 3, 4, 5, 6, 7, 8, 9, 10, 11, or 12 CNS injection sites in the subject; and
(c) prior to, simultaneously with, or subsequent to said step (b) of administering, contacting the in vitro differentiated choroid plexus (CP) cells in the one or a plurality of capsules with a choroid plexus inducing agent that induces the in vitro differentiated choroid plexus (CP) cells to release one or more cerebrospinal fluid (CSF) components at a level that is altered relative to the level at which the choroid plexus tissue cells produce said one or more cerebrospinal fluid (CSF) components prior to said step of contacting.

3. The method of claim 1 wherein the choroid plexus inducing agent induces production of one or more CSF components at a level that is greater than the level at which the choroid plexus tissue cells produce said one or more cerebrospinal fluid (CSF) components without said step of contacting.

4. The method of claim 1 wherein the step of contacting the CP cells with the choroid plexus inducing agent takes place prior to said step (b) of administering.

5. The method of claim 1 wherein the choroid plexus inducing agent comprises one or more agents selected from:
(a) a Wnt signaling pathway agonist,
(b) a GSK3β inhibitor,
(c) a beta-catenin activator,
(d) an antioxidant, and
(e) 1,25-dihydroxyvitamin D₃.

6. The method of claim 5 wherein:
(1) the Wnt signaling pathway agonist is selected from WAY-316606 (SFRP inhibitor), IQ1 (PP2A activator), QS11 (ARFGAP1 activator), (hetero)arylpyrimidine, or 2-amino-4-[3,4-(methylenedioxy) benzyl-amino]-6-(3-methoxyphenyl) pyrimidine, Norrin, R-spondin-1, R-spondin-2, R-spondin-3, R-spondin-4,
(2) the GSK3β inhibitor is selected from SB-216763, BIO (6-bromoindirubin-3′-oxime), lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or another lithium salt that comprises cationic lithium,
(3) the beta-catenin activator is selected from deoxycholic acid (DCA) and a compound of FIGS. 5, and
(4) the antioxidant is selected from a 10-(6′-ubiquinoyl) decyltriphenylphosphonium salt (mitoquinol, MITOQ®), ubiquinol (coenzyme Q), tocopherols, tocotrienol (vitamin E), α-tocopherol, γ-tocopherol, 2-aminoethanesulfonic acid (taurine), ascorbic acid, glutathione, and melatonin.

7. The method of claim 1 wherein the mammalian choroid plexus tissue is selected from:
(a) choroid plexus tissue from a mammal that is xenogeneic or allogeneic relative to the subject,
(b) choroid plexus tissue that comprises porcine, ovine, bovine, caprine, or non-human primate choroid plexus tissue, and
(c) porcine choroid plexus tissue that comprises fetal or neonatal choroid plexus tissue.

8.-9. (canceled)

10. The method of claim 7 wherein at least one of:
(a) the porcine fetal or neonatal choroid plexus tissue is substantially free of human pathogens,
(b) the porcine fetal or neonatal choroid plexus tissue is substantially free of human-tropic transmissible porcine endogenous retroviruses,
(c) at least one of: (i) the porcine fetal or neonatal choroid plexus tissue is substantially incapable of producing infectious human-tropic porcine endogenous retroviruses (PERVs), or (ii) the fetal or neonatal choroid plexus tissue is obtained from an animal that lacks PERV genes,
(d) the porcine fetal or neonatal choroid plexus tissue is obtained from an animal that lacks a PERV-C env gene which is capable of recombination with a PERV-A env gene.

11.-16. (canceled)

17. The method of claim 1 wherein either or both of:
the capsules do not elicit chronic inflammation at the CNS injection site, and
(ii) administration of an immunosuppressant agent to the subject is not required to ameliorate immunological rejection of the capsules at the CNS injection site.

18. The method of claim 1 wherein the one or more CSF components comprise at least one of (i) one or more growth factors, (ii) one or more CSF antioxidants, (iii) one or more chemotactic factors, (iv) one or more chaperone proteins, or (v) one or more CP products as presented in FIG. 7A-J.

19. The method of claim 18 wherein:
(a) the one or more growth factors are selected from IGF-1, IGF-II, FGF-1, bFGF (FGF-2), FGF-9, FGF-12, FGF-18, TGF-β1, TGF-β2, TGF- β3, VEGF, VEGF-2, VEGF-B, VEGF-C, EGF, growth hormone (GH), BMP-1, BMP-2, BMP-4, BMP7, BMP-11, BMP-15, GDF-1, GDF-7, GDF-8, GDF-9, nerve growth factor (NGF), PEDF (pigment epithelium derived factor, also known as SerpinF1), glucagon-like peptide-1 (GLP-1), IGF2, BDNF, NT-3, NT-4, GDF-15, GDNF, connective tissue growth factor (CTGF), axotrophin, heparin-binding EGF-like growth factor (HB-EGF), platelet derived growth factor-alpha (PDGF-α), Keratinocyte growth factor (KGF), or neurite growth-promoting factor-2/midkine (NEGF2);
(b) the one or more CSF antioxidants are selected from ceruloplasmin, superoxide dismutase-1 (SOD-1), superoxide dismutase-2 (SOD-2, Mn-type), superoxide dismutase copper chaperone (CCS), DJ-1/PARK7, catalase, selenoproteins (I, M, N, P, S, T, W, X, 15 kDa) , glutathione S-transferase, glutathione reductase, glutathione peroxidase, hydroxyacyl glutathione hydrolase or thioredoxin;
(c) the one or more chemotactic factors are selected from alveolar macrophage-derived chemotactic factor-I (AMCF-I), AMCF-II, stromal cell-derived factor-2, chemokine (CXC motif) ligand 2, chemokines (CCL8, CCL16, CCL19, CCL21, CCL25, CXCL2, CXCL4, CXCL9, CXCL12, CXCL13, CXCL14), chemokine (CXC motif) receptor-4, a chemokine-like factor super family (CKLF-3, -6, -7), or neurite growth-promoting factor-2/midkine (NEGF2); or
(d) the chaperone proteins are selected from transthyretin, lipocalin-type prostaglandin D synthase/β-trace (L-PGDS), apolipoproteins (A, B, C, D, E, H, J, M, N, R), lipocalin-6, lipocalin-7, cystatin B, cystatin C, cystatin EM, cystatin 11, a heat shock protein (HSP) family member, or DJ-1/PARK7.

20. The method of claim 1 wherein within each capsule the CP cells are present in a core volume of less than one microliter.

21. The method of claim 1 wherein the step of administering comprises administering one or more capsules that each contain at least about 200, 400, 600, 800, 1000, 2000, 3000, 4000, 5000, 7500 or 9000 and not more than about 10,000 CP cells.

22. The method of claim 21 wherein the one or more capsules each contain at least about 400, 600, 800, 1000, 1500, 2000, 2500, 3000, 3500, 4000, 4500, 5000, 5500, 6000, 6500, 7000, or 7500 and not more than about 8000 cells.

23. The method of claim 1 wherein the step of administering comprises administering a therapeutically effective amount of the capsules to the CNS injection site.

24. The method of claim 23 which comprises administering no more than 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450,500, 550, 600, 650, 700, 750, 800, 850, 900, 950, 1000, 1100, 1200, 1300, 1400, 1500, 1600, 1700, 1800, 1900, or 2000 capsules to the 1, 2, 3, 4, 5, 6, 7, 8, 9, 10, 11, or 12 CNS injection sites.

25. The method of claim 1 wherein at least 1, 5, 10, 20, 30, 40 or 50 percent of the encapsulated CP cells remain viable for at least six months after the step of administering.

26. The method of claim 1 wherein exterior surfaces of the biocompatible capsules are substantially free of extracellular matrix deposition for at least one year after the step of administering.

27. The method of claim 1 wherein administering the capsules to the CNS injection site comprises delivering a suspension comprising the capsules in a carrier solution.

28. The method of claim 27 wherein the carrier solution comprises at least one of NaCl, artificial cerebrospinal fluid (CSF), ascorbate, or an anti-inflammatory agent.

29. The method of claim 28 wherein the anti-inflammatory agent is selected from a non-steroidal anti-inflammatory drug (NSAID), a steroid anti-inflammatory drug, and a connexin antagonist.

30. The method of claim 1 wherein the subject is a human or a non-human mammal.

31. The method of claim 1 wherein the subject is known to have a nervous system disease.

32. The method of claim 31 wherein the nervous system disease is selected from (a) a neurodegenerative disease that is characterized by death of neurons, and (b) a nervous system disease that is selected from Parkinson's disease, Alzheimer's disease, Huntington's disease, amyotrophic lateral sclerosis (ALS, also known as motor neurone disease), ataxia-telangiectasia, progressive bulbar palsy, progressive muscular atrophy, dementia with Lewy bodies, multiple system atrophy, spinocerebellar ataxia type 1 (SCA 1), or an age-related neurodegenerative disorder.

33. The method of claim 31 wherein the nervous system disease is selected from
(a) a nervous system disease disease that is characterized by a decrease in a level of at least one nerve cell function, relative to the level of said nerve cell function in a control subject known to be free of the nervous system disease,
(b) the nervous system disease disease of (a) that is selected from Parkinson's disease, Alzheimer's disease, Huntington's disease, amyotrophic lateral sclerosis, and depression,
(c) a nervous system disease that is characterized by an increase in a level of at least one nerve cell function, relative to the level of said nerve cell function in a control subject known to be free of the nervous system disease,
(d) the nervous system disease of (c) that is selected from psychosis, schizophrenia, epileptic seizures, ischemic stroke, and insomnia associated with restless leg syndrome,
(e) a nervous system disease that is characterized by presence in the subject of cerebrospinal fluid (CSF) that comprises an altered level of one or more cerebrospinal fluid (CSF) components, relative to the level of said CSF component or components in a control subject known to be free of the nervous system disease,
(f) the nervous system disease of (e) that is selected from Alzheimer's disease and diabetes mellitus,
(g) a nervous system disease that is characterized by presence in the subject of an altered level of at least one choroid plexus function, relative to the level of said choroid plexus function in a control subject known to be free of the nervous system disease,
(h) the nervous system disease of (g) that is selected from Sturge-Weber syndrome and Klippel-Trenaunay-Weber syndrome,
(i) a nervous system disease that is characterized by an increase in a level of abnormally folded protein deposits in brain tissue of the subject, relative to the level of abnormally folded protein deposits in a control subject known to be free of the nervous system disease, and
(j) the disease of (i) that is selected from cerebral amyloid angiopathy, hereditary cerebral hemorrhage with amyloidosis-Icelandic type (HCHWA-I), cerebral hemorrhage with amyloidosis-Dutch type (HCHWA-D), meningocerebrovascular and oculoleptomeningeal amyloidosis, gelsolin-related spinal and cerebral amyloid angiopathy, familial amyloidosis-Finnish type (FAF), vascular variant prion cerebral amyloidosis, familial British dementia (FBD) (also known as familial cerebral amyloid angiopathy-British type or cerebrovascular amyloidosis-British type), familial Danish dementia (also known as heredopathia ophthalmo-oto-encephalica), familial transthyretin (TTR) amyloidosis, and PrP cerebral amyloid angiopathy (PrP-CAA).

34-36. (canceled)

37. The method of claim 1 wherein the nervous system disease is a central nervous system (CNS) disease.

38. The method of claim 37 wherein the CNS disease is at least one of (i) a neurodegenerative disease that is characterized by death of CNS neurons, and (ii) a CNS disease characterized by a decrease in a level of at least one CNS nerve cell function, relative to the level of said CNS nerve cell function in a control subject known to be free of the CNS disease, and iii) a CNS disease characterized by an increase in a level of at least one CNS nerve cell function, relative to the level of said CNS nerve cell function in a control subject known to be free of the CNS disease, wherein said CNS neurons and CNS nerve cell are present in at least one of brain, spinal cord, retina, optic nerve, cranial nerve, olfactory nerve or olfactory epithelium.

39. The method of claim 1 wherein the nervous system disease is a peripheral nervous system (PNS) disease.

40. The method of claim 39 wherein the PNS disease is at least one of (i) a neurodegenerative disease that is characterized by death of PNS neurons, and (ii) a PNS disease characterized by a decrease in a level of at least one PNS nerve cell function, relative to the level of said PNS nerve cell function in a control subject known to be free of the PNS disease, and iii) a PNS disease characterized by an increase in a level of at least one PNS nerve cell function, relative to the level of said PNS nerve cell function in a control subject known to be free of the PNS disease, wherein said PNS neurons and PNS nerve cell are present in at least one of a peripheral ganglion or a peripheral nerve.

41. The method of claim 1 wherein the CNS injection site is in brain tissue of the subject.

42. The method of claim 1 wherein the CNS injection site is in a brain ventricle of the subject.

43. The method of claim 1 wherein the CNS injection site in the subject is selected from:
(a) a CNS site that comprises a target site for nerve cell fibers that are affected by the nervous system disease,
(b) a CNS site that contains neuronal cells that are at risk of dying due to the nervous system disease,
(c) a CNS site that contains neuronal cells that are at risk of a decrease in a level of at least one nerve cell function, relative to the level of said nerve cell function in a control subject known to be free of the nervous system disease,
(d) a CNS site that contains neuronal cells that are at risk of an increase in a level of at least one nerve cell function, relative to the level of said nerve cell function in a control subject known to be free of the nervous system disease,
(e) a CNS site that is selected so that the capsules are substantially free of contact with blood, and
(f) a CNS site that is selected so that CSF components secreted by the capsules subsequent to the step of administering are distributed by CSF circulation throughout the subject's brain.

44. The method of claim 1, wherein the biocompatible capsule comprises a core layer of a high mannuronic acid alginate cross-linked with a cationic cross-linking agent, an intermediate layer of polycations forming a semi-permeable membrane, and an outer layer of a high mannuronic acid alginate cross-linked with a cationic cross-linking agent, wherein the high mannuronic acid alginate in the core and outer layers is the same or different and contains between from about 50% to about 95% mannuronic acid residues, wherein the polycation layer is not comprised of poly-L-lysine.

45. The method of claim 44 wherein the high mannuronic acid alginate has an average molecular weight of greater than about 300 kDa and not more than 1000 kDa and the polycation layer is formed from a polycationic agent having an average molecular weight of between 10 and 40 kDa.

46. The method of claim 1 wherein administering the capsules to the CNS site comprises delivering the capsules through a catheter.

47. The method of claim 46 wherein delivering comprises controllably positioning the catheter with a stereotactic apparatus.

48. The method of claim 47 wherein the stereotactic apparatus comprises a stereotactic apparatus or a modified stereotactic apparatus that is selected from a deep brain stimulator (DBS) microdriver, a frameless stereotactic head frame, a skull-mounted aiming device, a Leksell frame, and a Cosman-Roberts-Wells frame.

49. The method of claim 46 wherein the catheter comprises an external catheter, an obdurator, a plunger, and a delivery catheter.

50. The method of claim 31 wherein the nervous system disease is selected from Parkinson's disease, Alzheimer's disease, Huntington's disease, amyotrophic lateral sclerosis (ALS), prion disease, motor neuron disease, spinocerebellar ataxia, spinal muscular atrophy, multiple system atrophy-Parkinson type, multiple system atrophy-cerebellar type, essential tremor, progressive supronuclear palsy, dyskinesias, dementia with Lewy bodies, essential tremor, drug-induced Parkinsonism, ataxia-telangiectasia, spinocerebellar ataxia, cerebellar degeneration, cerebral atrophy, olivopotocerebellar atrophy, corticobasal degeneration, dyssynergia cerebellaris myoclonica, Friedreich's ataxia; a static nervous diseases, stroke, central pain syndrome, chronic pain, migraine, glossopharyngeal neuralgia, a seizure disorder, epilepsy, cerebral palsy; a trauma-related CNS diseases, Gerstmann's syndrome, locked-in syndrome, spinal cord injury, a progressive neurodegenerative diseases, progressive neurodegenerative disease associated with aging and dementia, Alzheimers disease, Parkinson's disease, frontotemporal dementia, Gerstmann-Straussler-Scheinker disease, giant axonal neuropathy, hereditary neuropathies, infantile neuroaxonal dystrophy, Krabbe disease, Landau-Kleffner syndrome, Tabes dorsalis, a disease of motor neurons and neuromuscular junctions, spinal muscular atrophy, Kennedy's disease, monomelic amyotrophy, dystonias, hereditary spastic paraplegia, Isaacs' syndrome, Lambert-Eaton myasthenic syndrome, motoneuron diseases, restless legs syndrome, Tourette syndrome; inflammatory diseases of the CNS, multiple sclerosis; drug or toxin-induced CNS diseases, neuroleptic malignant syndrome, tardive dyskinesia, Wilson disease, neurotoxicity; nervous system disease of metabolic failure, Refsum disease, a nervous system infectious disease, meningitis, acute disseminated encephalomyelitis, Guillain-Barre syndrome, neurological complications of AIDS, botulism, tetanus, neurosyphilis, poliomyelitis, rabies, HIV/AIDS, prion diseases, Naegleria fowleri (amoebic brain infection); neurocysticerosis; a neuropsychiatric disease, depression, mood disorders; obsessive-compulsive disorder, eating disorder, addiction, anxiety-related disorder, bipolar disorder, attention-deficit-hyperactivity disorder, autism, schizophrenia; a neuroendocrine disease, narcolepsy, insomnia, a diseases associated with or characterized by one or more of neuronal death, glutamate toxicity, protein aggregates or deposits, or amyloid plaque formation, cerebral amyloid angiopathy, hereditary cerebral hemorrhage with amyloidosis-Icelandic type (HCHWA-I), cerebral hemorrhage with amyloidosis-Dutch type (HCHWA-D), meningocerebrovascular and oculoleptomeningeal amyloidosis, gelsolin-related spinal and cerebral amyloid angiopathy, familial amyloidosis-Finnish type (FAF), vascular variant prion cerebral amyloidosis, familial British dementia (FBD) (also known as familial cerebral amyloid angiopathy-British type or cerebrovascular amyloidosis-British type), familial Danish dementia (also known as heredopathia ophthalmo-oto-encephalica), familial transthyretin (TTR) amyloidosis, PrP cerebral amyloid angiopathy (PrP-CAA); a nervous system disease of mitochondrial dysfunction, a nervous system disease of mitochondrial dysfunction that comprises reactive oxygen species (ROS) production levels in excess of ROS production levels found in normal, healthy control subjects; a brain derived neurotrophic factor-related disorders, bipolar disorders, Rett Syndrome, and Rubinstein-Taybi Syndrome.

51. A method of treating a subject known to have or suspected of having a nervous system disease, comprising:
(a) selecting one or more semi-permeable biocompatible capsules in which are encapsulated choroid plexus (CP) tissue fragments that are obtained by either or both of mechanical and enzymatic dissociation of mammalian choroid plexus tissue to obtain CP cell clusters that are about 50 μm to about 200 μm in diameter and that comprise CP epithelial cells, substantially all of said capsules being about 400 μm to about 800 μm in diameter and having about 200 to about 10,000 CP cells per capsule;
(b) administering one or a plurality of said capsules to a peripheral nervous system (PNS) injection site or to 2, 3, 4, 5, 6, 7, 8, 9, 10, 11, or 12 PNS injection sites in the subject; and
(c) prior to, simultaneously with, or subsequent to said step (b) of administering, contacting the choroid plexus tissue cells in the one or a plurality of capsules with a choroid plexus inducing agent that induces the choroid plexus tissue cells to produce one or more cerebrospinal fluid (CSF) components at a level that is altered relative to the level at which the choroid plexus tissue cells produce said one or more cerebrospinal fluid (CSF) components without said step of contacting.

52. A method of treating a subject known to have or suspected of having a nervous system disease, comprising:
(a) selecting one or more semi-permeable biocompatible capsules in which are encapsulated in vitro differentiated choroid plexus (CP) cells that are obtained by culturing a population of pluripotent cells under conditions and for a time sufficient to obtain a plurality of in vitro differentiated choroid plexus (CP) cells, substantially all of said capsules being about 400 μm to about 800 μm in diameter and having about 200 to about 10,000 CP cells per capsule;
(b) administering one or a plurality of said capsules to a peripheral nervous system (PNS) injection site or to 2, 3, 4, 5, 6, 7, 8, 9, 10, 11, or 12 PNS injection sites in the subject; and
(c) prior to, simultaneously with, or subsequent to said step (b) of administering, contacting the in vitro differentiated choroid plexus (CP) cells in the one or a plurality of capsules with a choroid plexus inducing agent that induces the in vitro differentiated choroid plexus (CP) cells to release one or more cerebrospinal fluid (CSF) components at a level that is altered relative to the level at which the choroid plexus tissue cells produce said one or more cerebrospinal fluid (CSF) components prior to said step of contacting.

53. The method of claim 51 wherein the choroid plexus inducing agent induces production of one or more CSF components at a level that is greater than the level at which the choroid plexus tissue cells produce said one or more cerebrospinal fluid (CSF) components without said step of contacting.
